(19) United States
(12) Patent Application Publication
(10) Pub. No.: US 2024/0400716 A1
(43) Pub. Date: Dec. 5, 2024

(54) MOLECULES THAT BIND TO PROSTATE SPECIFIC MEMBRANE ANTIGEN (PSMA) POLYPEPTIDES

(71) Applicant: University of Pittsburgh - Of the Commonwealth System of Higher Education, Pittsburgh, PA (US)

(72) Inventors: Dimiter Stanchev Dimitrov, Pittsburgh, PA (US); Dontcho V. Jelev, Pittsburgh, PA (US); Wei Li, Pittsburgh, PA (US); Zehua Sun, Pittsburgh, PA (US); John W. Mellors, Pittsburgh, PA (US)

(21) Appl. No.: 18/690,837

(22) PCT Filed: Nov. 8, 2022
(86) PCT No.: PCT/US22/49288

§ 371 Date: Mar. 11, 2024

Related U.S. Application Data
(60) Provisional application No. 63/277,481, filed on Nov. 9, 2021.

(51) Int. Cl. C07K 16/30; A61K 47/68

(52) U.S. Cl. CPC C07K 16/3069; A61K 47/6869; C07K 2317/622; C07K 2317/92

(57) ABSTRACT

This document provides methods and materials involved in binding a binder (e.g., an antibody, antigen binding fragment, antibody domain, CAR, cell engager, and/or ADC) to a PSMA polypeptide. For example, binders (e.g., antibodies, antigen binding fragments, antibody domains, CARs, cell engagers, and/or ADCs) that bind to a PSMA polypeptide and methods and materials for using one or more such binding molecules to treat a mammal (e.g., a human) having cancer are provided.

### CROSS-REFERENCE TO RELATED APPLICATIONS

This application claims the benefit of U.S. Provisional Application Ser. No. 63/277,481, filed Nov. 9, 2021. The disclosure of the prior application is considered part of (and is incorporated by reference in) the disclosure of this application.

### BACKGROUND

### 1. Technical Field

This document relates to methods and materials involved in binding a molecule (e.g., an antibody, a fragment of an antibody, an antibody domain, a chimeric antigen receptor (CAR), a cell engager, or an antibody-drug conjugate (ADC)) to a PSMA polypeptide. For example, this document provides binders (e.g., antibodies, antigen binding fragments, antibody domains, CARs, cell engagers, or ADCs) that bind to a PSMA polypeptide and methods and materials for using such binders to treat cancer. This document also provides cells (e.g., host cells) designed to express one or more binders (e.g., antibodies, antigen binding fragments, antibody domains, CARs, or cell engagers) having the ability to bind to a PSMA polypeptide and methods and materials for using such cells to treat cancer.

### 2. Background Information

PSMA is highly expressed in the epithelial cells of the prostate and is also detected in epithelial cells of other tissues. PSMA is integral membrane ectopeptidase normally expressed on the basal side of the cell membrane. In prostate cancer (PC) PSMA is transferred to the luminal side of the membrane and its expression level is increased up to 1000 times. Increased PSMA expression correlates with advanced disease stage and poor prognosis. PSMA is also expressed at high levels in nonprostatic tumor neovasculature.

### SUMMARY

This document provides methods and materials involved in binding a molecule (e.g., an antibody, an antigen binding fragment, an antibody domain, a CAR, a cell engager, or an ADC) to a PSMA polypeptide. For example, this document provides binders (e.g., antibodies, antigen binding fragments, antibody domains, CARs, cell engagers, or ADCs) that bind to a PSMA polypeptide and methods and materials for using one or more such binders to treat a mammal (e.g., a human) having cancer.

This document also provides cells (e.g., host cells) designed to express one or more binders (e.g., antibodies, antigen binding fragments, antibody domains, CARs, or cell engagers) having the ability to bind to a PSMA polypeptide and methods and materials for using such cells to treat cancer.

As described herein, binders (e.g., one or more antibodies, one or more antigen binding fragments, one or more antibody domains, one or more CARs, one or more cell engagers, and/or one or more ADCs) can be designed to have the ability to bind to a PSMA polypeptide. For example, a binder (e.g., an antibody, an antigen binding fragment, an antibody domain, a CAR, a cell engager, or an ADC) provided herein can have the ability to bind to a polypeptide comprising, consisting essentially of, or consisting of the amino acid sequence of a human PSMA polypeptide as set forth in SEQ ID NO:273 or SEQ ID NO:274 (see, e.g., FIG. 1).

In some cases, two sets of three complementarity-determining regions (CDRs) of an antigen binding fragment provided herein (e.g., SEQ ID NOs:1-3 and 9-11 or SEQ ID NOs:17-19 and 25-27) can be engineered into a CAR to create CAR⁺ cells (e.g., CAR⁺ T cells, CAR⁺ stem cells such as CAR⁺ induced pluripotent stem cells, or CAR⁺ natural killer (NK) cells) having the ability to target PSMA⁺ cells (e.g., PSMA⁺ tumor cells and/or PSMA⁺ tumor vasculature), can be engineered into an antibody structure that includes an Fc region to create antibodies having the ability to target PSMA⁺ cells (e.g., PSMA⁺ tumor cells and/or PSMA⁺ tumor vasculature) and induce antibody-dependent cell-mediated cytotoxicity (ADCC) against the target PSMA⁺ cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target PSMA⁺ cells (e.g., PSMA⁺ tumor cells and/or PSMA⁺ tumor vasculature) and induce one or more immune responses (e.g., T cell immune responses and/or ADCC using a cell engager in the absence of an Fc-containing antibody) against the target PSMA⁺ cells. It is noted that BiKE- and TriKE-mediated killing can be referred to as ADCC even though it is not initiated by an Fc domain.

In some cases, a single set of three CDRs of an antibody domain (e.g., a VH domain) provided herein (e.g., SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; SEQ ID NOs:89-91; or SEQ ID NOs:97-99) can be engineered into a CAR to create CAR⁺ cells (e.g., CAR⁺ T cells, CAR⁺ stem cells such as CAR⁺ induced pluripotent stem cells, or CAR⁺ NK cells) having the ability to target PSMA cells (e.g., PSMA⁺ tumor cells and/or PSMA⁺ tumor vasculature), can be engineered into an antibody structure that includes an Fc region to create antibodies having the ability to target PSMA⁺ cells (e.g., PSMA tumor cells and/or PSMA⁺ tumor vasculature) and induce ADCC against the target PSMA cells, and/or can be engineered into a cell engager such as a bi-specific T cell engager (e.g., a BiTE), a bi-specific killer engager (e.g., a BiKE), and/or a tri-specific killer engager (e.g., a TriKE) to create cell engagers having the ability to target PSMA cells (e.g., PSMA⁺ tumor cells and/or PSMA tumor vasculature) and induce one or more immune responses (e.g., T cell immune responses and/or ADCC using a cell engager in the absence of an Fc-containing antibody) against the target PSMA cells.

In addition, as described herein, binders (e.g., one or more antibodies, one or more antigen binding fragments, and/or one or more antibody domains) provided herein can be used to create conjugates that include the binder and a drug. For example, ADCs such as full antibody-drug conjugates, Fab-drug conjugates, scFv-drug conjugates, and/or antibody domain-drug conjugates can be designed to include an appropriate binder provided herein to create the conjugate. Such conjugates can be used to deliver the drug payload to target cells such as cancer cells (e.g., PSMA⁺ cancer cells) or cancer vasculature (e.g., PSMA⁺ cancer vasculature).

As also described herein, binders (e.g., one or more antibodies, one or more antigen binding fragments, one or more antibody domains, one or more cell engagers, and/or one or more ADCs) provided herein can be used to treat a mammal (e.g., a human) having cancer. For example, a mammal (e.g., a human) having cancer (e.g., a PSMA⁺ cancer) can be administered a composition comprising one or more binders (e.g., one or more antibodies, one or more antigen binding fragments, one or more antibody domains, one or more cell engagers, and/or one or more ADCs) described herein to reduce the number of cancer cells within the mammal, to induce ADCC against cancer cells within the mammal, and/or to increase the survival duration of the mammal from cancer.

As also described herein, cells (e.g., host cells) can be designed to express one or more binders (e.g., antibodies, antigen binding fragments, antibody domains, CARs, or cell engagers) having the ability to bind to a PSMA polypeptide. For example, cells such as T cells (e.g., CTLs), stem cells (e.g., induced pluripotent stem cells), or NK cells can be engineered to express one or more CARs having the ability to bind to a PSMA polypeptide. Such cells (e.g., PSMA-specific CAR⁺ T cells or NK cells) can be used to treat cancer.

In some cases, a binder (e.g., an antibody, antigen binding fragment, and/or antibody domain) provided herein can be used to detect the presence or absence of a PSMA polypeptide. For example, a binder (e.g., an antibody, antigen binding fragment, and/or antibody domain) provided herein can be used to determine whether or not a sample (e.g., a biological sample such tumor biopsy) obtained from a mammal (e.g., a human) contains PSMA⁺ cells (e.g., PSMA⁺ cancer cells). Having the ability to detect the presence or absence of a PSMA polypeptide (e.g., PSMA⁺ cancer cells) can allow clinicians, health professionals, and patients to make better decisions about possible treatment options. For example, detection of PSMA⁺ cancer cells within a mammal can allow clinicians, health professionals, and patients to select an appropriate anti-cancer treatment that targets the PSMA⁺ cancer cells. Such treatments that target the PSMA⁺ cancer cells can include administration of an anti-PSMA antibody such as J591 (see, e.g., Clinical Trial NCT04946370), MDX1201 (see, e.g., Clinical Trial NCT02048150), huJ591 (see, e.g., Clinical Trial NCT02552394), BAY 2315158 (see, e.g., Clinical Trial NCT03724747), and 10B3 (see, e.g., Clinical Trial NCT04496674) and/or one or more of the binders described herein having the ability to bind to a PSMA polypeptide and/or administration of one or more cells (e.g., PSMA-specific CAR⁺ T cells or NK cells) designed to express a binder described herein.

In general, one aspect of this document features an antibody comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions); (iv) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (v) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (vi) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (viii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (ix) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); (x) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); or (xi) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions). The antibody can comprise the ability to bind to SEQ ID NO:273 or SEQ ID NO:274. The antibody can comprise the heavy chain variable domain or region of the (i). The heavy chain variable domain or region can comprise an amino acid sequence having at least 90 percent identity to the amino acid sequence set forth in SEQ ID NO:8. The antibody can comprise the light chain variable domain or region of the (i). The light chain variable domain or region can comprise an amino acid sequence having at least 90 percent identity to the amino acid sequence set forth in SEQ ID NO:16. The antibody can comprise the heavy chain variable domain or region of the (ii). The heavy chain variable domain or region can comprise an amino acid sequence having at least 90 percent identity to the amino acid sequence set forth in SEQ ID NO:24. The antibody can comprise the light chain variable domain or region of the (ii). The light chain variable domain or region can comprise an amino acid sequence having at least 90 percent identity to the amino acid sequence set forth in SEQ ID NO:32. The antibody can comprise the heavy chain variable domain or region of the (iii). The heavy chain variable domain or region can comprise an amino acid sequence having at least 90 percent identity to the amino acid sequence set forth in SEQ ID NO:40. The antibody can comprise the heavy chain variable domain or region of the (iv). The heavy chain variable domain or region can comprise an amino acid sequence having at least 90 percent identity to the amino acid sequence set forth in SEQ ID NO:48. The antibody can comprise the heavy chain variable domain or region of the (v). The heavy chain variable domain or region can comprise an amino acid sequence having at least 90 percent identity to the amino acid sequence set forth in SEQ ID NO:56. The antibody can comprise the heavy chain variable domain or region of the (vi). The heavy chain variable domain or region can comprise an amino acid sequence having at least 90 percent identity to the amino acid sequence set forth in SEQ ID NO:64. The antibody can comprise the heavy chain variable domain or region of the (vii). The heavy chain variable domain or region can comprise an amino acid sequence having at least 90 percent identity to the amino acid sequence set forth in SEQ ID NO:72. The antibody can comprise the heavy chain variable domain or region of the (viii). The heavy chain variable domain or region can comprise an amino acid sequence having at least 90 percent identity to the amino acid sequence set forth in SEQ ID NO:80. The antibody can comprise the heavy chain variable domain or region of the (ix). The heavy chain variable domain or region can comprise an amino acid sequence having at least 90 percent identity to the amino acid sequence set forth in SEQ ID NO:88. The antibody can comprise the heavy chain variable domain or region of the (x). The heavy chain variable domain or region can comprise an amino acid sequence having at least 90 percent identity to the amino acid sequence set forth in SEQ ID NO:96. The antibody can comprise the heavy chain variable domain or region of the (xi). The heavy chain variable domain or region can comprise an amino acid sequence having at least 90 percent identity to the amino acid sequence set forth in SEQ ID NO:104. The antibody can be a monoclonal antibody. The antibody can be an scFv antibody. This paragraph is referred to as first aspect paragraph.

In another aspect, this document features an antigen binding fragment comprising (or consisting essentially of, or consisting of): (i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions); (ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions); (iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions); (iv) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (v) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (vi) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (vii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (viii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (ix) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); (x) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); or (xi) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions). The antigen binding fragment can comprise the ability to bind to SEQ ID NO:273 or SEQ ID NO:274. The antigen binding fragment can comprise the heavy chain variable domain or region of the (i). The heavy chain variable domain or region can comprise an amino acid sequence having at least 90 percent identity to the amino acid sequence set forth in SEQ ID NO:8. The antigen binding fragment can comprise the light chain variable domain or region of the (i). The light chain variable domain or region can comprise an amino acid sequence having at least 90 percent identity to the amino acid sequence set forth in SEQ ID NO:16. The antigen binding fragment can comprise the heavy chain variable domain or region of the (ii). The heavy chain variable domain or region can comprise an amino acid sequence having at least 90 percent identity to the amino acid sequence set forth in SEQ ID NO:24. The antigen binding fragment can comprise the light chain variable domain or region of the (ii). The light chain variable domain or region can comprise an amino acid sequence having at least 90 percent identity to the amino acid sequence set forth in SEQ ID NO:32. The antigen binding fragment can comprise the heavy chain variable domain or region of the (iii). The heavy chain variable domain or region can comprise an amino acid sequence having at least 90 percent identity to the amino acid sequence set forth in SEQ ID NO:40. The antigen binding fragment can comprise the heavy chain variable domain or region of the (iv). The heavy chain variable domain or region can comprise an amino acid sequence having at least 90 percent identity to the amino acid sequence set forth in SEQ ID NO:48. The antigen binding fragment can comprise the heavy chain variable domain or region of the (v). The heavy chain variable domain or region can comprise an amino acid sequence having at least 90 percent identity to the amino acid sequence set forth in SEQ ID NO:56. The antigen binding fragment can comprise the heavy chain variable domain or region of the (vi). The heavy chain variable domain or region can comprise an amino acid sequence having at least 90 percent identity to the amino acid sequence set forth in SEQ ID NO:64. The antigen binding fragment can comprise the heavy chain variable domain or region of the (vii). The heavy chain variable domain or region can comprise an amino acid sequence having at least 90 percent identity to the amino acid sequence set forth in SEQ ID NO:72. The antigen binding fragment can comprise the heavy chain variable domain or region of the (viii). The heavy chain variable domain or region can comprise an amino acid sequence having at least 90 percent identity to the amino acid sequence set forth in SEQ ID NO:80. The antigen binding fragment can comprise the heavy chain variable domain or region of the (ix). The heavy chain variable domain or region can comprise an amino acid sequence having at least 90 percent identity to the amino acid sequence set forth in SEQ ID NO:88. The antigen binding fragment can comprise the heavy chain variable domain or region of the (x). The heavy chain variable domain or region can comprise an amino acid sequence having at least 90 percent identity to the amino acid sequence set forth in SEQ ID NO:96. The antigen binding fragment can comprise the heavy chain variable domain or region of the (xi). The heavy chain variable domain or region can comprise an amino acid sequence having at least 90 percent identity to the amino acid sequence set forth in SEQ ID NO:104. The antigen binding fragment can be monoclonal. The antigen binding fragment can be an Fab. This paragraph is referred to as second aspect paragraph.

In another aspect, this document features an antibody domain comprising (or consisting essentially of, or consisting of): (i) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions); (ii) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions); (iii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions); (iv) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions); (v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions); (vi) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions); (vii) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions); (viii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); or (ix) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions). The antibody domain can comprise the ability to bind to SEQ ID NO:273 or SEQ ID NO:274. The antibody domain can comprise the heavy chain variable domain or region of the (i). The heavy chain variable domain or region can comprise an amino acid sequence having at least 90 percent identity to the amino acid sequence set forth in SEQ ID NO:40. The antibody domain can comprise the heavy chain variable domain or region of the (ii). The heavy chain variable domain or region can comprise an amino acid sequence having at least 90 percent identity to the amino acid sequence set forth in SEQ ID NO:48. The antibody domain can comprise the heavy chain variable domain or region of the (iii). The heavy chain variable domain or region can comprise an amino acid sequence having at least 90 percent identity to the amino acid sequence set forth in SEQ ID NO:56. The antibody domain can comprise the heavy chain variable domain or region of the (iv). The heavy chain variable domain or region can comprise an amino acid sequence having at least 90 percent identity to the amino acid sequence set forth in SEQ ID NO:64. The antibody domain can comprise the heavy chain variable domain or region of the (v). The heavy chain variable domain or region can comprise an amino acid sequence having at least 90 percent identity to the amino acid sequence set forth in SEQ ID NO:72. The antibody domain can comprise the heavy chain variable domain or region of the (vi). The heavy chain variable domain or region can comprise an amino acid sequence having at least 90 percent identity to the amino acid sequence set forth in SEQ ID NO:80. The antibody domain can comprise the heavy chain variable domain or region of the (vii). The heavy chain variable domain or region can comprise an amino acid sequence having at least 90 percent identity to the amino acid sequence set forth in SEQ ID NO:88. The antibody domain can comprise the heavy chain variable domain or region of the (viii). The heavy chain variable domain or region can comprise an amino acid sequence having at least 90 percent identity to the amino acid sequence set forth in SEQ ID NO:96. The antibody domain can comprise the heavy chain variable domain or region of the (ix). The heavy chain variable domain or region can comprise an amino acid sequence having at least 90 percent identity to the amino acid sequence set forth in SEQ ID NO:104. The antibody domain can be monoclonal. The antibody domain can be a VH domain. This paragraph is referred to as third aspect paragraph.

In another aspect, this document features a chimeric antigen receptor comprising (or consisting essentially of, or consisting of) an antigen binding domain, a hinge, a transmembrane domain, and one or more signaling domains, wherein the antigen binding domain comprises any antibody, any antigen-binding fragment, or any antibody domain of first aspect paragraph, second aspect paragraph, or third aspect paragraph. The antigen binding domain can comprise a scFv having the ability to bind to a PSMA polypeptide. The antigen binding domain can comprise a VH domain having the ability to bind to a PSMA polypeptide. The hinge can comprise a hinge set forth in FIG. 26. The transmembrane domain can comprise a transmembrane domain set forth in FIG. 27. The chimeric antigen receptor can comprise one or more signaling domains set forth in FIG. 28. This paragraph is referred to as fourth aspect paragraph.

In another aspect, this document features a cell engager comprising (or consisting essentially of, or consisting of) a first antigen binding domain, a linker, and a second antigen binding domain, wherein the first antigen binding domain comprises any antibody, any antigen-binding fragment, or any antibody domain of first aspect paragraph, second aspect paragraph, or third aspect paragraph. The first antigen binding domain can comprise a scFv having the ability to bind to a PSMA polypeptide. The first antigen binding domain can comprise a VH domain having the ability to bind to a PSMA polypeptide. The first antigen binding domain can be an IgG having the ability to bind to a PSMA polypeptide. The linker can comprise a linker set forth in FIG. 22 or FIG. 26. The second antigen binding domain can bind to a polypeptide expressed on the surface of T cells. The polypeptide expressed on the surface of T cells can be a CD3 polypeptide. The second antigen binding domain can be an antigen binding domain set forth in FIG. 41. The second antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The second antigen binding domain can be an antigen binding domain set forth in FIG. 42. The cell engager can comprise a third antigen binding domain. The third antigen binding domain can bind to a polypeptide expressed on the surface of NK cells. The polypeptide expressed on the surface of NK cells can be a CD16a, NKG2A, NKG2D, NKp30, NKp44, or NKp46 polypeptide. The third antigen binding domain can be an antigen binding domain set forth in FIG. 42. This paragraph is referred to as fifth aspect paragraph.

In another aspect, this document features an antibody-drug conjugate (ADC) comprising (or consisting essentially of, or consisting of) an antigen binging domain covalently linked to a drug, wherein the antigen binging domain comprises any antibody, any antigen-binding fragment, or any antibody domain of first aspect paragraph, second aspect paragraph, or third aspect paragraph. The antigen binding domain can comprise a scFv having the ability to bind to a PSMA polypeptide. The antigen binding domain can comprise a VH domain having the ability to bind to a PSMA polypeptide. The antigen binding domain can be an IgG having the ability to bind to a PSMA polypeptide. The drug can be selected from the group consisting of auristatins, mertansine, or pyrrolobenzodiazepine (PBD) dimers. This paragraph is referred to as sixth aspect paragraph.

In another aspect, this document features a composition comprising (or consisting essentially of, or consisting of) any antibody, any antigen-binding fragment, or any antibody domain of first aspect paragraph, second aspect paragraph, or third aspect paragraph. The composition can comprise any antibody of first aspect paragraph. The composition can comprise any antigen binding fragment of second aspect paragraph. The composition can comprise any antibody domain of third aspect paragraph.

In another aspect, this document features a composition comprising (or consisting essentially of, or consisting of) any cell engager of fifth aspect paragraph.

In another aspect, this document features a nucleic acid comprising (or consisting essentially of, or consisting of) a nucleic acid sequence encoding at least part of any antibody, any antigen-binding fragment, or any antibody domain of first aspect paragraph, second aspect paragraph, or third aspect paragraph. The nucleic acid sequence can encode the heavy chain variable domain or region of any one of (i)-(ii) of first aspect paragraph. The nucleic acid sequence can encode the light chain variable domain or region of any one of (i)-(ii) of first aspect paragraph. The nucleic acid sequence can encode the heavy chain variable domain or region of any one of (iii)-(xi) of first aspect paragraph. The nucleic acid can be a viral vector. The nucleic acid can be a phagemid.

In another aspect, this document features a nucleic acid comprising (or consisting essentially of, or consisting of) a nucleic acid sequence encoding any chimeric antigen receptor of fourth aspect paragraph or any cell engager of fifth aspect paragraph. The nucleic acid can be a viral vector. The nucleic acid can be a phagemid.

In another aspect, this document features a host cell comprising any nucleic acid of the preceding two paragraphs.

In another aspect, this document features a host cell that expresses any chimeric antigen receptor of fourth aspect paragraph or any cell engager of fifth aspect paragraph. The host cell can be a T cell, stem cell, or NK cell.

In another aspect, this document features a cell comprising any chimeric antigen receptor of fourth aspect paragraph. The cell can be a T cell, a stem cell, or an NK cell.

In another aspect, this document features a composition comprising (or consisting essentially of, or consisting of) any cell of the preceding three paragraphs.

In another aspect, this document features a composition comprising (or consisting essentially of, or consisting of) any ADC of sixth aspect paragraph. The composition can comprise a checkpoint inhibitor. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab.

In another aspect, this document features a method of treating a mammal having cancer. The method comprises (or consists essentially of, or consists of) administering, to the mammal, any composition of the preceding two paragraphs. The mammal can be a human. The cancer can be a PSMA⁺ cancer. The PSMA⁺ cancer can be selected from the group consisting of PSMA⁺ lung cancer, PSMA prostate cancer, PSMA⁺ colorectal cancer, PSMA⁺ renal cancer, PSMA⁺ bladder cancer, PSMA⁺ brain cancer, PSMA⁺ skin cancer, PSMA⁺ adrenal cancer, PSMA⁺ gastric cancer, PSMA⁺ pancreatic cancer, PSMA⁺ gynecologic cancer, PSMA⁺ sarcomas, and PSMA⁺ breast cancer. The number of cancer cells within the mammal can be reduced following the administering step.

In another aspect, this document features a method of treating a mammal having cancer. The method comprises (or consists essentially of, or consists of) (a) administering, to the mammal, any composition of the two preceding paragraphs referenced in the preceding paragraph, and (b) administering, to the mammal, a composition comprising a checkpoint inhibitor. The mammal can be a human. The cancer can be a PSMA⁺ cancer. The PSMA⁺ cancer can be selected from the group consisting of PSMA⁺ lung cancer, PSMA⁺ prostate cancer, PSMA⁺ colorectal cancer, PSMA⁺ renal cancer, PSMA⁺ bladder cancer, PSMA⁺ brain cancer, PSMA⁺ skin cancer, PSMA⁺ adrenal cancer, PSMA⁺ gastric cancer, PSMA⁺ pancreatic cancer, PSMA⁺ gynecologic cancer, PSMA⁺ sarcomas, and PSMA⁺ breast cancer. The checkpoint inhibitor can be selected from the group consisting of cemiplimab, nivolumab, pembrolizumab, JTX-4014, spartalizumab, camrelizumab, sintilimab, tislelizumab, toripalimab, dostarlimab, INCMGA00012, AMP-224, AMP-514, avelumab, durvalumab, atezolizumab, KN035, CK-301, AUNP12, CA-170, BMS-986189, and ipilimumab. The number of cancer cells within the mammal can be reduced following the administering steps (a) and (b).

In another aspect, this document features a method for binding a binding molecule to a PSMA polypeptide. The method comprises (or consists essentially of, or consists of) contacting the PSMA polypeptide with any antibody, any antigen binding fragment, or any antibody domain of first aspect paragraph, second aspect paragraph, or third aspect paragraph. The contacting can be performed in vitro. The contacting can be performed in vivo. The contacting can be performed within a mammal by administering the antibody, the antigen binding fragment, or the antibody domain to the mammal. The mammal can be a human.

In another aspect, this document features a method for binding a binding molecule to a PSMA polypeptide. The method comprises (or consists essentially of, or consists of) contacting the PSMA polypeptide with any chimeric antigen receptor of fourth aspect paragraph, any cell engager of fifth aspect paragraph, or any ADC of sixth aspect paragraph. The contacting can be performed in vitro. The contacting can be performed in vivo. The contacting can be performed within a mammal by administering the chimeric antigen receptor, the cell engager, or the ADC to the mammal. The mammal can be a human.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts amino acid residues 1 to 750 of a human PSMA polypeptide (SEQ ID NO:273). The underlined and bolded amino acid sequence (residues 44 to 750) of this human PSMA polypeptide was used to identify PSMA binders (SEQ ID NO:274).

FIGS. 2A and 2B depict the amino acid sequences of the heavy chain variable domain (FIG. 2A), linker (FIG. 2A), and the light chain variable domain (FIG. 2B) of a scFv designated Clone #1. The CDRs and framework sequences of each also are provided and delineated.

FIGS. 3A and 3B depict the amino acid sequences of the heavy chain variable domain (FIG. 3A), linker (FIG. 3A), and the light chain variable domain (FIG. 3B) of a scFv designated Clone #2. The CDRs and framework sequences of each also are provided and delineated.

FIG. 4 depicts the amino acid sequence of a VH domain designated Clone #3. The CDRs and framework sequences also are delineated.

FIG. 5 depicts the amino acid sequence of a VH domain designated Clone #4. The CDRs and framework sequences also are delineated.

FIG. 6 depicts the amino acid sequence of a VH domain designated Clone #5. The CDRs and framework sequences also are delineated.

FIG. 7 depicts the amino acid sequence of a VH domain designated Clone #6. The CDRs and framework sequences also are delineated.

FIG. 8 depicts the amino acid sequence of a VH domain designated Clone #7. The CDRs and framework sequences also are delineated.

FIG. 9 depicts the amino acid sequence of a VH domain designated Clone #8. The CDRs and framework sequences also are delineated.

FIG. 10 depicts the amino acid sequence of a VH domain designated Clone #9. The CDRs and framework sequences also are delineated.

FIG. 11 depicts the amino acid sequence of a VH domain designated Clone #10. The CDRs and framework sequences also are delineated.

FIG. 12 depicts the amino acid sequence of a VH domain designated Clone #11. The CDRs and framework sequences also are delineated.

FIG. 13 depicts the nucleic acid sequences encoding the indicated chains/domains of Clones #1-#11.

FIG. 14 depicts the structure of an exemplary Ig and provides the amino acid and nucleic acid sequences of an exemplary hinge, CH2, and CH3 regions/domains.

FIG. 15 depicts the structure of exemplary scFv's.

FIGS. 16A and 16B depict the amino acid sequences of an exemplary heavy chain variable domain (FIG. 16A) and an exemplary light chain variable domain (FIG. 16B) of an exemplary scFv. The CDRs and framework sequences of each also are delineated. An exemplary linker amino acid sequence such as a linker amino acid sequence set forth in FIG. 22 can be used to link the heavy chain variable domain and the light chain variable domain together to form a scFv.

FIGS. 17A and 17B depict the amino acid sequences of an exemplary heavy chain variable domain (FIG. 17A) and an exemplary light chain variable domain (FIG. 17B) of an exemplary scFv. The CDRs and framework sequences of each also are delineated. An exemplary linker amino acid sequence such as a linker amino acid sequence set forth in FIG. 22 can be used to link the heavy chain variable domain and the light chain variable domain together to form a scFv.

FIGS. 18A and 18B depict the amino acid sequences of an exemplary heavy chain variable domain (FIG. 18A) and an exemplary light chain variable domain (FIG. 18B) of an exemplary scFv. The CDRs and framework sequences of each also are delineated. An exemplary linker amino acid sequence such as a linker amino acid sequence set forth in FIG. 22 can be used to link the heavy chain variable domain and the light chain variable domain together to form a scFv.

FIGS. 19A and 19B depict the amino acid sequences of an exemplary heavy chain variable domain (FIG. 19A) and an exemplary light chain variable domain (FIG. 19B) of an exemplary scFv. The CDRs and framework sequences of each also are delineated. An exemplary linker amino acid sequence such as a linker amino acid sequence set forth in FIG. 22 can be used to link the heavy chain variable domain and the light chain variable domain together to form a scFv.

FIGS. 20A and 20B depict the amino acid sequences of an exemplary heavy chain variable domain (FIG. 20A) and an exemplary light chain variable domain (FIG. 20B) of an exemplary scFv. The CDRs and framework sequences of each also are delineated. An exemplary linker amino acid sequence such as a linker amino acid sequence set forth in FIG. 22 can be used to link the heavy chain variable domain and the light chain variable domain together to form a scFv.

FIGS. 21A and 21B depict the amino acid sequences of an exemplary heavy chain variable domain (FIG. 21A) and an exemplary light chain variable domain (FIG. 21B) of an exemplary scFv. The CDRs and framework sequences of each also are delineated. An exemplary linker amino acid sequence such as a linker amino acid sequence set forth in FIG. 22 can be used to link the heavy chain variable domain and the light chain variable domain together to form a scFv.

FIG. 22 depicts exemplary linker amino acid sequences that can be used to link a heavy chain variable domain and a light chain variable domain together to form a scFv. These linker sequences also can be used to create CARs and cell engagers.

FIG. 23A depicts the structure of an exemplary CARs. FIG. 23B is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a scFv provided herein (e.g., a scFv designed to include two sets of three CDRs such as CDR1, CDR2, and CDR3 of a heavy chain and CDR1, CDR2, and CDR3 of a light chain (in either order) of an antigen binding fragment provided herein, for example, SEQ ID NOs:1-3 and 9-11 or SEQ ID NOs:17-19 and 25-27), followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ intracellular signaling domain sequence).

FIG. 24A depicts the structure of an exemplary CARs. FIG. 24B is a schematic of an exemplary CAR construct designed to express a CAR. A promotor sequence (e.g., a CMV immediate early promotor sequence) can be followed by a signal peptide sequence (e.g., a GM-CSF signal peptide sequence), followed by a VH domain provided herein (e.g., a VH domain designed to include a set of three CDRs such as CDR1, CDR2, and CDR3 of a VH domain provided herein, for example, SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; SEQ ID NOs:89-91; or SEQ ID NOs:97-99), followed by an optional linker (not shown), followed by an optional hinge (e.g., a CD8 hinge sequence; not shown), followed by a transmembrane sequence (e.g., a CD8 transmembrane sequence), followed by one or more intracellular signaling domain sequences (e.g., a 4-1BB (CD137) intracellular signaling domain sequence and a CD3ζ (intracellular signaling domain sequence).

FIG. 25 depicts the amino acid sequences of exemplary signal peptides that can be used to design a CAR.

FIG. 26 depicts the amino acid sequences of exemplary hinges that can be used to design a CAR.

FIG. 27 depicts the amino acid sequences of exemplary transmembrane domains that can be used to design a CAR.

FIG. 28 depicts the amino acid sequences of exemplary intracellular signaling domains that can be used to design a CAR.

FIG. 29 depicts an amino acid sequence of a CAR (CAR #1) designed to include a scFv of Clone #1 and a nucleic acid sequence encoding that CAR. The various components of this CAR (e.g., domains and linkers) are provided and delineated.

FIG. 30 depicts an amino acid sequence of a CAR (CAR #2) designed to include a scFv of Clone #2 and a nucleic acid sequence encoding that CAR. The various components of this CAR (e.g., domains and linkers) are provided and delineated.

FIG. 31 depicts an amino acid sequence of a CAR (CAR #3) designed to include a VH domain of Clone #3 and a nucleic acid sequence encoding that CAR. The various components of this CAR (e.g., domains and linkers) are provided and delineated.

FIG. 32 depicts an amino acid sequence of a CAR (CAR #4) designed to include a VH domain of Clone #4 and a nucleic acid sequence encoding that CAR. The various components of this CAR (e.g., domains and linkers) are provided and delineated.

FIG. 33 depicts an amino acid sequence of a CAR (CAR #5) designed to include a VH domain of Clone #5 and a nucleic acid sequence encoding that CAR. The various components of this CAR (e.g., domains and linkers) are provided and delineated.

FIG. 34 depicts an amino acid sequence of a CAR (CAR #6) designed to include a VH domain of Clone #6 and a nucleic acid sequence encoding that CAR. The various components of this CAR (e.g., domains and linkers) are provided and delineated.

FIG. 35 depicts an amino acid sequence of a CAR (CAR #7) designed to include a VH domain of Clone #7 and a nucleic acid sequence encoding that CAR. The various components of this CAR (e.g., domains and linkers) are provided and delineated.

FIG. 36 depicts an amino acid sequence of a CAR (CAR #8) designed to include a VH domain of Clone #8 and a nucleic acid sequence encoding that CAR. The various components of this CAR (e.g., domains and linkers) are provided and delineated.

FIG. 37 depicts an amino acid sequence of a CAR (CAR #9) designed to include a VH domain of Clone #9 and a nucleic acid sequence encoding that CAR. The various components of this CAR (e.g., domains and linkers) are provided and delineated.

FIG. 38 depicts an amino acid sequence of a CAR (CAR #10) designed to include a VH domain of Clone #10 and a nucleic acid sequence encoding that CAR. The various components of this CAR (e.g., domains and linkers) are provided and delineated.

FIG. 39 depicts an amino acid sequence of a CAR (CAR #11) designed to include a VH domain of Clone #11 and a nucleic acid sequence encoding that CAR. The various components of this CAR (e.g., domains and linkers) are provided and delineated.

FIG. 40A is a schematic of an exemplary BiTE designed using CDR1, CDR2, and CDR3 of a heavy chain provided herein and CDR1, CDR2, and CDR3 of a light chain provided herein in an Ig format (e.g., an IgG1 format). A humanized anti-CD3 scFv (e.g., an gOKT3-7 scFv set forth in U.S. Pat. No. 6,750,325) can be linked to the C-terminus of the light chain via a linker (e.g., a (G₄S)₃ linker). FIG. 40B depicts an amino acid sequence of a linker sequence (SEQ ID NO:170; nucleic acid sequence of the linker is SEQ ID NO:275) followed by an gOKT3-7 scFv sequence, which can be attached to a light chain as shown in FIG. 40A. FIG. 40B also depicts a nucleic acid sequence encoding that linker and gOKT3-7 scFv. FIG. 40C depicts the amino acid sequence for an exemplary BiTE designed to include a scFv of Clone #1. The various components of this BiTE (e.g., domains and linkers) are provided and delineated, and a nucleic acid sequence encoding this exemplary BiTE is provided. FIG. 40D is a schematic of an exemplary BiTE design that includes a knob and hole configuration. FIG. 40E depicts the amino acid sequence for an exemplary BiTE designed to include a VH of Clone #4. The various components of this BiTE (e.g., domains and linkers) are provided and delineated, and a nucleic acid sequence encoding this exemplary BiTE is provided. FIG. 40F depicts the amino acid sequence for an exemplary BiTE designed to include a VH of Clone #9. The various components of this BiTE (e.g., domains and linkers) are provided and delineated, and a nucleic acid sequence encoding this exemplary BiTE is provided.

FIG. 41 depicts the amino acid sequences of exemplary antigen binding domains that can be used to design cell engagers that bind to T cells.

FIG. 42 depicts the amino acid sequences of exemplary antigen binding domains that can be used to design cell engagers that bind to NK cells.

FIG. 43 depicts the amino acid sequence for an exemplary BiKE (BiKE #1) designed to include a scFv of Clone #1. The various components of this BiKE (e.g., domains and linkers) are provided and delineated.

FIG. 44 depicts the amino acid sequence for an exemplary BiKE (BiKE #2) designed to include a scFv of Clone #2. The various components of this BiKE (e.g., domains and linkers) are provided and delineated.

FIG. 45 is a schematic of an exemplary tandem ADC format with a VH binder for PSMA and a small molecular weight binder for human serum albumin (HSA). The drug-payload can be conjugated to the C-terminal of the fused polypeptides.

FIG. 46 depicts the amino acid sequence that can be used to design an exemplary ADC designed to include a VH of Clone #4. The various polypeptide components for this ADC (e.g., domains and linkers) are provided and delineated, and a nucleic acid sequence encoding this exemplary ADC format is provided. The sortase tag at, for example, its C-terminal can be used for sortase-assisted conjugation of drug payloads.

FIG. 47A is a schematic of an exemplary VH binder. FIG. 47B is a graph plotting the binding of the indicated anti-PSMA single domain VH antibodies to LNCaP cells as compared to the binding of VH-585* and scFv-J591*. VH-Fla refers to anti-PSMA Clone #4. VH-4mut refers to anti-PSMA Clone #9. VH-Z4D2 refers to anti-PSMA Clone #3. VH-W5A4 refers to anti-PSMA Clone #6. VH-585* refers to an anti-PSMA VH clone of International PCT Patent Application Publication No. WO2020206330A. scFv-J591* refers to a scFv-J591 clone of U.S. Pat. No. 7,045,605.

FIG. 48A is a schematic of an exemplary BiTE that includes an antigen binding domain that binds to CD3 and an antigen binding domain that binds to PSMA. FIG. 48B is a graph plotting the binding of the indicated anti-PSMA BiTEs to PSMA⁺ PC3-PiP cells as compared to the binding of a BiTE designated TNB-BiTE-585*. TNB-BiTE-Fla refers to the anti-PSMA BiTE of FIG. 40E. TNB-BiTE-4mut refers to the anti-PSMA BiTE of FIG. 40F. TNB-BiTE-585* refers to an anti-PSMA BiTE of International PCT Patent Application Publication No. WO2020206330A.

FIG. 49A is a schematic of an exemplary BiTE that includes an antigen binding domain that binds to CD3 and an antigen binding domain that binds to PSMA. FIG. 49B is a graph plotting the cell killing of PSMA PC3-PiP cells and of PSMA⁻ DU145 cells by the indicated anti-PSMA BiTEs as compared to the cell killing of a BiTE designated BiTE-585. BiTE-F1a refers to the anti-PSMA BiTE of FIG. 40E. BiTE-4mut refers to the anti-PSMA BiTE of FIG. 40F. BiTE-585 refers to an anti-PSMA BiTE of International PCT Patent Application Publication No. WO2020206330A. Cell killing was detected using LDH-Glo cytotoxicity assay (Promega).

FIG. 50A is a schematic of an exemplary ADC that includes an antigen binding domain that binds to PSMA and a drug (e.g., SG3199, DM-1, or MMAE). FIG. 50B is a graph plotting the cell killing of PSMA PC3-PiP cells and of PSMA⁻ PC3 cells by the indicated ADCs. VH(F1a)-Fc-SG3199 refers to an ADC containing an anti-PSMA binder of Clone #4 and SG3199. VH(Ab8)-Fc-SG3199 is an isotype control ADC containing an anti-SARS-CoV2 antibody. FIG. 50C is a graph plotting the cell killing of PSMA PC3-PiP cells and of PSMA⁻ PC3 cells by the indicated ADCs. VH(F1a)-Fc-DM1 refers to an ADC containing an anti-PSMA binder of Clone #4 and DM-1. FIG. 50D is a graph plotting the cell killing of PSMA⁺ PC3-PiP cells and of PSMA⁻ PC3 cells by the indicated ADCs. VH(F1a)-Fc-MMAE refers to an ADC containing an anti-PSMA binder of Clone #4 and MMAE.

### DETAILED DESCRIPTION

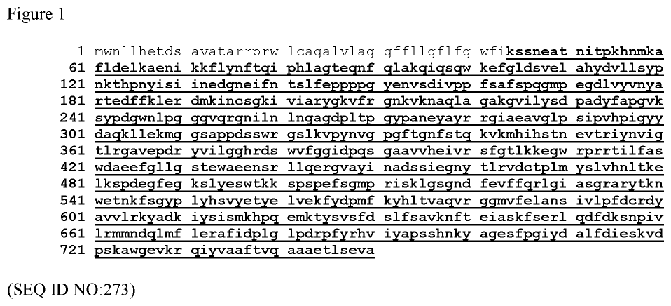

This document provides binders (e.g., antibodies, antigen binding fragments, antibody domains, CARs, cell engagers, and ADCs) that bind (e.g., specifically bind) to a PSMA polypeptide (e.g., a human PSMA polypeptide). For example, the document provides binders (e.g., antibodies, antigen binding fragments, antibody domains, CARs, cell engagers, and ADCs) that bind (e.g., specifically bind) to a polypeptide comprising, consisting essentially of, or consisting of the amino acid set forth in SEQ ID NO:273 or SEQ ID NO:274 (see, e.g., FIG. 1).

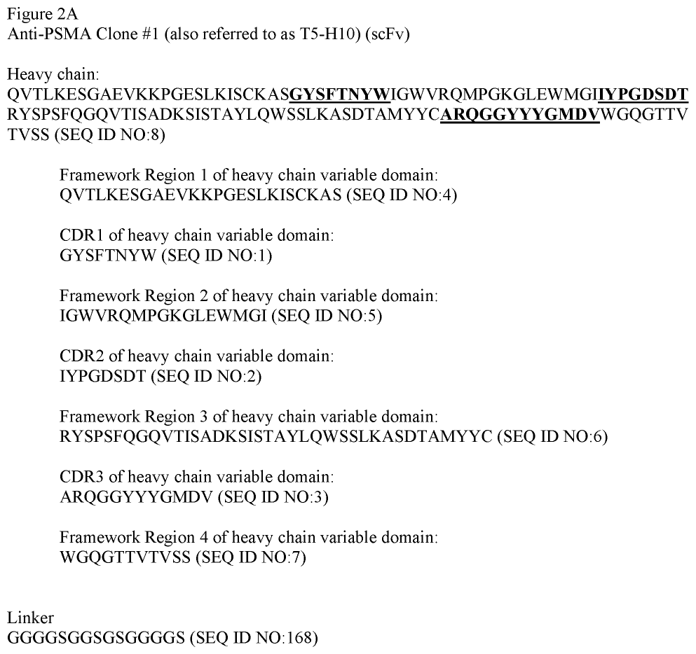

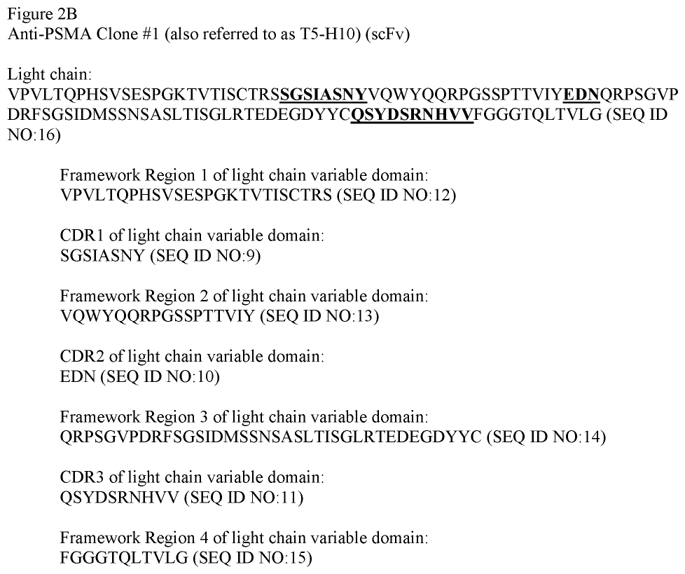

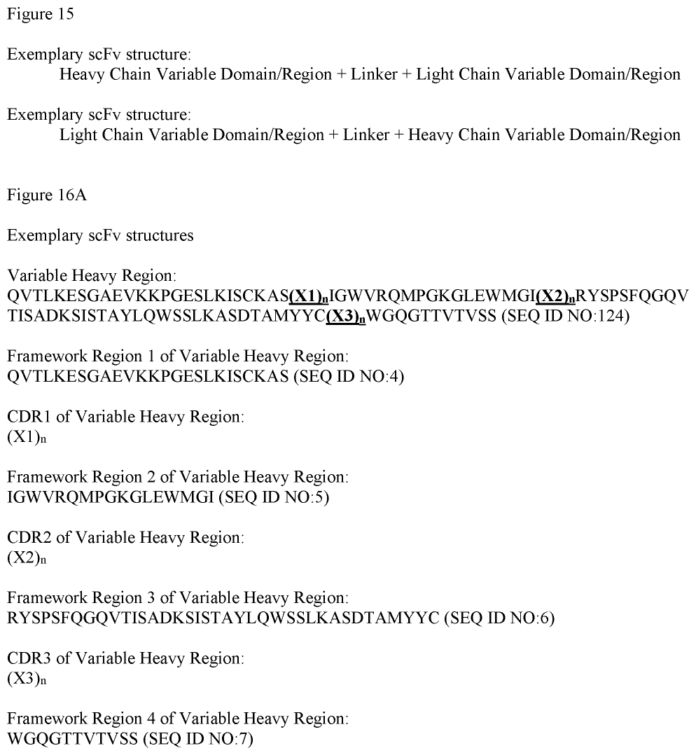

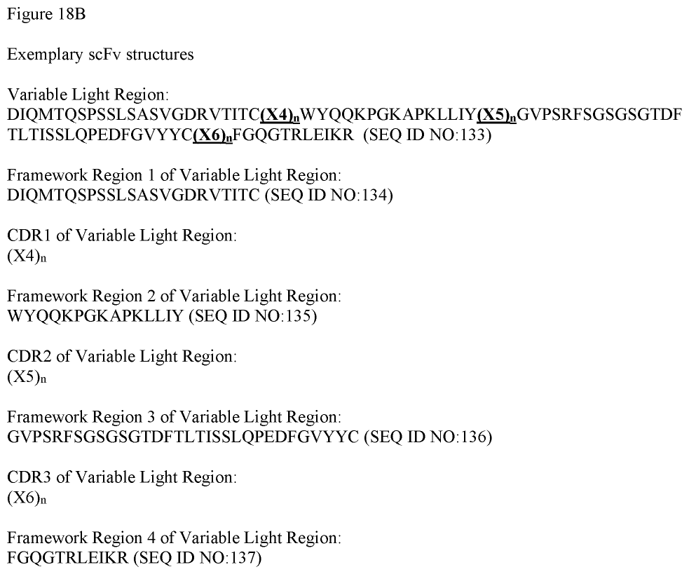

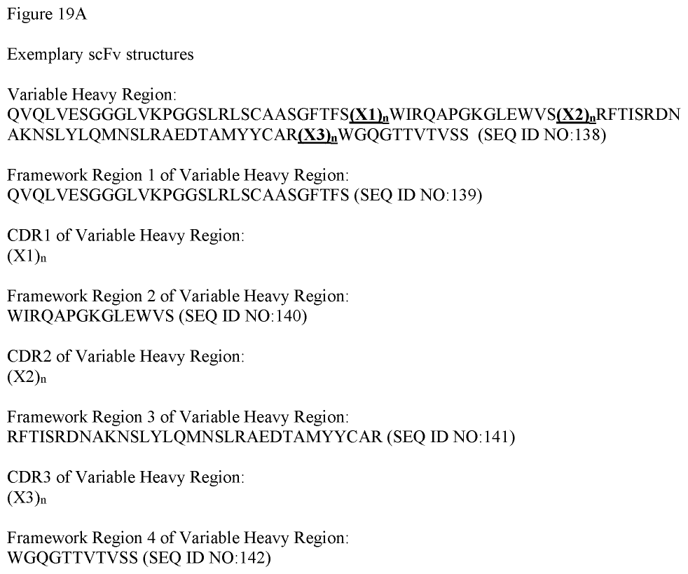

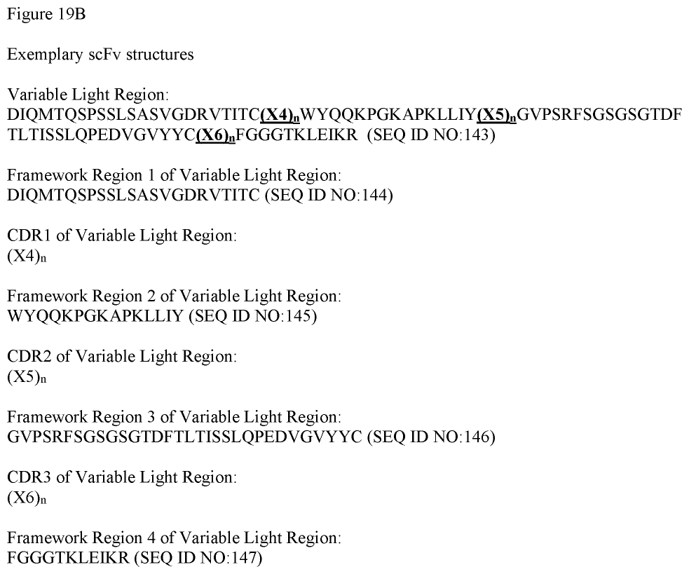

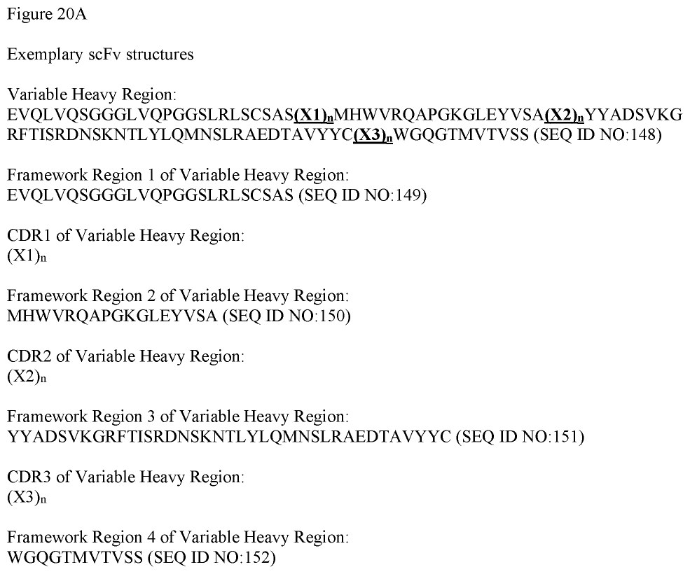

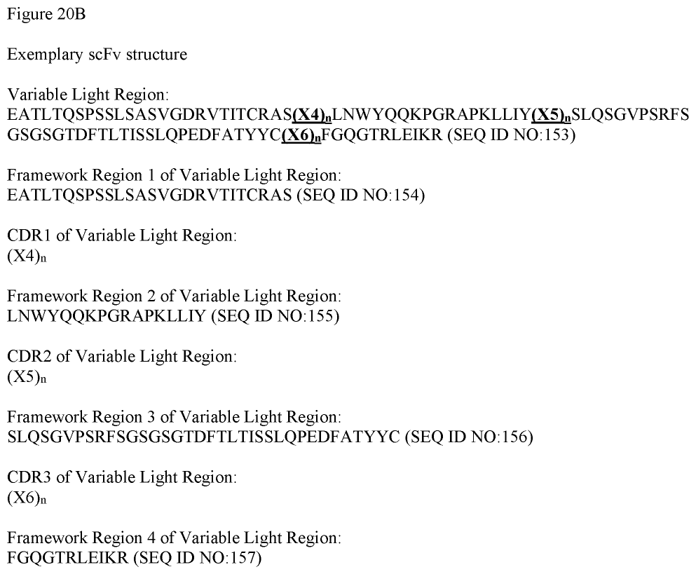

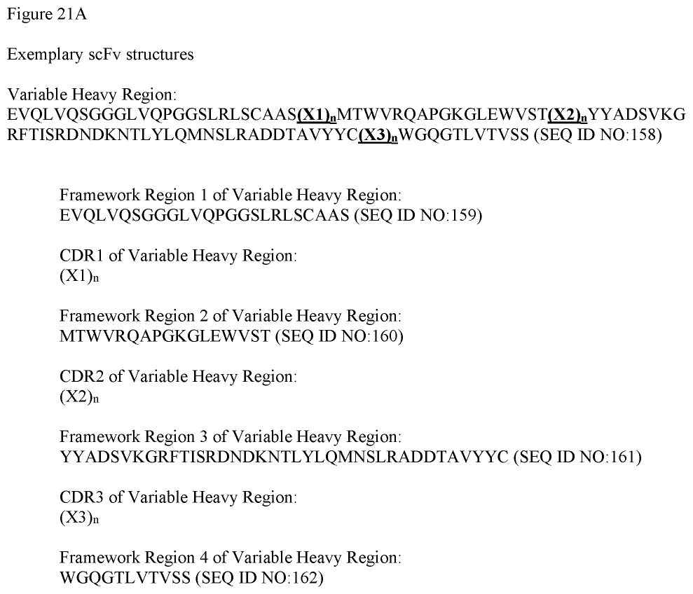

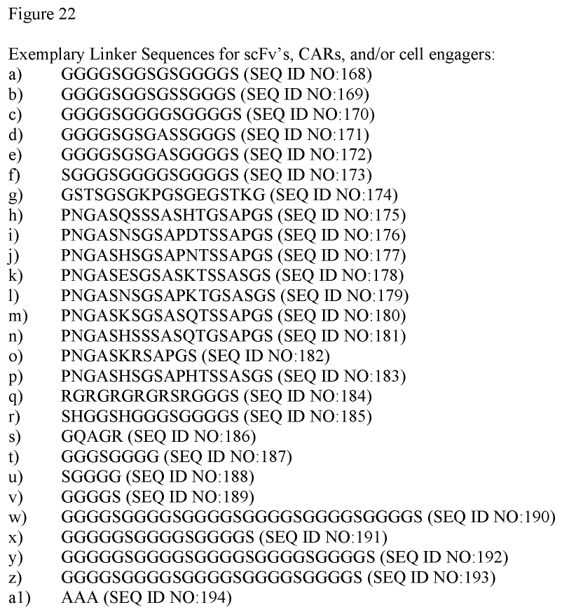

The term “antibody” as used herein includes polyclonal antibodies, monoclonal antibodies, recombinant antibodies, humanized antibodies, human antibodies, chimeric antibodies, multi-specific antibodies (e.g., bispecific antibodies) formed from at least two antibodies, diabodies, single-chain variable fragment antibodies (e.g., scFv antibodies), and tandem single-chain variable fragments antibody (e.g., taF_{V}). A diabody can include two chains, each having a heavy chain variable domain and a light chain variable domain, either from the same or from different antibodies (see, e.g., Hornig and Farber-Schwarz, Methods Mol. Biol., 907:713-27 (2012); and Brinkmann and Kontermann, MAbs., 9(2):182-212 (2017)). The two variable regions can be connected by a polypeptide linker (e.g., a polypeptide linker having five to ten residues in length or a polypeptide linker as set forth in FIG. 22). In some cases, an interdomain disulfide bond can be present in one or both of the heavy chain variable domain and light chain variable domain pairs of the diabody. A scFv is a single-chain polypeptide antibody in which the heavy chain variable domain and the light chain variable domain are directly connected or connected via a polypeptide linker (e.g., a polypeptide linker having eight to 18 residues in length or a polypeptide linker as set forth in FIG. 22). See, also, Chen et al., Adv. Drug Deliv. Rev., 65(10):1357-1369 (2013). A scFv can be designed to have an orientation with the heavy chain variable domain being followed by the light chain variable domain or can be designed to have an orientation with the light chain variable domain being followed by the heavy chain variable domain. In both cases, the optional linker can be located between the two domains. Examples of scFv structures of scFv's provided herein include, without limitation, those structures set forth in FIGS. 2A-2B, 3A-3B, 15, 16A-16B, 17A-17B, 18A-18B, 19A-19B, 20A-20B, and 21A-21B.

An antibody provided herein can include the CDRs as described herein (e.g., as described in Table 40) and can be configured to be a human antibody, a humanized antibody, or a chimeric antibody. In some cases, an antibody provided herein can include the CDRs as described herein (e.g., as described in Table 40) and can be a monoclonal antibody. In some cases, an antibody provided herein can include the CDRs as described herein (e.g., as described in Table 40) and can be configured as a scFv antibody.

The term “antigen binding fragment” as used herein refers to a fragment of an antibody (e.g., a fragment of a humanized antibody, a fragment of a human antibody, or a fragment of a chimeric antibody) having the ability to bind to an antigen. Examples of antigen binding fragments include, without limitation, Fab, Fab′, or F(ab′)₂ antigen binding fragments. An antigen binding fragment provided herein can include the CDRs as described herein (e.g., as described in Table 40) and can be configured to be a human antigen binding fragment, a humanized antigen binding fragment, or a chimeric antigen binding fragment. In some cases, an antigen binding fragment provided herein can include the CDRs as described herein (e.g., as described in Table 40) and can be a monoclonal antigen binding fragment. In some cases, an antigen binding fragment provided herein can include the CDRs as described herein (e.g., as described in Table 40) and can be configured as an Fab antibody. In some cases, a Fab antibody can include a partial hinge sequence (e.g., EPKSCDKT (SEQ ID NO:276)) for disulfide bonding between heavy and light chains of the Fab.

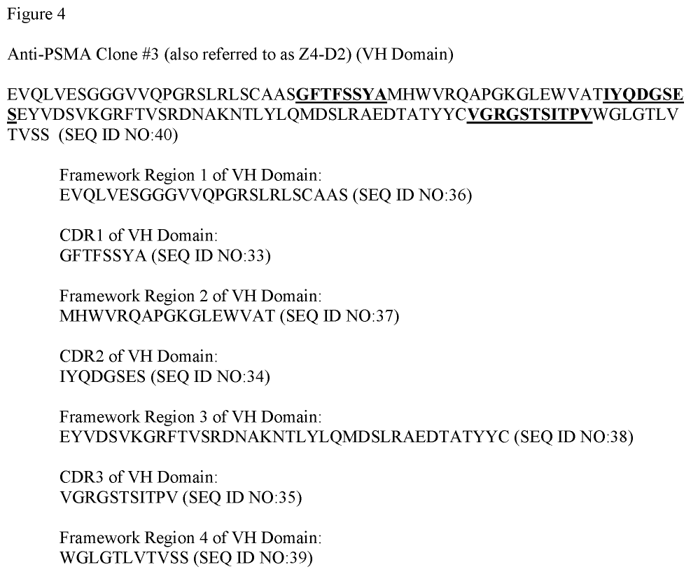

The term “antibody domain” as used herein refers to a domain of an antibody such as a heavy chain variable domain (VH domain) or a light chain variable domain (VL domain) in the absence of one or more other domains of an antibody. In some cases, an antibody domain can be a single antibody domain (e.g., a VH domain or a VL domain) having the ability to bind to an antigen. An antibody domain provided herein can include the CDRs as described herein (e.g., as described in Table 40) and can be a human antibody domain (e.g., a human VH domain), a humanized antibody domain (e.g., a humanized VH domain), or a chimeric antibody domain (e.g., a chimeric VH domain). In some cases, an antibody domain provided herein can include the CDRs as described herein (e.g., as described in Table 40) and can be a monoclonal antibody domain. In some cases, an antibody domain provided herein can include the CDRs as described herein (e.g., as described in Table 40) and can be engineered as a single VH domain or a single VL domain. Examples of VH domain's provided herein include, without limitation, those structures set forth in FIGS. 4-12.

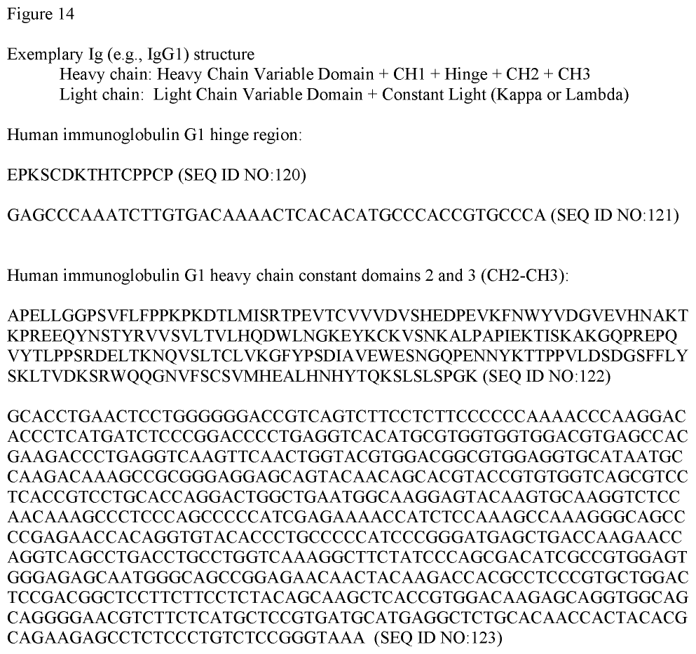

An anti-PSMA antibody, anti-PSMA antigen binding fragment, or anti-PSMA antibody domain provided herein can be of the IgA-, IgD-, IgE-, IgG-, or IgM-type, including IgG- or IgM-types such as, without limitation, IgG₁-, IgG₂-, IgG₃-, IgG₄-, IgM₁-, and IgM₂-types. In some cases, an antibody provided herein (e.g., an anti-PSMA antibody) can be a scFv antibody. In some cases, an antigen binding fragment provided herein (e.g., an anti-PSMA antibody fragment) can be an Fab. In some cases, an antibody provided herein (e.g., an anti-PSMA antibody) can be a fully intact antibody having the structure set forth in FIG. 14. In some cases, an antibody domain provided herein (e.g., an anti-PSMA antibody domain) can be a VH domain.

In some cases, an anti-PSMA antibody, anti-PSMA antigen binding fragment, or anti-PSMA antibody domain provided herein can compete with the J591 anti-PSMA antibody for binding to PSMA. Examples of such anti-PSMA antibodies, anti-PSMA antigen binding fragments, and anti-PSMA antibody domains include, without limitation, clones #1-#11 (see, e.g., FIGS. 2-12).

In some cases, an anti-PSMA antibody, anti-PSMA antigen binding fragment, or anti-PSMA antibody domain provided herein can be fully human. In some cases, an anti-PSMA antibody, anti-PSMA antigen binding fragment, or anti-PSMA antibody domain provided herein can have a low risk for inducing immunogenicity within a human.

The term “chimeric antigen receptor” as used herein refers to a chimeric polypeptide that is designed to include an optional signal peptide, an antigen binding domain, an optional hinge, a transmembrane domain, and one or more intracellular signaling domains. As described herein, the antigen binding domain of a CAR provided herein can be designed to bind to a PSMA polypeptide (e.g., a human PSMA polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a PSMA polypeptide (e.g., a human PSMA polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27). In some examples, a CAR provided herein can be designed to include an antigen binding domain that includes a single set of three CDRs (e.g., a CDR1, CDR2, and CDR3) of an antibody domain (e.g., a VH domain) provided herein (e.g., SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; SEQ ID NOs:89-91; or SEQ ID NOs:97-99). In some cases, an antigen binding domain of a CAR targeting a PSMA polypeptide can be designed to include a VH domain described herein or a scFv antibody described herein.

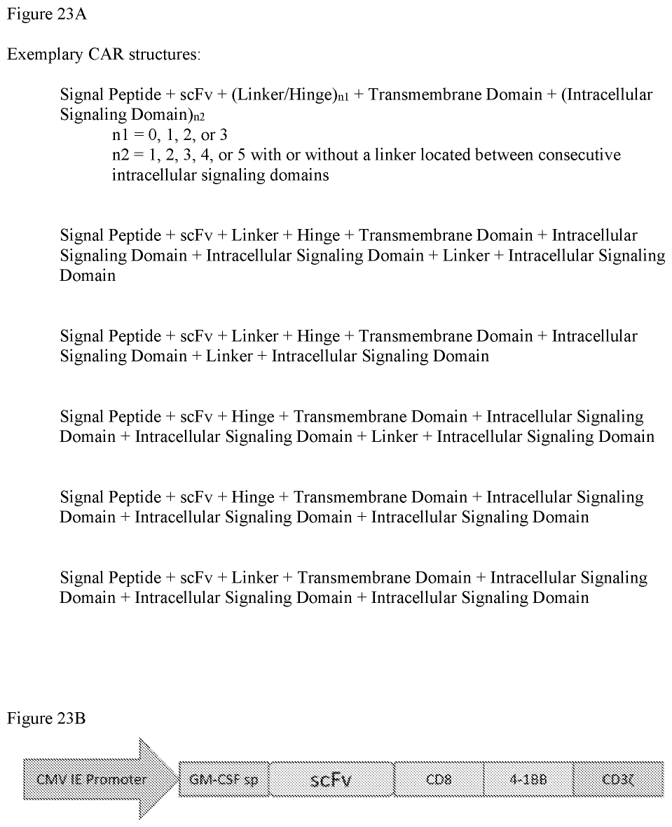

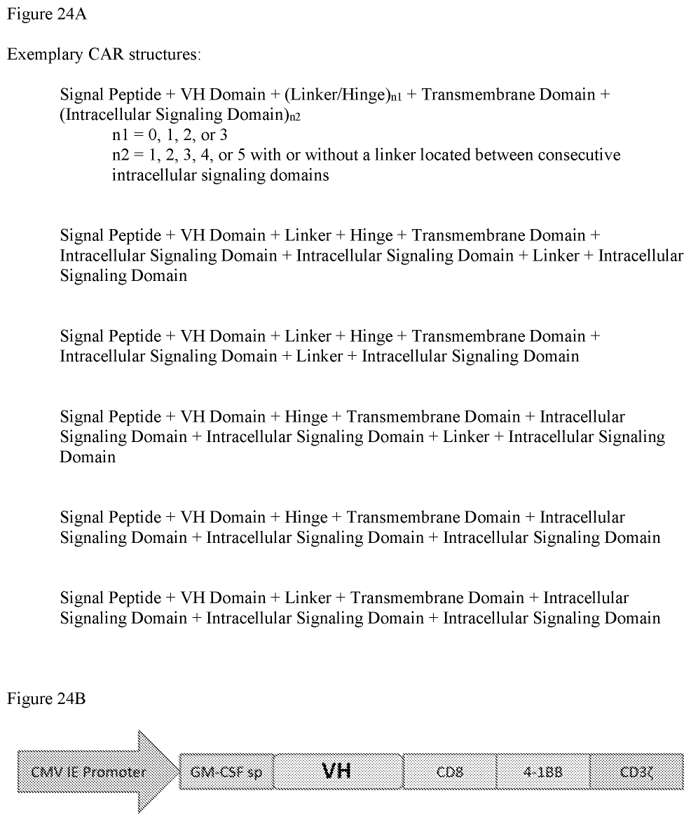

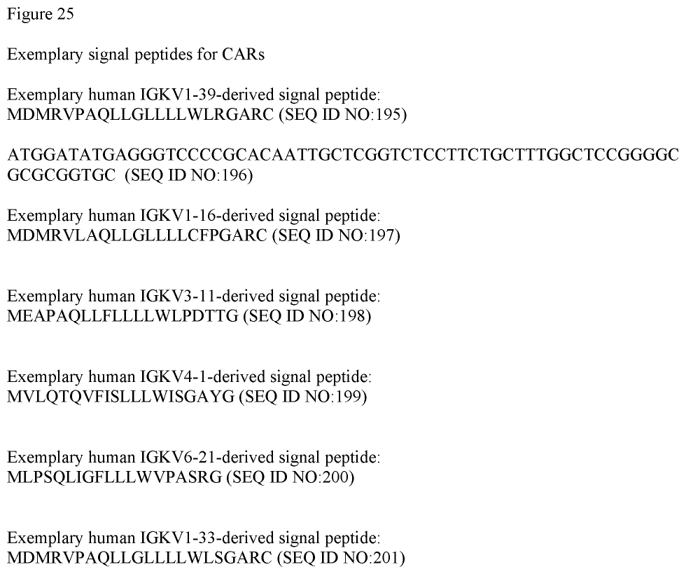

Examples of CAR structures that can be used to make a CAR provided herein include, without limitation, those set forth in FIGS. 23A, 23B, 24A, 24B, and 29-39. In some cases, a CAR provided herein can be designed to include a signal peptide. Any appropriate signal peptide can be used to design a CAR described herein. Examples of signal peptide that can be used to make a CAR described herein include without limitation, a human IGKV1-39-derived signal peptide, IGKV1-16, IGKV1-33, IGKV3-11, IGKV4-1, or IGKV6-21. In some cases, a CAR provided herein can be designed to include a signal peptide that comprises, consists essentially of, or consists of one of the amino acid sequences set forth in FIG. 25. In some cases, a CAR provided herein can be designed to include a signal peptide that comprises, consists essentially of, or consists of one of the amino acid sequences set forth in FIG. 25 with one, two, three, four, five, six, seven, eight, nine, or ten amino acid deletions, additions, substitutions, or combinations thereof. In some cases, a CAR provided herein can be designed to include a signal peptide that comprises, consists essentially of, or consists of one of the amino acid sequences set forth in FIG. 25 with two or less, three or less, four or less, five or less, six or less, seven or less, eight or less, nine or less, or ten or less amino acid deletions, additions, substitutions, or combinations thereof.

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. In some cases, a linker sequence can be used as a hinge to make a CAR described herein. For example, any one of the linker sequences set forth in FIG. 22 can be used as a hinge of a CAR described herein.

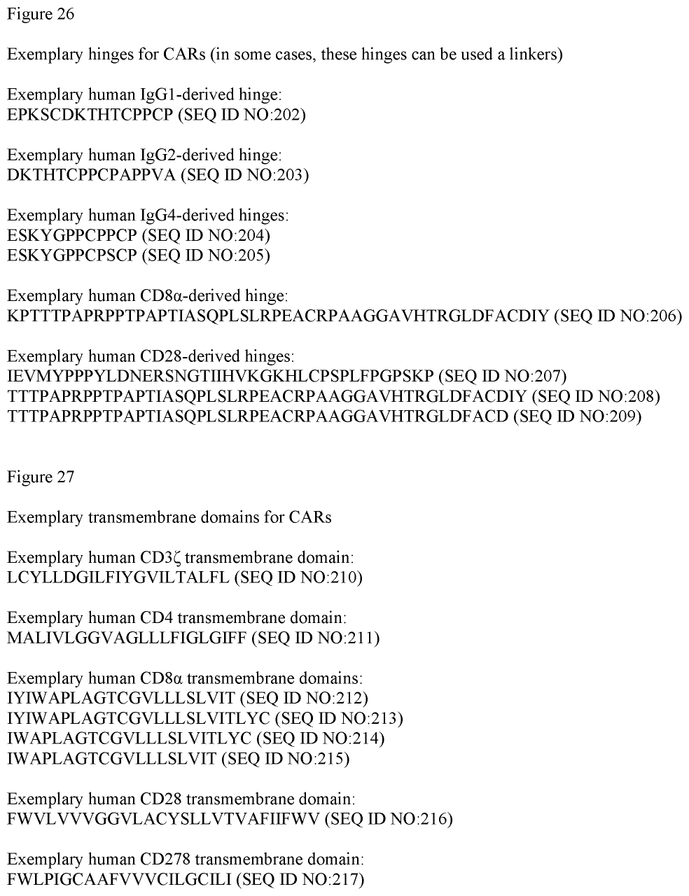

In some cases, a CAR provided herein can be designed to include a hinge that comprises, consists essentially of, or consists of one of the amino acid sequences set forth in FIG. 22 or FIG. 26. In some cases, a CAR provided herein can be designed to include a hinge that comprises, consists essentially of, or consists of one of the amino acid sequences set forth in FIG. 22 or FIG. 26 with one, two, three, four, five, six, seven, eight, nine, or ten amino acid deletions, additions, substitutions, or combinations thereof. In some cases, a CAR provided herein can be designed to include a hinge that comprises, consists essentially of, or consists of one of the amino acid sequences set forth in FIG. 22 or FIG. 26 with two or less, three or less, four or less, five or less, six or less, seven or less, eight or less, nine or less, or ten or less amino acid deletions, additions, substitutions, or combinations thereof.

A CAR provided herein can be designed to include any appropriate transmembrane domain. For example, the transmembrane domain of a CAR provided herein can be, without limitation, a CD3ζ transmembrane domain, a CD4 transmembrane domain, a CD8a transmembrane domain, a CD28 transmembrane domain, and a 4-1BB transmembrane domain. In some cases, a CAR provided herein can be designed to include a transmembrane domain that comprises, consists essentially of, or consists of one of the amino acid sequences set forth in FIG. 27. In some cases, a CAR provided herein can be designed to include a transmembrane domain that comprises, consists essentially of, or consists of one of the amino acid sequences set forth in FIG. 27 with one, two, three, four, five, six, seven, eight, nine, or ten amino acid deletions, additions, substitutions, or combinations thereof. In some cases, a CAR provided herein can be designed to include a transmembrane domain that comprises, consists essentially of, or consists of one of the amino acid sequences set forth in FIG. 27 with two or less, three or less, four or less, five or less, six or less, seven or less, eight or less, nine or less, or ten or less amino acid deletions, additions, substitutions, or combinations thereof.

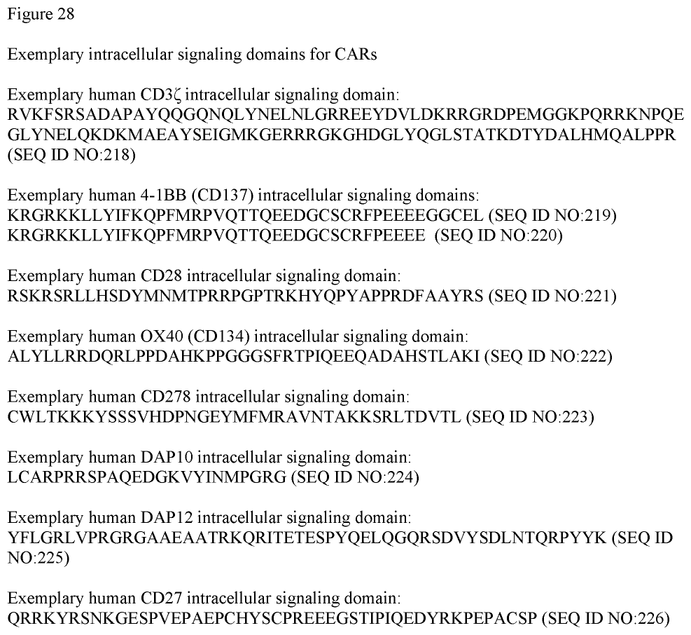

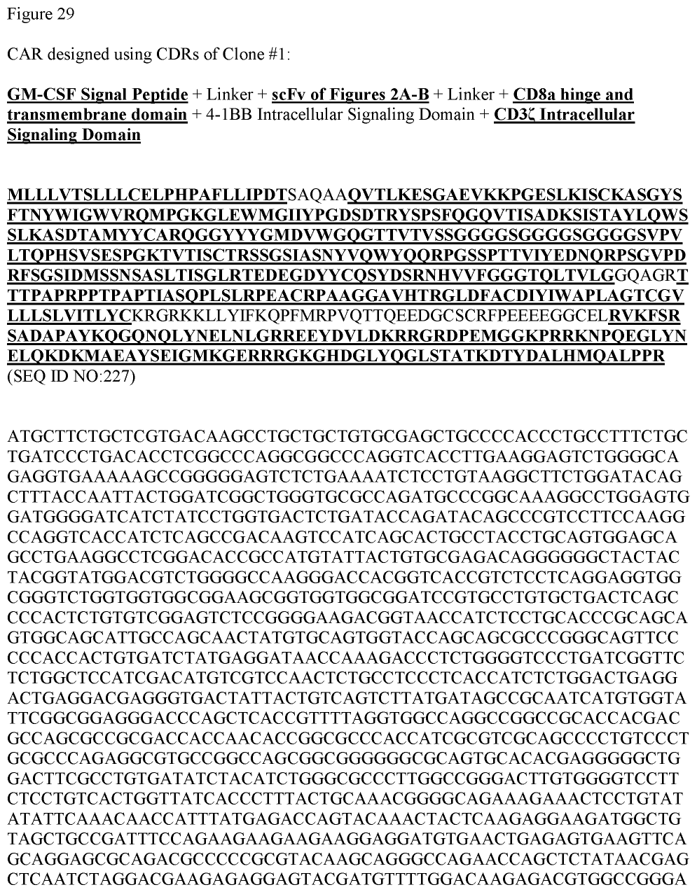

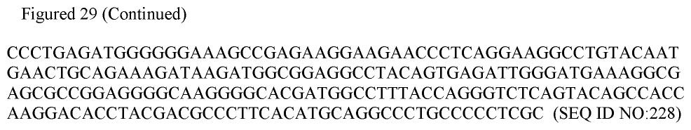

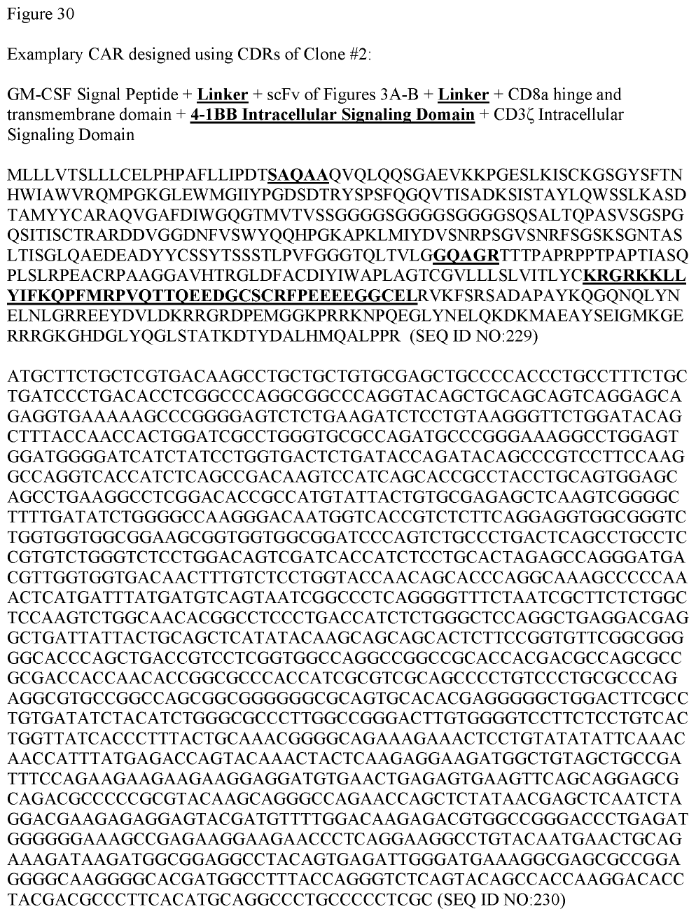

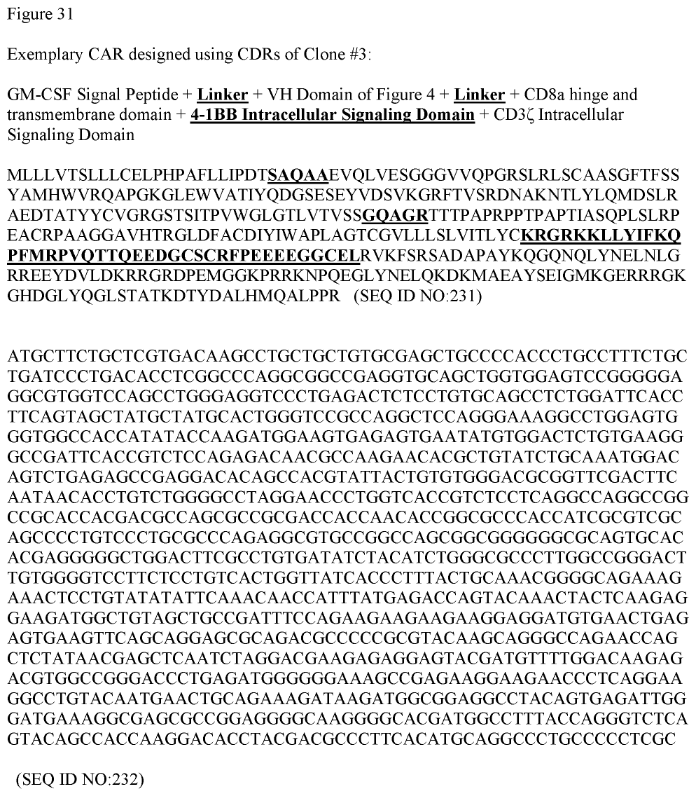

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. In some cases, a CAR provided herein can be designed to include at least one intracellular signaling domain that comprises, consists essentially of, or consists of one of the amino acid sequences set forth in FIG. 28. In some cases, a CAR provided herein can be designed to include at least one intracellular signaling domain that comprises, consists essentially of, or consists of one of the amino acid sequences set forth in FIG. 28 with one, two, three, four, five, six, seven, eight, nine, or ten amino acid deletions, additions, substitutions, or combinations thereof, provided that that intracellular signaling domain has at least some activity to activate intracellular signaling. In some cases, a CAR provided herein can be designed to include at least one intracellular signaling domain that comprises, consists essentially of, or consists of one of the amino acid sequences set forth in FIG. 28 with two or less, three or less, four or less, five or less, six or less, seven or less, eight or less, nine or less, or ten or less amino acid deletions, additions, substitutions, or combinations thereof, provided that that intracellular signaling domain has at least some activity to activate intracellular signaling.

In some cases, a CAR targeting a PSMA polypeptide can be designed to include an scFv having a heavy chain variable domain comprising SEQ ID NO:1, SEQ ID NO:2, and SEQ ID NO:3, followed by a linker such as a linker set forth in FIG. 22, followed by a light chain variable domain comprising SEQ ID NO:9, SEQ ID NO:10, and SEQ ID NO:11, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a heavy chain variable domain comprising SEQ ID NO:1, SEQ ID NO:2, and SEQ ID NO:3, followed by SEQ ID NO:168, followed by a light chain variable domain comprising SEQ ID NO:9, SEQ ID NO:10, and SEQ ID NO:11, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 29).

In some cases, a CAR targeting a PSMA polypeptide can be designed to include an scFv having a heavy chain variable domain comprising SEQ ID NO:8, followed by a linker such as a linker set forth in FIG. 22, followed by a light chain variable domain comprising SEQ ID NO:16, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a heavy chain variable domain comprising SEQ ID NO:8, followed by SEQ ID NO:168, followed by a light chain variable domain comprising SEQ ID NO:16, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 29).

In some cases, a CAR targeting a PSMA polypeptide can be designed to include an scFv having a light chain variable domain comprising SEQ ID NO:9, SEQ ID NO:10, and SEQ ID NO:11, followed by a linker such as a linker set forth in FIG. 22, followed by a heavy chain variable domain comprising SEQ ID NO:1, SEQ ID NO:2, and SEQ ID NO:3, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a light chain variable domain comprising SEQ ID NO:9, SEQ ID NO:10, and SEQ ID NO:11, followed by SEQ ID NO:168, followed by a heavy chain variable domain comprising SEQ ID NO:1, SEQ ID NO:2, and SEQ ID NO:3, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218.

In some cases, a CAR targeting a PSMA polypeptide can be designed to include an scFv having a light chain variable domain comprising SEQ ID NO:16, followed by a linker such as a linker set forth in FIG. 22, followed by a heavy chain variable domain comprising SEQ ID NO:8, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a light chain variable domain comprising SEQ ID NO:16, followed by SEQ ID NO:168, followed by a heavy chain variable domain comprising SEQ ID NO:8, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218.

In some cases, a CAR targeting a PSMA polypeptide can be designed to include an scFv having a heavy chain variable domain comprising SEQ ID NO:17, SEQ ID NO:18, and SEQ ID NO:19, followed by a linker such as a linker set forth in FIG. 22, followed by a light chain variable domain comprising SEQ ID NO:25, SEQ ID NO:26, and SEQ ID NO:27, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a heavy chain variable domain comprising SEQ ID NO:17, SEQ ID NO:18, and SEQ ID NO:19, followed by SEQ ID NO:169, followed by a light chain variable domain comprising SEQ ID NO:25, SEQ ID NO:26, and SEQ ID NO:27, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 30).

In some cases, a CAR targeting a PSMA polypeptide can be designed to include an scFv having a heavy chain variable domain comprising SEQ ID NO:24, followed by a linker such as a linker set forth in FIG. 22, followed by a light chain variable domain comprising SEQ ID NO:32, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a heavy chain variable domain comprising SEQ ID NO:24, followed by SEQ ID NO:169, followed by a light chain variable domain comprising SEQ ID NO:32, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 30).

In some cases, a CAR targeting a PSMA polypeptide can be designed to include an scFv having a light chain variable domain comprising SEQ ID NO:25, SEQ ID NO:26, and SEQ ID NO:27, followed by a linker such as a linker set forth in FIG. 22, followed by a heavy chain variable domain comprising SEQ ID NO:17, SEQ ID NO:18, and SEQ ID NO:19, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a light chain variable domain comprising SEQ ID NO:25, SEQ ID NO:26, and SEQ ID NO:27, followed by SEQ ID NO:169, followed by a heavy chain variable domain comprising SEQ ID NO:17, SEQ ID NO:18, and SEQ ID NO:19, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218.

In some cases, a CAR targeting a PSMA polypeptide can be designed to include an scFv having a light chain variable domain comprising SEQ ID NO:32, followed by a linker such as a linker set forth in FIG. 22, followed by a heavy chain variable domain comprising SEQ ID NO:24, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include an scFv having a light chain variable domain comprising SEQ ID NO:32, followed by SEQ ID NO:169, followed by a heavy chain variable domain comprising SEQ ID NO:24, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218.

In some cases, a CAR targeting a PSMA polypeptide can be designed to include a VH domain comprising SEQ ID NO:33, SEQ ID NO:34, and SEQ ID NO:35, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:33, SEQ ID NO:34, and SEQ ID NO:35, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 31).

In some cases, a CAR targeting a PSMA polypeptide can be designed to include a VH domain comprising SEQ ID NO:40, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:40, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 31).

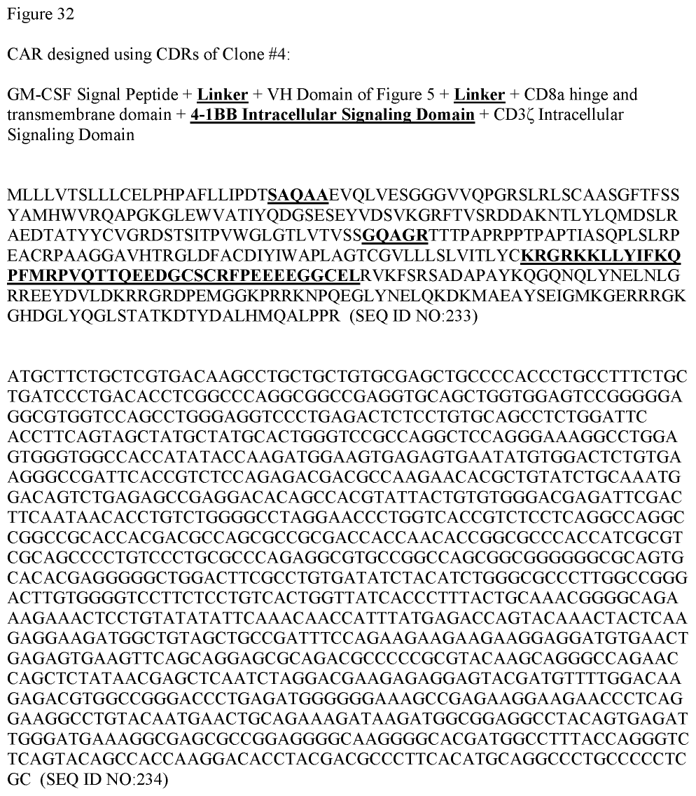

In some cases, a CAR targeting a PSMA polypeptide can be designed to include a VH domain comprising SEQ ID NO:41, SEQ ID NO:42, and SEQ ID NO:43, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:41, SEQ ID NO:42, and SEQ ID NO:43, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 32).

In some cases, a CAR targeting a PSMA polypeptide can be designed to include a VH domain comprising SEQ ID NO:48, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:48, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 32).

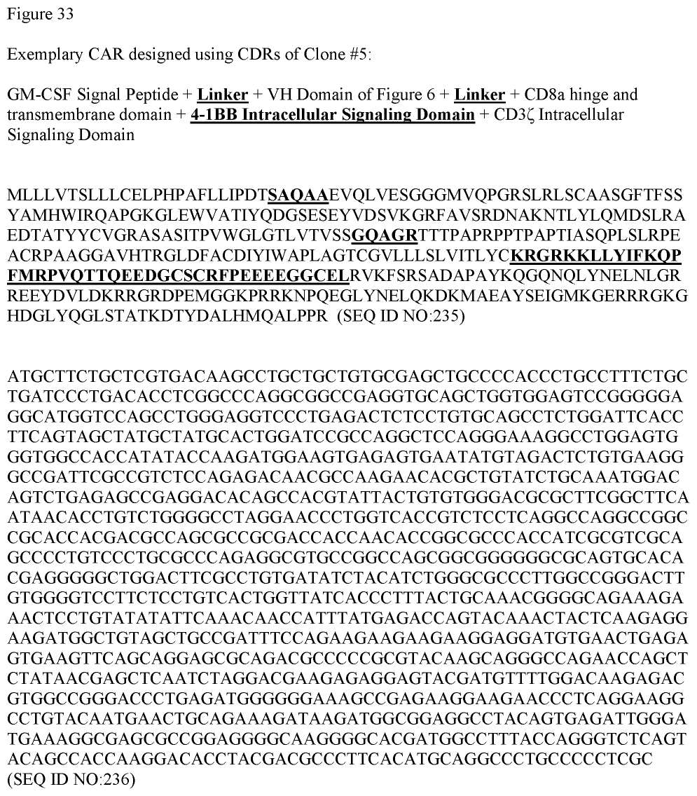

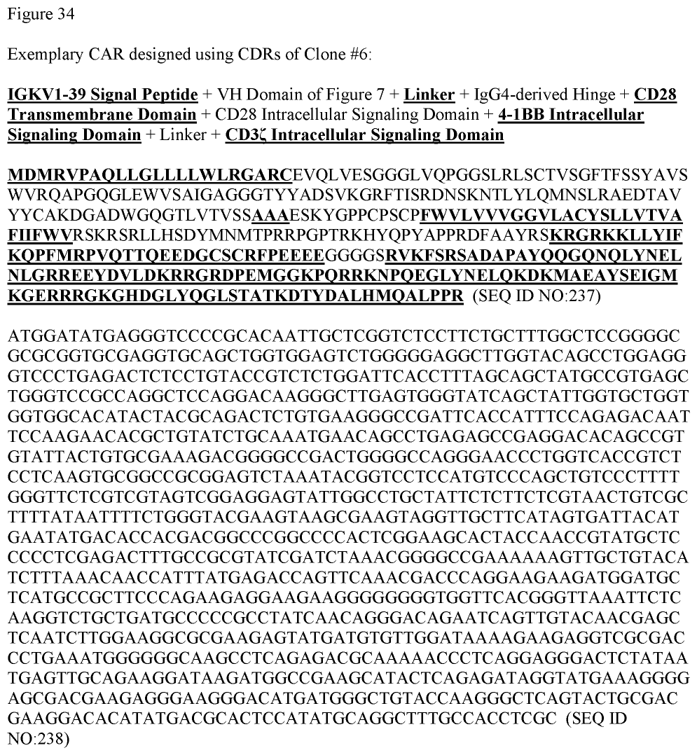

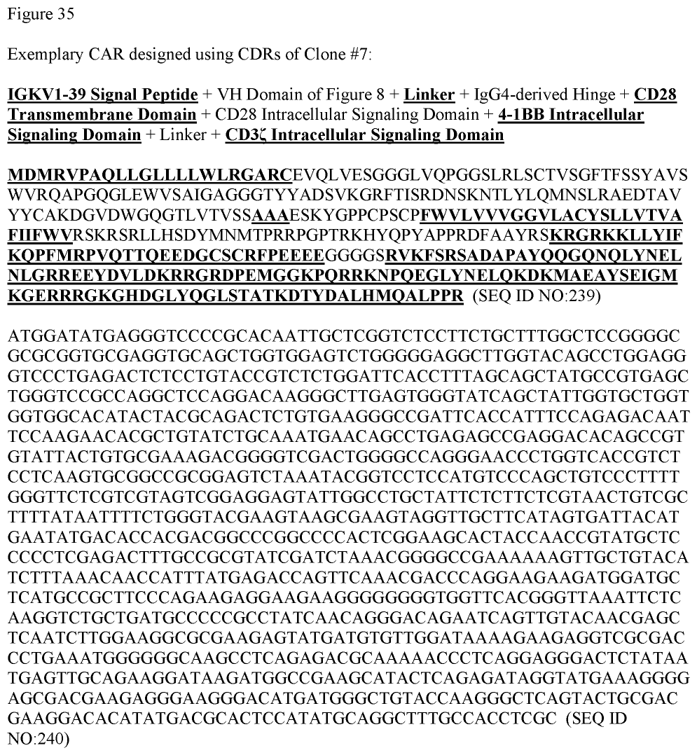

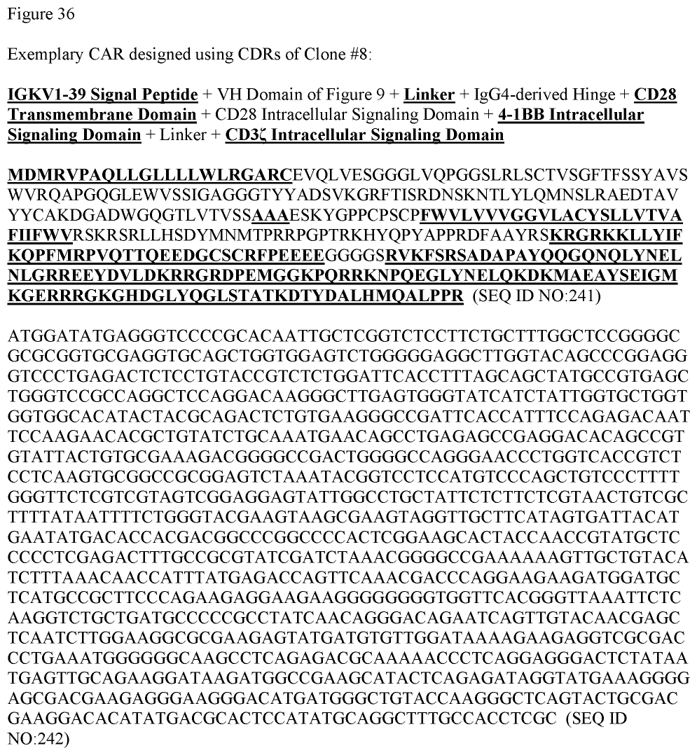

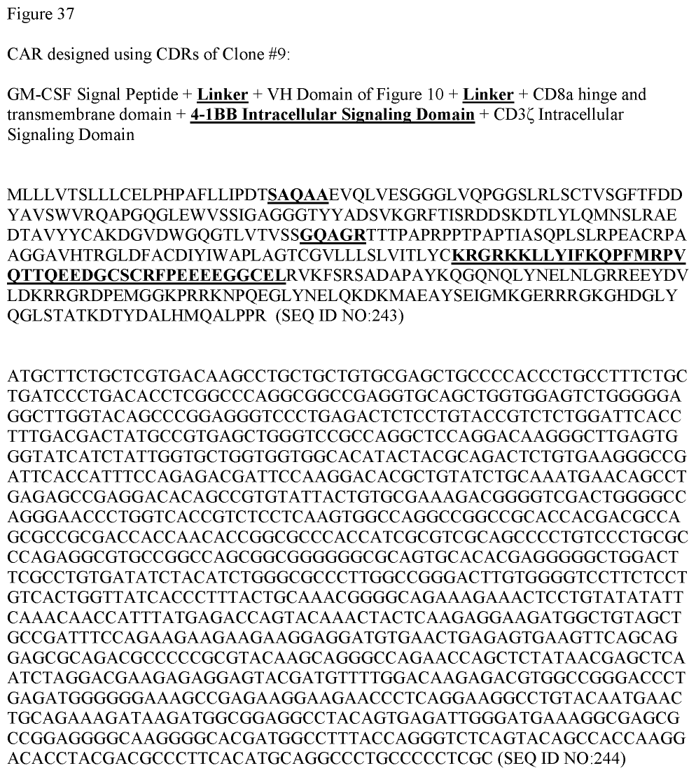

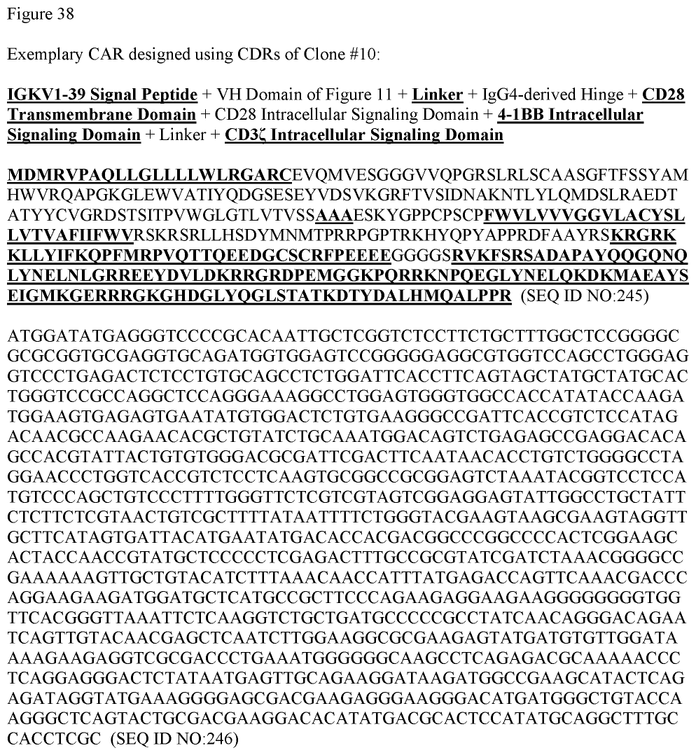

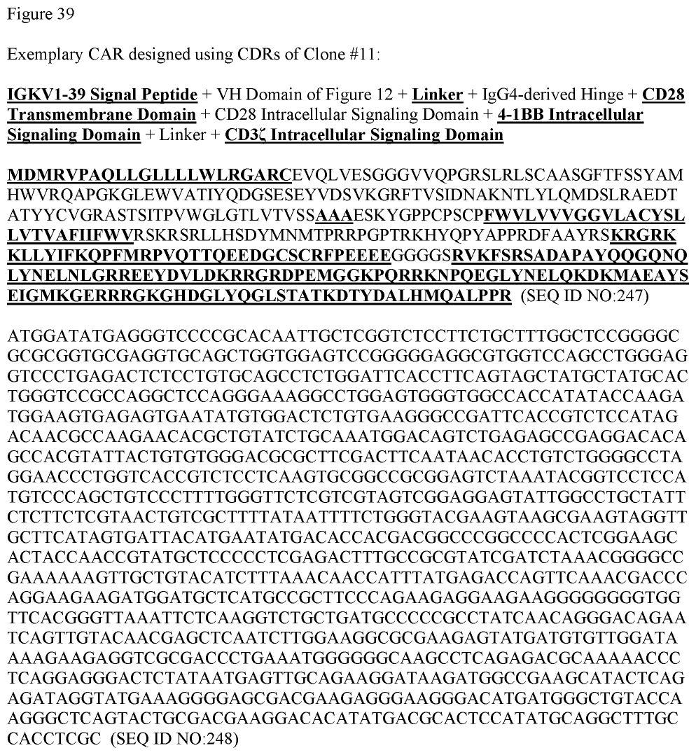

In some cases, a CAR targeting a PSMA polypeptide can be designed to include a VH domain comprising SEQ ID NO:49, SEQ ID NO:50, and SEQ ID NO:51, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:49, SEQ ID NO:50, and SEQ ID NO:51, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 33).

In some cases, a CAR targeting a PSMA polypeptide can be designed to include a VH domain comprising SEQ ID NO:56, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:56, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 33).

In some cases, a CAR targeting a PSMA polypeptide can be designed to include a VH domain comprising SEQ ID NO:57, SEQ ID NO:58, and SEQ ID NO:59, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:57, SEQ ID NO:58, and SEQ ID NO:59, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 34).

In some cases, a CAR targeting a PSMA polypeptide can be designed to include a VH domain comprising SEQ ID NO:64, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:64, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 34).

In some cases, a CAR targeting a PSMA polypeptide can be designed to include a VH domain comprising SEQ ID NO:65, SEQ ID NO:66, and SEQ ID NO:67, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:65, SEQ ID NO:66, and SEQ ID NO:67, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 35).

In some cases, a CAR targeting a PSMA polypeptide can be designed to include a VH domain comprising SEQ ID NO:72, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:72, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 35).

In some cases, a CAR targeting a PSMA polypeptide can be designed to include a VH domain comprising SEQ ID NO:73, SEQ ID NO:74, and SEQ ID NO:75, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:73, SEQ ID NO:74, and SEQ ID NO:75, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 36).

In some cases, a CAR targeting a PSMA polypeptide can be designed to include a VH domain comprising SEQ ID NO:80, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:80, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 36).

In some cases, a CAR targeting a PSMA polypeptide can be designed to include a VH domain comprising SEQ ID NO:81, SEQ ID NO:82, and SEQ ID NO:83, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:81, SEQ ID NO:82, and SEQ ID NO:83, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 37).

In some cases, a CAR targeting a PSMA polypeptide can be designed to include a VH domain comprising SEQ ID NO:88, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:88, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 37).

In some cases, a CAR targeting a PSMA polypeptide can be designed to include a VH domain comprising SEQ ID NO:89, SEQ ID NO:90, and SEQ ID NO:91, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:89, SEQ ID NO:90, and SEQ ID NO:91, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 38).

In some cases, a CAR targeting a PSMA polypeptide can be designed to include a VH domain comprising SEQ ID NO:96, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:96, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 38).

In some cases, a CAR targeting a PSMA polypeptide can be designed to include a VH domain comprising SEQ ID NO:97, SEQ ID NO:98, and SEQ ID NO:99, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:97, SEQ ID NO:98, and SEQ ID NO:99, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 39).

In some cases, a CAR targeting a PSMA polypeptide can be designed to include a VH domain comprising SEQ ID NO:104, followed by a hinge such as a hinge/linker set forth in FIG. 22 or FIG. 26 (e.g., an IgG4-derived hinge, a CD8a hinge, or a linker plus IgG4-derived hinge), followed by a transmembrane domain such as a transmembrane domain set forth in FIG. 27 (e.g., a human CD28 transmembrane domain or a CD8a transmembrane domain), followed by one or more intracellular signaling domains such as one or more intracellular signaling domain set forth in FIG. 28 (e.g., a human 4-1BB intracellular signaling domain followed by a human CD3ζ intracellular signaling domain). For example, a CAR targeting a PSMA polypeptide can be designed to include a VH domain comprising SEQ ID NO:104, followed by SEQ ID NO:194, followed by SEQ ID NO:205, followed by SEQ ID NO:216, followed by SEQ ID NO:221, followed by SEQ ID NO:220, followed by SEQ ID NO:189, followed by SEQ ID NO:218 (see, e.g., FIG. 39).

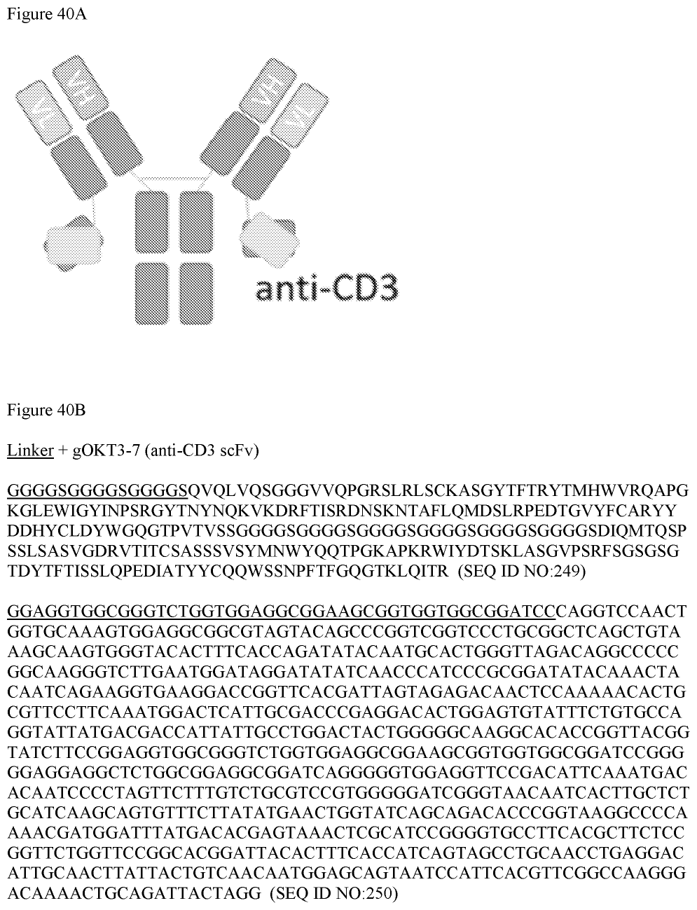

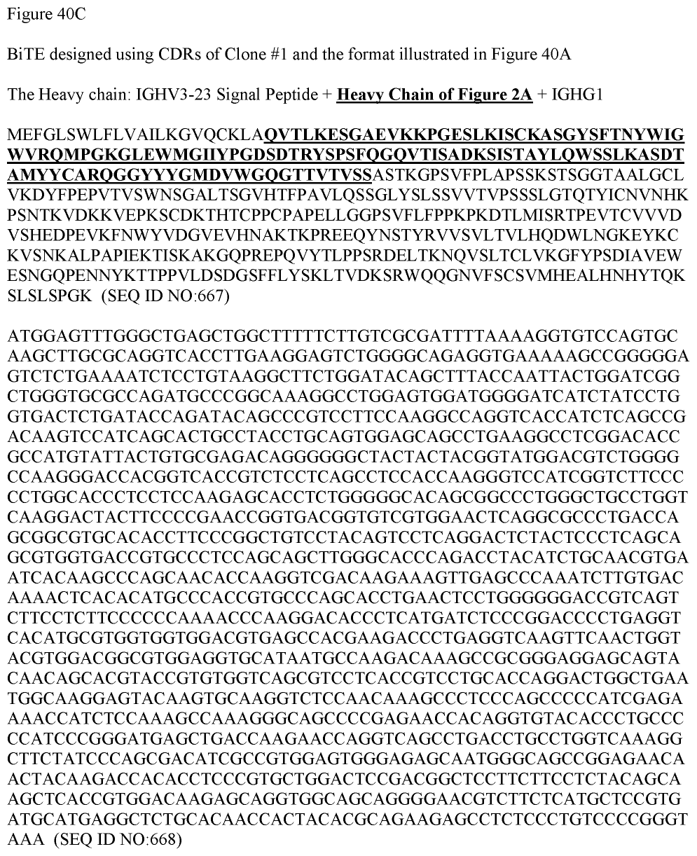

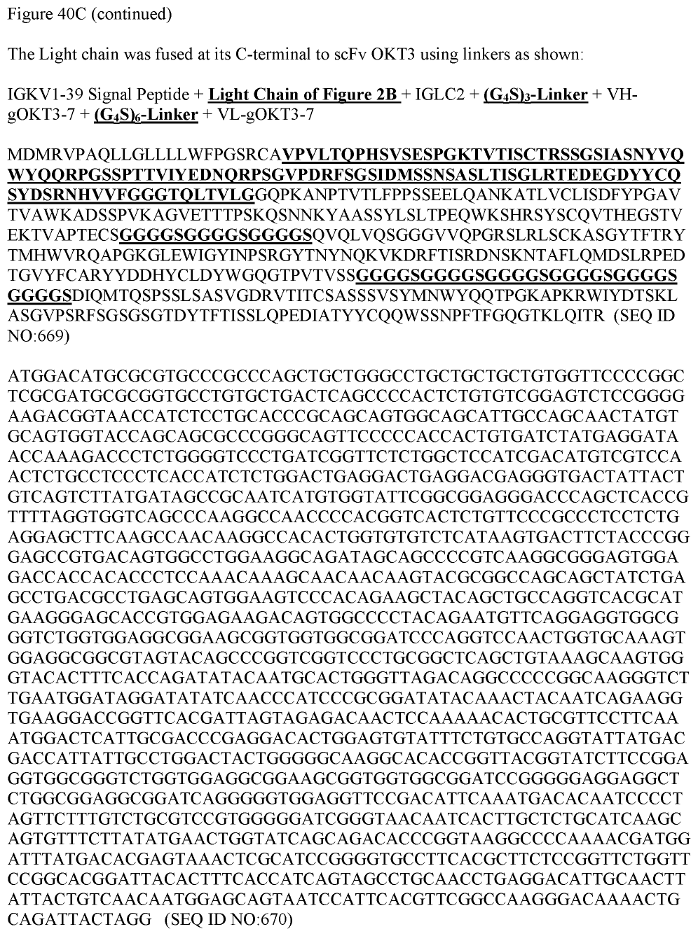

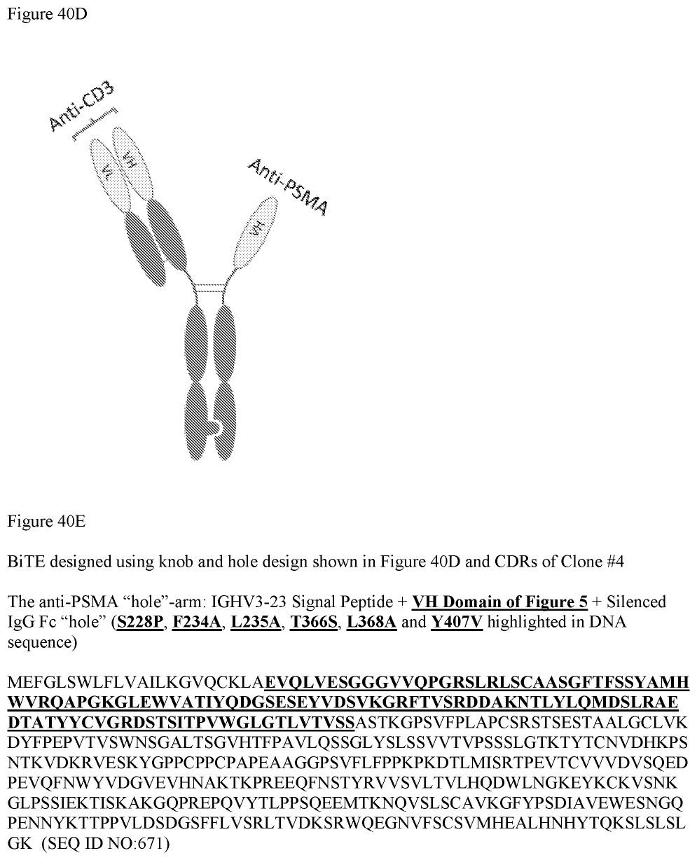

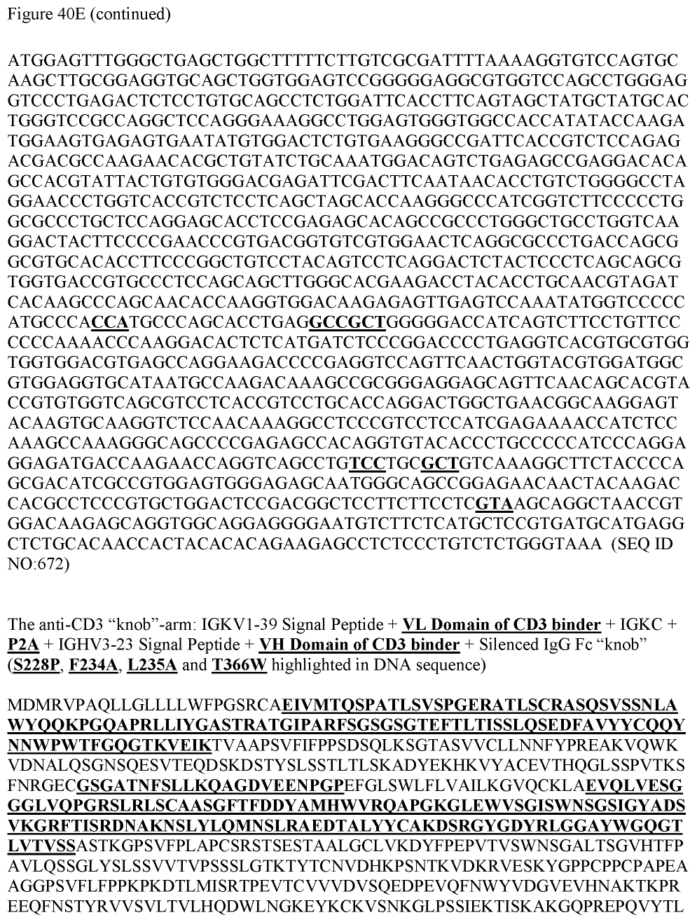

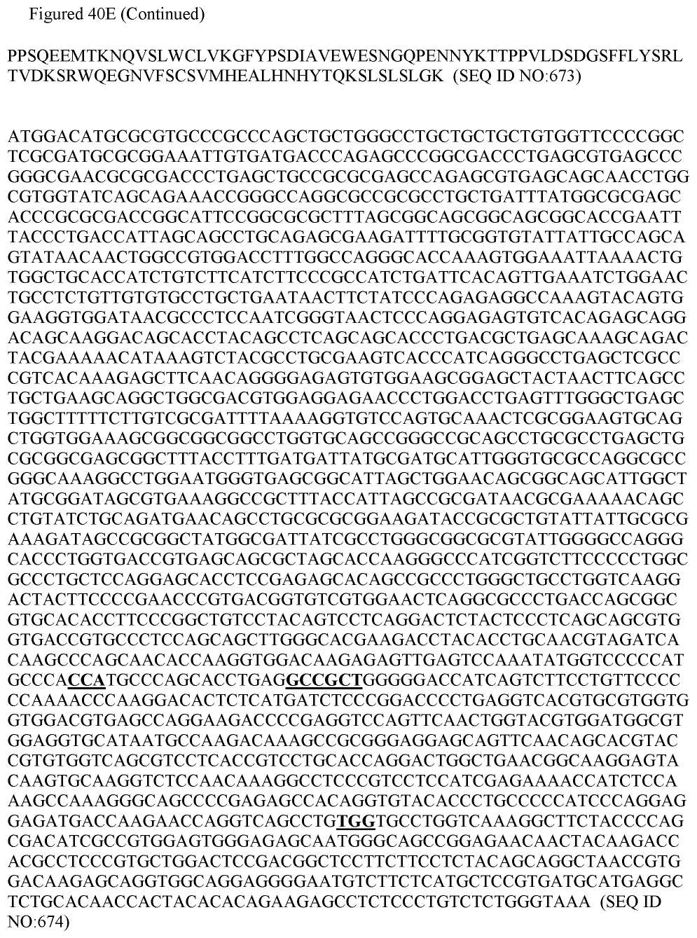

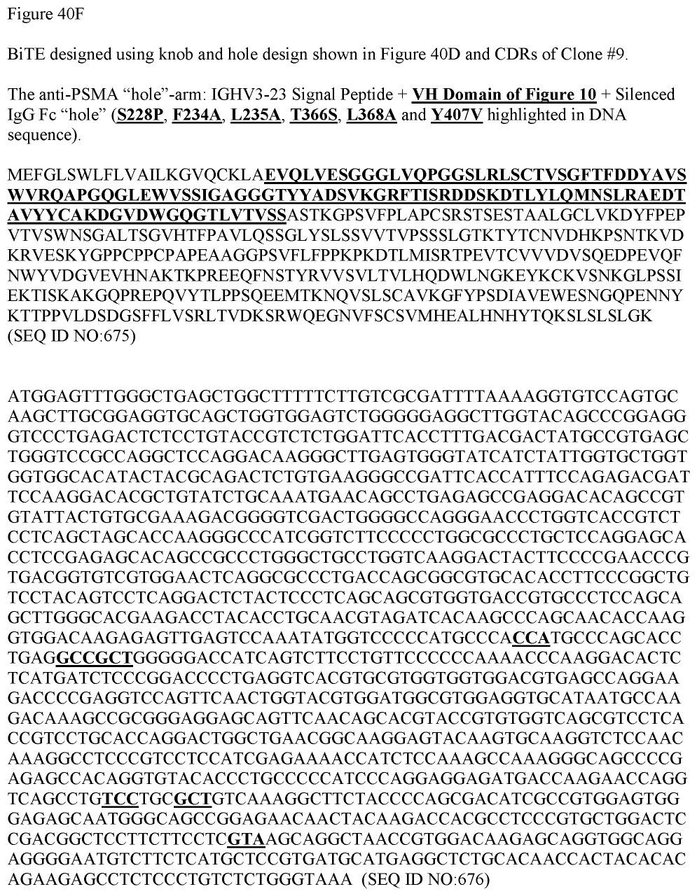

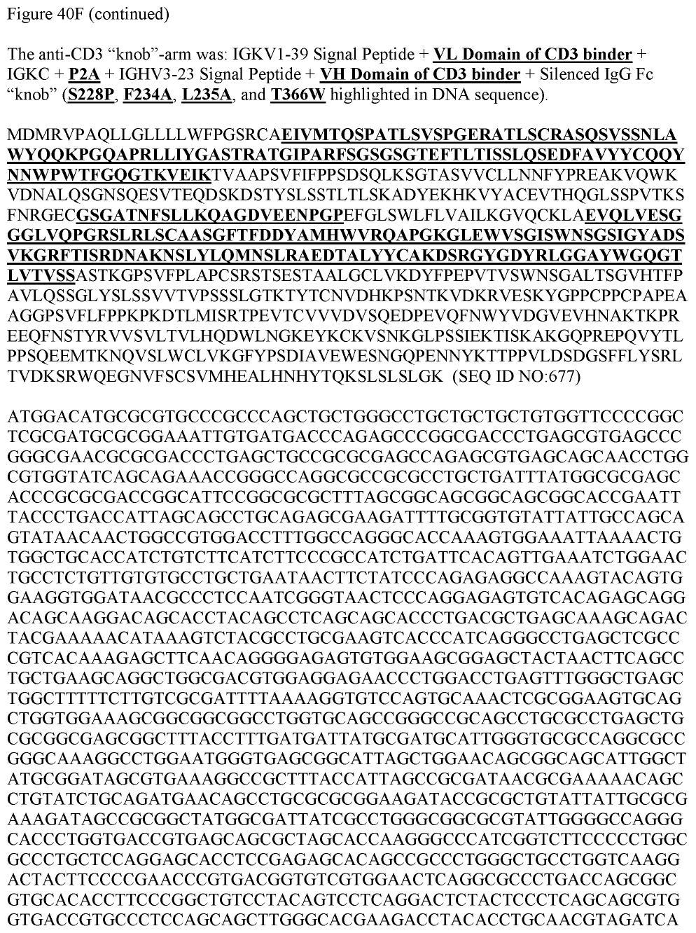

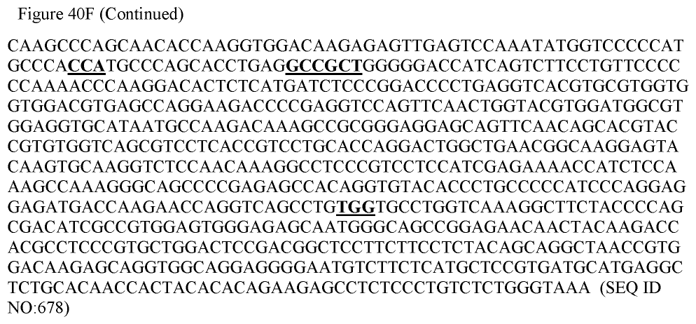

The term “cell engager” as used herein refers to a polypeptide that includes two or more antigen binding domains (e.g., two, three, or four antigen binding domains) and has the ability to link two cells together. Examples of cell engagers include, without limitation, BiTEs, BiKEs, and TriKEs. In general, a cell engager provided herein can be designed to include at least one antigen binding domain having the ability to bind to a PSMA polypeptide (e.g., a human PSMA polypeptide) and at least one antigen binding domain having the ability to bind to an antigen expressed on the surface of a cell (e.g., a T cell or an NK cell). In some cases, a cell engager described herein can link a PSMA⁺ cell (e.g., a PSMA⁺ cancer cell) to another cell (e.g., a T cell or an NK cell) via the two or more antigen binding domains of the cell engager. An example of a cell engager structure of cell engagers provided herein includes, without limitation, the structure set forth in FIG. 40A. In some cases, the anti-CD3 scFv depicted in FIG. 40A can be replace with a different antigen binding domain having the ability to bind to an antigen expressed on the surface of a cell (e.g., a T cell or an NK cell).

When a cell engager includes an antigen binding domain having the ability to bind to a PSMA polypeptide (e.g., a human PSMA polypeptide) and two or more other antigen binding domains (e.g., two, three, or four other antigen binding domains), each of those other antigen binding domains can bind to different antigens expressed on the surface of different cell types or can bind to different antigens expressed on the surface of the same cell type. For example, a TriKE can be designed to have a first antigen binding domain having the ability to bind to a PSMA polypeptide (e.g., a human PSMA polypeptide), a second antigen binding domain having the ability to bind to a first antigen expressed on the surface of an NK cell (e.g., a CD16 polypeptide such as a CD16a polypeptide), and a third antigen binding domain having the ability to bind to a second antigen expressed on the surface of an NK cell (e.g., an NKG2A polypeptide).

As described herein, at least one antigen binding domain of a cell engager provided herein can be designed to bind to a PSMA polypeptide (e.g., a human PSMA polypeptide). For example, a cell engager provided herein can be designed to include the components of an antibody, antigen binding fragment, and/or antibody domain described herein (e.g., a combination of CDRs) as an antigen binding domain provided that that antigen binding domain has the ability to bind to a PSMA polypeptide (e.g., a human PSMA polypeptide). In some examples, a cell engager provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 9-11 or SEQ ID NOs:17-19 and 25-27).

In some examples, a cell engager provided herein can be designed to include an antigen binding domain that includes a single set of three CDRs (e.g., a CDR1, CDR2, and CDR3) of an antibody domain (e.g., a VH domain) provided herein (e.g., SEQ ID NOs:33-35; SEQ ID NOs:41-43; SEQ ID NOs:49-51; SEQ ID NOs:57-59; SEQ ID NOs:65-67; SEQ ID NOs:73-75; SEQ ID NOs:81-83; SEQ ID NOs:89-91; or SEQ ID NOs:97-99).

In some cases, an antigen binding domain of a cell engager targeting a PSMA polypeptide can be designed to include a VH domain described herein or a scFv/Fab antibody described herein. In some cases, an antigen binding domain of a CAR described herein that has the ability to bind to a PSMA polypeptide (e.g., a human PSMA polypeptide) can be used as an antigen binding domain of a cell engager that targets PSMA⁺ cells.

As described herein, a cell engager can be designed to include at least one antigen binding domain having the ability to bind to a PSMA polypeptide (e.g., a human PSMA polypeptide) and at least one other antigen binding domain. That at least one other antigen binding domain can have the ability to bind to any appropriate antigen expressed on the surface of a cell. For example, when designing a cell engager such as a BiTE to link a PSMA⁺ cell and a T cell, the cell engager can include an antigen binding domain having the ability to bind to a PSMA polypeptide (e.g., a human PSMA polypeptide) and an antigen binding domain having the ability to bind to a polypeptide expressed on the surface of a T cell. Examples example of polypeptides expressed on the surface of a T cell that can be targeted by an antigen binding domain of a cell engager provided herein include, without limitation, CD3 polypeptides. Examples of antigen binding domains having the ability to bind to a polypeptide expressed on the surface of a T cell that can be used to make a cell engager provided herein (e.g., a BiTE) include, without limitation, anti-CD3 scFvs and anti-CD3 VH domains. Additional examples of amino acid sequences that can be used as antigen binding domains having the ability to bind to a polypeptide expressed on the surface of a T cell (e.g., CD3) are described in U.S. Pat. No. 6,750,325 (see, e.g., the sequence listing of U.S. Pat. No. 6,750,325).

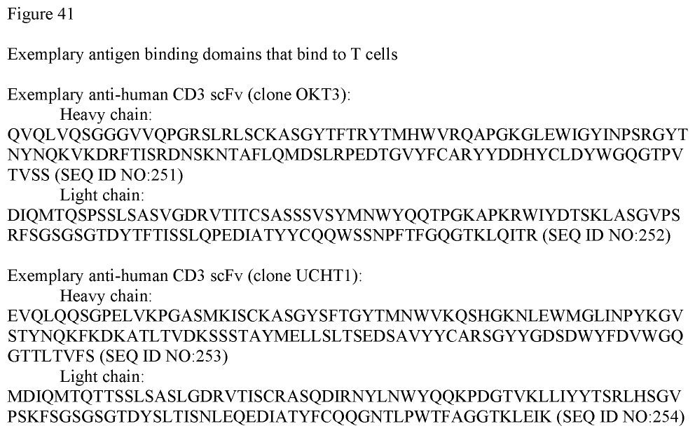

In some cases, a cell engager provided herein can be designed to include an antigen binding domain that comprises, consists essentially of, or consists of one of the amino acid sequences set forth in FIG. 41. In some cases, a cell engager provided herein can be designed to include an antigen binding domain that comprises, consists essentially of, or consists of one of the amino acid sequences set forth in FIG. 41 with one, two, three, four, five, six, seven, eight, nine, or ten amino acid deletions, additions, substitutions, or combinations thereof, provided that the antigen binding domain has the ability to bind to a polypeptide expressed on the surface of a T cell. In some cases, a cell engager provided herein can be designed to include an antigen binding domain that comprises, consists essentially of, or consists of one of the amino acid sequences set forth in FIG. 41 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of a T cell.

When designing a cell engager such as a BiKE or a TriKE to link a PSMA⁺ cell and an NK cell, the cell engager can include an antigen binding domain having the ability to bind to a PSMA polypeptide (e.g., a human PSMA polypeptide) and one or more (e.g., one, two, or three) antigen binding domains having the ability to bind to a polypeptide expressed on the surface of an NK cell. Examples of polypeptides expressed on the surface of an NK cell that can be targeted by an antigen binding domain of a cell engager provided herein include, without limitation, CD16 polypeptides (e.g., CD16a polypeptides), NKG2A polypeptides, NKG2D polypeptides, NKp30 polypeptides, NKp44 polypeptides, and NKp46 polypeptides. Examples of antigen binding domains having the ability to bind to a polypeptide expressed on the surface of an NK cell that can be used to make a cell engager provided herein (e.g., a BiKE or TriKE) include, without limitation, anti-CD16a scFvs, anti-NKG2A scFvs, anti-NKG2D scFvs, anti-NKp30 scFvs (see, e.g., BioLegend Catalog #325207), anti-NKp44 scFvs, anti-NKp46 scFvs, anti-CD16a VH domains, anti-NKG2A VH domains, anti-NKG2D VH domains, anti-NKp30 VH domains, anti-NKp44 VH domains, and anti-NKp46 VH domains. Additional examples of amino acid sequences that can be used as antigen binding domains having the ability to bind to a polypeptide expressed on the surface of an NK cell (e.g., CD16, NKG2A, NKG2D, or NKp46) are described in McCall et al. (Mol. Immunol., 36(7):433-445 (1999); see, e.g., anti-CD16 scFv sequences); International Patent Application Publication No. PCT/US2017/048721 (see, e.g., the CDRs and sequence listing for anti-CD16a binding domains); U.S. Patent Application Publication No. 2011/0052606 (see, e.g., the CDRs and the sequence listing for anti-NKG2A antibodies such as Z199); U.S. Patent Application Publication No. 2011/0150870 (see, e.g., the CDRs and sequence listing for anti-NKG2D antibodies); U.S. Patent Application Publication No. 2018/0369373 (see, e.g., the CDRs and sequence listing for anti-NKp46 antibodies); and U.S. Patent Application Publication No. 2017/0368169 (see, e.g., the CDRs and sequence listing for anti-NKp46 antibodies).

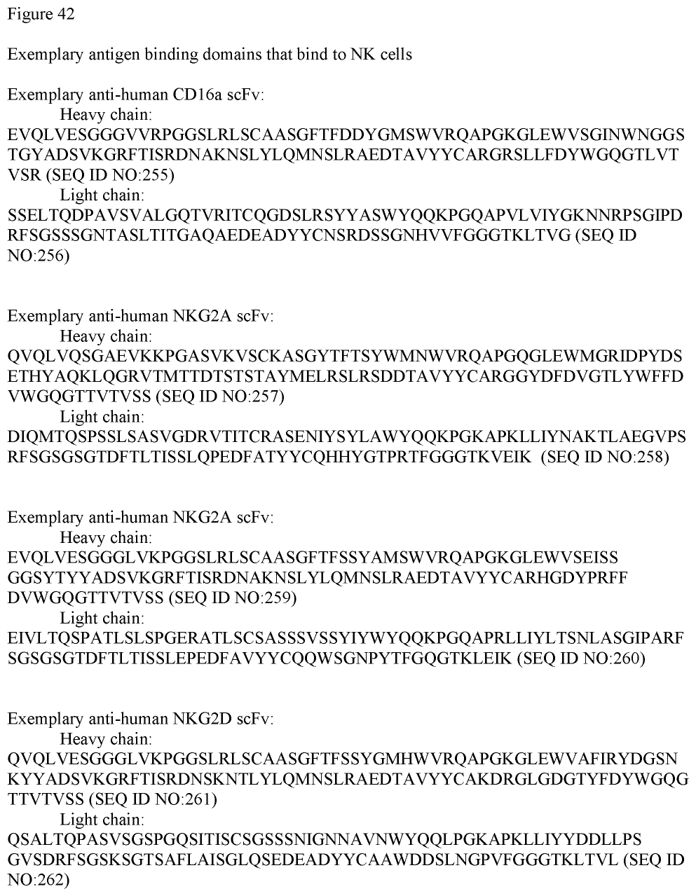

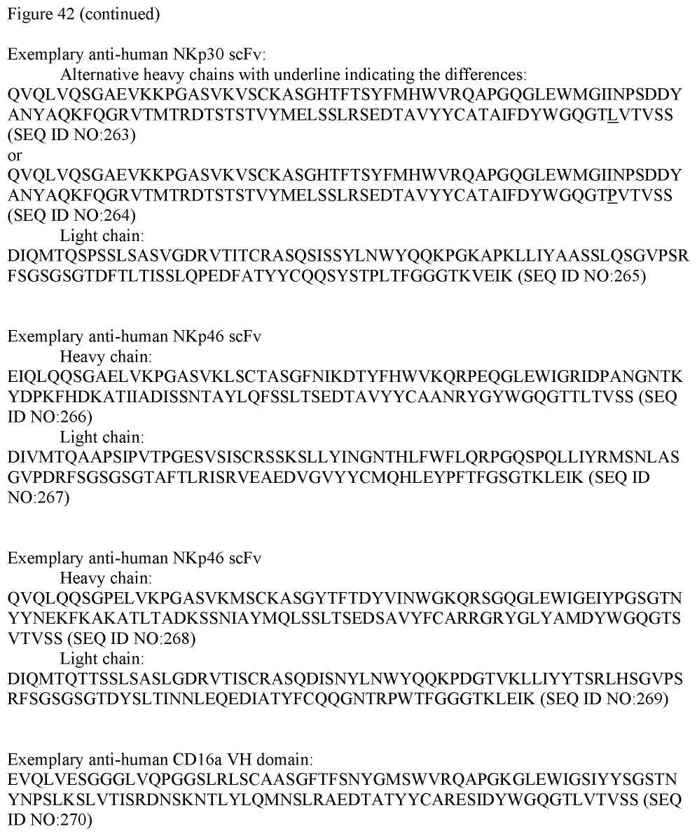

In some cases, a cell engager provided herein can be designed to include an antigen binding domain (e.g., a scFv or VH) that comprises, consists essentially of, or consists of one or more of the amino acid sequences set forth in FIG. 42. In some cases, a cell engager provided herein can be designed to include an antigen binding domain (e.g., a scFv or VH) that comprises, consists essentially of, or consists of one of the amino acid sequences set forth in FIG. 42 with one, two, three, four, five, six, seven, eight, nine, or ten amino acid deletions, additions, substitutions, or combinations thereof, provided that the antigen binding domain has the ability to bind to a polypeptide expressed on the surface of an NK cell. In some cases, a cell engager provided herein can be designed to include an antigen binding domain (e.g., a scFv or VH) that comprises, consists essentially of, or consists of one of the amino acid sequences set forth in FIG. 42 with two or less, three or less, four or less, five or less, six or less, seven or less, eight or less, nine or less, or ten or less amino acid deletions, additions, substitutions, or combinations thereof, provided that the antigen binding domain has the ability to bind to a polypeptide expressed on the surface of an NK cell.

In some cases, a cell engager provided herein can be designed to include a linker located between each antigen binding domain. Any appropriate linker can be used to design a cell engager provided herein. Examples of linkers that can be used to make a cell engager described herein include, without limitation, the linker sequences set forth in FIG. 22. A cell engager provided herein can be designed to include a linker of any appropriate length. For example, a cell engager provided herein can be designed to include a linker that is from about 3 to about 100 (e.g., from about 3 to about 90, from about 3 to about 80, from about 3 to about 70, from about 3 to about 60, from about 3 to about 50, from about 3 to about 40, from about 3 to about 30, from about 3 to about 20, from about 3 to about 15, from about 5 to about 100, from about 10 to about 100, from about 20 to about 100, from about 30 to about 100, from about 40 to about 100, from about 50 to about 100, from about 60 to about 100, from about 70 to about 100, from about 10 to about 50, from about 10 to about 40, from about 10 to about 30, from about 10 to about 20, or from about 12 to about 17) amino acid residues in length. In some cases, a cell engager provided herein (e.g., a BiTE) can be designed to include a GGGGSGGGGSGGGGS (SEQ ID NO:170) linker. In some cases, a hinge of a CAR described herein can be used as a linker to make a cell engager described herein. For example, any one of the sequences set forth in FIG. 26 can be used as a linker of a cell engager described herein.

In some cases, a cell engager provided herein can be designed to include a linker that comprises, consists essentially of, or consists of one of the amino acid sequences set forth in FIG. 22 or FIG. 26. In some cases, a cell engager provided herein can be designed to include a linker that comprises, consists essentially of, or consists of one of the amino acid sequences set forth in FIG. 22 or FIG. 26 with one, two, three, four, five, six, seven, eight, nine, or ten amino acid deletions, additions, substitutions, or combinations thereof. In some cases, a cell engager provided herein can be designed to include a linker that comprises, consists essentially of, or consists of one of the amino acid sequences set forth in FIG. 22 or FIG. 26 with two or less, three or less, four or less, five or less, six or less, seven or less, eight or less, nine or less, or ten or less amino acid deletions, additions, substitutions, or combinations thereof.

In some cases, a cell engager (e.g., a BiTE) targeting a PSMA polypeptide can be designed to include an scFv having a heavy chain variable domain comprising SEQ ID NO:1, SEQ ID NO:2, and SEQ ID NO:3, followed by a linker such as a linker set forth in FIG. 22, followed by a light chain variable domain comprising SEQ ID NO:9, SEQ ID NO:10, and SEQ ID NO:11, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an scFv having a light chain variable domain comprising SEQ ID NO:9, SEQ ID NO:10, and SEQ ID NO:11, followed by a linker such as a linker set forth in FIG. 22, followed by a heavy chain variable domain comprising SEQ ID NO:1, SEQ ID NO:2, and SEQ ID NO:3, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an scFv having a heavy chain variable domain comprising SEQ ID NO:8, followed by a linker such as a linker set forth in FIG. 22, followed by a light chain variable domain comprising SEQ ID NO:16, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an scFv having a light chain variable domain comprising SEQ ID NO:16, followed by a linker such as a linker set forth in FIG. 22, followed by a heavy chain variable domain comprising SEQ ID NO:8, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

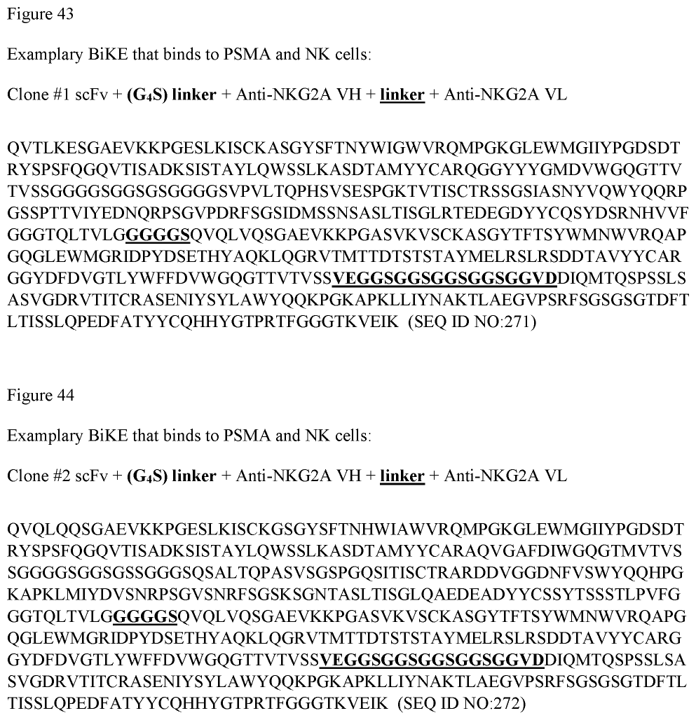

In some cases, a cell engager (e.g., a BiKE or a TriKE) targeting a PSMA polypeptide can be designed to include an scFv having a heavy chain variable domain comprising SEQ ID NO:1, SEQ ID NO:2, and SEQ ID NO:3, followed by a linker such as a linker set forth in FIG. 22, followed by a light chain variable domain comprising SEQ ID NO:9, SEQ ID NO:10, and SEQ ID NO:11, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 43).

In some cases, a cell engager (e.g., a BiKE or a TriKE) targeting a PSMA polypeptide can be designed to include an scFv having a light chain variable domain comprising SEQ ID NO:9, SEQ ID NO:10, and SEQ ID NO:11, followed by a linker such as a linker set forth in FIG. 22, followed by a heavy chain variable domain comprising SEQ ID NO:1, SEQ ID NO:2, and SEQ ID NO:3, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include an scFv having a heavy chain variable domain comprising SEQ ID NO:8, followed by a linker such as a linker set forth in FIG. 22, followed by a light chain variable domain comprising SEQ ID NO:16, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 43).

In some cases, a cell engager (e.g., a BiKE or a TriKE) targeting a PSMA polypeptide can be designed to include an scFv having a light chain variable domain comprising SEQ ID NO:16, followed by a linker such as a linker set forth in FIG. 22, followed by a heavy chain variable domain comprising SEQ ID NO:8, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include an scFv having a heavy chain variable domain comprising SEQ ID NO:17, SEQ ID NO:18, and SEQ ID NO:19, followed by a linker such as a linker set forth in FIG. 22, followed by a light chain variable domain comprising SEQ ID NO:25, SEQ ID NO:26, and SEQ ID NO:27, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an scFv having a light chain variable domain comprising SEQ ID NO:25, SEQ ID NO:26, and SEQ ID NO:27, followed by a linker such as a linker set forth in FIG. 22, followed by a heavy chain variable domain comprising SEQ ID NO:17, SEQ ID NO:18, and SEQ ID NO:19, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an scFv having a heavy chain variable domain comprising SEQ ID NO:24, followed by a linker such as a linker set forth in FIG. 22, followed by a light chain variable domain comprising SEQ ID NO:32, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an scFv having a light chain variable domain comprising SEQ ID NO:32, followed by a linker such as a linker set forth in FIG. 22, followed by a heavy chain variable domain comprising SEQ ID NO:24, followed by a linker such as a hinge/linker set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include an scFv having a heavy chain variable domain comprising SEQ ID NO:17, SEQ ID NO:18, and SEQ ID NO:19, followed by a linker such as a linker set forth in FIG. 22, followed by a light chain variable domain comprising SEQ ID NO:25, SEQ ID NO:26, and SEQ ID NO:27, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 44).

In some cases, a cell engager (e.g., a BiKE or a TriKE) targeting a PSMA polypeptide can be designed to include an scFv having a light chain variable domain comprising SEQ ID NO:25, SEQ ID NO:26, and SEQ ID NO:27, followed by a linker such as a linker set forth in FIG. 22, followed by a heavy chain variable domain comprising SEQ ID NO:17, SEQ ID NO:18, and SEQ ID NO:19, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include an scFv having a heavy chain variable domain comprising SEQ ID NO:24, followed by a linker such as a linker set forth in FIG. 22, followed by a light chain variable domain comprising SEQ ID NO:32, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE) (see, e.g., FIG. 44).

In some cases, a cell engager (e.g., a BiKE or a TriKE) targeting a PSMA polypeptide can be designed to include an scFv having a light chain variable domain comprising SEQ ID NO:32, followed by a linker such as a linker set forth in FIG. 22, followed by a heavy chain variable domain comprising SEQ ID NO:24, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:33, SEQ ID NO:34, and SEQ ID NO:35, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:40, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:33, SEQ ID NO:34, and SEQ ID NO:35, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:40, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:41, SEQ ID NO:42, and SEQ ID NO:43, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:48, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:41, SEQ ID NO:42, and SEQ ID NO:43, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:48, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:49, SEQ ID NO:50, and SEQ ID NO:51, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:56, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:49, SEQ ID NO:50, and SEQ ID NO:51, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:56, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:57, SEQ ID NO:58, and SEQ ID NO:59, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:64, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:57, SEQ ID NO:58, and SEQ ID NO:59, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:64, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:65, SEQ ID NO:66, and SEQ ID NO:67, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:72, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:65, SEQ ID NO:66, and SEQ ID NO:67, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:72, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE). In some cases, a cell engager (e.g., a BiTE) targeting a PSMA polypeptide can be designed to include a VH domain comprising SEQ ID NO:73, SEQ ID NO:74, and SEQ ID NO:75, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:80, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:73, SEQ ID NO:74, and SEQ ID NO:75, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:80, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:81, SEQ ID NO:82, and SEQ ID NO:83, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:88, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:81, SEQ ID NO:82, and SEQ ID NO:83, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:88, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:89, SEQ ID NO:90, and SEQ ID NO:91, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:96, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:89, SEQ ID NO:90, and SEQ ID NO:91, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:96, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include a VH domain comprising SEQ ID NO:97, SEQ ID NO:98, and SEQ ID NO:99, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include a VH domain comprising SEQ ID NO:104, followed by a linker such as a linker/hinge set forth in FIG. 22 or FIG. 26 (e.g., SEQ ID NO:170), followed by an antigen binding domain having the ability to bind to a polypeptide expressed on the surface of a T cell (e.g., an anti-human CD3 scFv).

In some cases, a cell engager (e.g., a BiKE or a TriKE) targeting a PSMA polypeptide can be designed to include a VH domain comprising SEQ ID NO:97, SEQ ID NO:98, and SEQ ID NO:99, followed by a linker such as a linker/hinge set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiKE or a TriKE) targeting a PSMA polypeptide can be designed to include a VH domain comprising SEQ ID NO:104, followed by a linker such as a hinge/linker set forth in FIG. 22 or FIG. 26 (e.g., SEQ ID NO:170), followed by one or more antigen binding domains having the ability to bind to a polypeptide expressed on the surface of an NK cell (e.g., an anti-human CD16a scFv for a BiKE or an anti-human CD16a scFv and an anti-human NKG2A scFv for a TriKE).

In some cases, a cell engager (e.g., a BiTE) targeting a PSMA polypeptide can be designed to include an IgG (e.g., IgG1) configuration having (a) a heavy chain comprising, consisting essentially of, or consisting of a heavy chain variable domain comprising SEQ ID NO:1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an IgG (e.g., IgG1) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting a PSMA polypeptide can be designed to include an IgG (e.g., IgG1) configuration having (a) a heavy chain comprising, consisting essentially of, or consisting of a heavy chain variable domain comprising SEQ ID NO:1, SEQ ID NO:2, and SEQ ID NO:3, an Ig hinge, and constant domains (e.g., CH1, CH2, and CH3 domains) and (b) a light chain comprising, consisting essentially of, or consisting of a light chain variable domain comprising SEQ ID NO:9, SEQ ID NO:10, and SEQ ID NO:11, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting a PSMA polypeptide can be designed to include an IgG (e.g., IgG1) configuration having (a) a heavy chain comprising, consisting essentially of, or consisting of a heavy chain variable domain comprising SEQ ID NO:8, an Ig hinge, and constant domains (e.g., CH1, CH2, and CH3 domains) and (b) a light chain comprising, consisting essentially of, or consisting of a light chain variable domain comprising SEQ ID NO:16, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiTE) targeting a PSMA polypeptide can be designed to include an IgG (e.g., IgG1) configuration having (a) a heavy chain comprising, consisting essentially of, or consisting of a heavy chain variable domain comprising SEQ ID NO:17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiTE) targeting a PSMA polypeptide can be designed to include an IgG (e.g., IgG1) configuration having (a) a heavy chain comprising, consisting essentially of, or consisting of a heavy chain variable domain comprising SEQ ID NO:24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of a T cell (e.g., an anti-human CD3 scFv).

In some cases, a cell engager (e.g., a BiKE) targeting a PSMA polypeptide can be designed to include an IgG (e.g., IgG1) configuration having (a) a heavy chain comprising, consisting essentially of, or consisting of a heavy chain variable domain comprising SEQ ID NO:17, SEQ ID NO:18, and SEQ ID NO:19, an Ig hinge, and constant domains (e.g., CH1, CH2, and CH3 domains) and (b) a light chain comprising, consisting essentially of, or consisting of a light chain variable domain comprising SEQ ID NO:25, SEQ ID NO:26, and SEQ ID NO:27, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In some cases, a cell engager (e.g., a BiKE) targeting a PSMA polypeptide can be designed to include an IgG (e.g., IgG1) configuration having (a) a heavy chain comprising, consisting essentially of, or consisting of a heavy chain variable domain comprising SEQ ID NO:24, an Ig hinge, and constant domains (e.g., CH1, CH2, and CH3 domains) and (b) a light chain comprising, consisting essentially of, or consisting of a light chain variable domain comprising SEQ ID NO:32, a constant domain (e.g., a kappa or lambda constant domain), and an antigen binding domain having the ability to bind to a polypeptide expressed on the surface of an NK cell (e.g., an anti-human CD16a scFv or an anti-human NKG2A scFv).

In one embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (i) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications); and/or (ii) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications). An example of such a binder having these CDRs and the ability to bind to a PSMA polypeptide (e.g., a human PSMA polypeptide) includes, without limitation, the scFv set forth in FIGS. 2A and 2B.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and (a) a heavy chain variable domain having a CDR1 having the amino acid sequence set forth in SEQ ID NO:1 (or a variant of SEQ ID NO:1 with one or two amino acid modifications), a CDR2 having the amino acid sequence set forth in SEQ ID NO:2 (or a variant of SEQ ID NO:2 with one or two amino acid modifications), and a CDR3 having the amino acid sequence set forth in SEQ ID NO:3 (or a variant of SEQ ID NO:3 with one or two amino acid modifications) and/or (b) a light chain variable domain having a CDR1 having the amino acid sequence set forth in SEQ ID NO:9 (or a variant of SEQ ID NO:9 with one or two amino acid modifications), a CDR2 having the amino acid sequence set forth in SEQ ID NO:10 (or a variant of SEQ ID NO:10 with one or two amino acid modifications), and a CDR3 having the amino acid sequence set forth SEQ ID NO:11 (or a variant of SEQ ID NO:11 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:4 (or a variant of SEQ ID NO:4 with one, two, three, four, five, six, seven, eight, nine, ten, or more amino acid modifications), a framework region 2 having the amino acid sequence set forth in SEQ ID NO:5 (or a variant of SEQ ID NO:5 with one, two, three, four, five, six, seven, eight, nine, ten, or more amino acid modifications), a framework region 3 having the amino acid sequence set forth in SEQ ID NO:6 (or a variant of SEQ ID NO:6 with one, two, three, four, five, six, seven, eight, nine, ten, or more amino acid modifications), and a framework region 4 having the amino acid sequence set forth in SEQ ID NO:7 (or a variant of SEQ ID NO:7 with one, two, three, four, five, six, seven, eight, nine, ten, or more amino acid modifications) and/or (b) a light chain variable domain that includes a framework region 1 having the amino acid sequence set forth in SEQ ID NO:12 (or a variant of SEQ ID NO:12 with one, two, three, four, five, six, seven, eight, nine, ten, or more amino acid modifications), a framework region 2 having the amino acid sequence set forth in SEQ ID NO:13 (or a variant of SEQ ID NO:13 with one, two, three, four, five, six, seven, eight, nine, ten, or more amino acid modifications), a framework region 3 having the amino acid sequence set forth in SEQ ID NO:14 (or a variant of SEQ ID NO:14 with one, two, three, four, five, six, seven, eight, nine, ten, or more amino acid modifications), and a framework region 4 having the amino acid sequence set forth in SEQ ID NO:15 (or a variant of SEQ ID NO:15 with one, two, three, four, five, six, seven, eight, nine, ten, or more amino acid modifications).

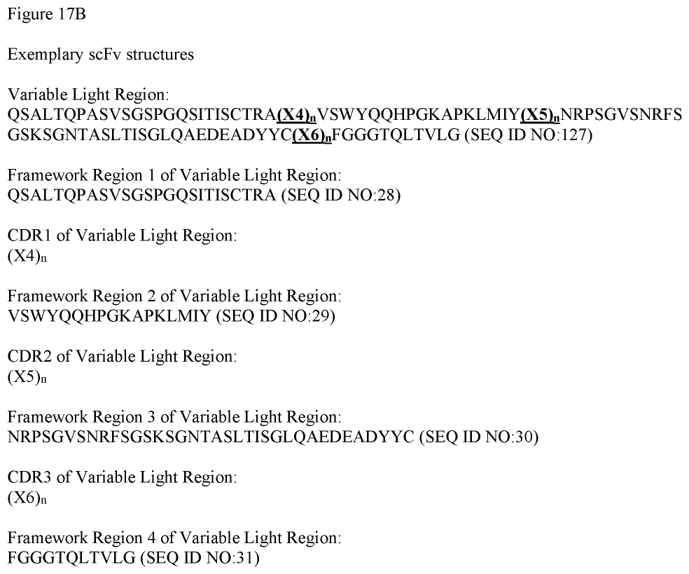

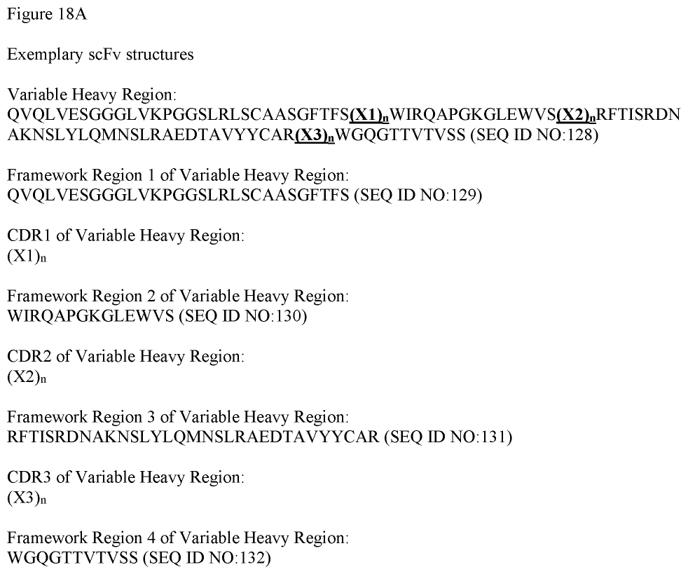

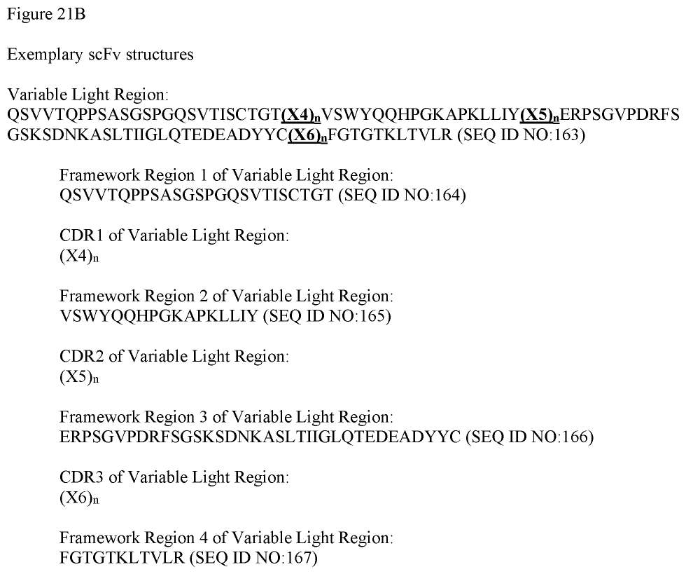

In some cases, a binder (e.g., an antibody, antigen binding fragment, antibody domain, a CAR, a cell engager, and/or an ADC) having any of the CDRs set forth in FIG. 2A or 2B can be designed to include framework regions as set forth in FIGS. 2A and 2B or can be designed to include one or more framework regions from another antibody, antibody fragment, or antibody domain. For example, a scFv can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:20 or a framework region 1 having the amino acid set forth in SEQ ID NO:129 or a framework region 1 having the amino acid set forth in SEQ ID NO:139 or a framework region 1 having the amino acid set forth in SEQ ID NO:149 or a framework region 1 having the amino acid set forth in SEQ ID NO:159. In some cases, an Fab can be designed to include the six CDRs set forth in FIGS. 2A and 2B and the framework regions set forth in FIGS. 2A and 2B. In some cases, an Fab can be designed to include the six CDRs set forth in FIGS. 2A and 2B and the framework regions set forth in FIGS. 2A and 2B except that framework region 1 having the amino acid set forth in SEQ ID NO:4 is replaced with a framework region 1 having the amino acid set forth in SEQ ID NO:20 or a framework region 1 having the amino acid set forth in SEQ ID NO:129 or a framework region 1 having the amino acid set forth in SEQ ID NO:139 or a framework region 1 having the amino acid set forth in SEQ ID NO:149 or a framework region 1 having the amino acid set forth in SEQ ID NO:159. In another example, a scFv can be designed to include the six CDRs set forth in FIGS. 2A and 2B and the framework regions set forth in FIGS. 17A and 17B, FIGS. 18A and 18B, FIGS. 19A and 19B, FIGS. 20A and 20B, or FIGS. 21A and 21B.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain that includes an amino acid sequence having at least 90 percent identity to the amino acid sequence set forth in SEQ ID NO:8 and/or (b) a light chain variable domain that includes an amino acid sequence having at least 90 percent identity to the amino acid sequence set forth in SEQ ID NO:16. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8 and/or (b) a light chain variable domain that includes an amino acid sequence having at least 90, 91, 92, 93, 94, 95, 96, 97, 98, or 99 percent identity to the amino acid sequence set forth in SEQ ID NO:16. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:8 and/or (b) a light chain variable domain that includes an amino acid sequence having 100 percent identity to the amino acid sequence set forth in SEQ ID NO:16.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain that includes an amino acid sequence having at least 90 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90 percent identity to the amino acid sequence set forth in SEQ ID NO:16, provided that the light chain variable domain includes the amino acid sequences set forth in SEQ ID NOs:9, 10, and 11. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:8, provided that the heavy chain variable domain includes the amino acid sequences set forth in SEQ ID NOs:1, 2, and 3, and/or (b) a light chain variable domain that includes an amino acid sequence having at least 90, 91, 92, 93, 94, 95, 96, 97, 98, or 99 percent identity to the amino acid sequence set forth in SEQ ID NO:16, provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain having the amino acid sequence set forth in SEQ ID NO:8 or the amino acid set forth in SEQ ID NO:8 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:16 or the amino acid set forth in SEQ ID NO:16 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a PSMA polypeptide (e.g., a human PSMA polypeptide), can include a heavy chain variable domain having the amino acid sequence set forth in SEQ ID NO:8 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:1, 2, and 3, and can include a light chain variable domain having the amino acid sequence set forth in SEQ ID NO:16 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:9, 10, and 11.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain comprising (i) a CDR1 that comprises, consists essentially of, or consists of the amino acid sequence set forth in SEQ ID NO:1, (ii) a CDR2 that comprises, consists essentially of, or consists of the amino acid sequence set forth in SEQ ID NO:2, and (iii) a CDR3 that comprises, consists essentially of, or consists of the amino acid sequence set forth in SEQ ID NO:3, and/or (b) a light chain variable domain comprising (i) a CDR1 that comprises, consists essentially of, or consists of the amino acid sequence set forth in SEQ ID NO:9, (ii) a CDR2 that comprises, consists essentially of, or consists of the amino acid sequence set forth in SEQ ID NO:10, and (iii) a CDR3 that comprises, consists essentially of, or consists of the amino acid sequence set forth in SEQ ID NO:11. As used herein, a “CDR1 that consists essentially of the amino acid sequence set forth in SEQ ID NO:1” is a CDR1 that has zero, one, or two amino acid substitutions within SEQ ID NO:1, that has zero, one, two, three, four, or five amino acid residues directly preceding SEQ ID NO:1, and/or that has zero, one, two, three, four, or five amino acid residues directly following SEQ ID NO:1, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:1 include, without limitation, those set forth in Table 1.

TABLE 1
Exemplary CDR1s that consist essentially of the
amino acid sequence set forth in SEQ ID NO: 1.

| Sequence | SEQ ID NO: |
|---|---|
| GYTFTGYY | 277 |
| GYSFTSYW | 278 |
| GYTFTIYG | 279 |
| GYSFTTYG | 280 |
| GYTFTSYA | 281 |
| GYTFTSYD | 282 |
| GYTFTSYG | 283 |
| GYTLTELS | 284 |
| GYTFTYRY | 285 |
| GYTFTCCS | 286 |

As used herein, a “CDR2 that consists essentially of the amino acid sequence set forth in SEQ ID NO:2” is a CDR2 that has zero, one, or two amino acid substitutions within SEQ TD NO:2, that has zero, one, two, three, four, or five amino acid residues directly preceding SEQ ID NO:2, and/or that has zero, one, two, three, four, or five amino acid residues directly following SEQ ID NO:2, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:2 include, without limitation, those set forth in Table 2.

TABLE 2
Exemplary CDR2s that consist essentially of the
amino acid sequence set forth in SEQ ID NO: 2.

| Sequence | SEQ ID NO: |
|---|---|
| IYWNDDK | 287 |
| IYSGGST | 288 |
| IYSGGSST | 289 |
| IYPGNSDT | 290 |
| IDPSDSYT | 291 |
| IYSGGSST | 292 |
| INSDGSST | 293 |
| ISSNGGST | 294 |
| IKDDGSQI | 295 |
| IQCDGSQI | 296 |

As used herein, a “CDR3 that consists essentially of the amino acid sequence set forth in SEQ ID NO:3” is a CDR3 that has zero, one, or two amino acid substitutions within SEQ ID NO:3, that has zero, one, two, three, four, or five amino acid residues directly preceding SEQ ID NO:3, and/or that has zero, one, two, three, four, or five amino acid residues directly following SEQ ID NO:3, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:3 include, without limitation, those set forth in Table 3.

TABLE 3
Exemplary CDR3s that consist essentially of the
amino acid sequence set forth in SEQ ID NO: 3.

| Sequence | SEQ ID NO: |
|---|---|
| ARQGGFYYGMDV | 297 |
| ARQGGYFYGMDV | 298 |
| ARQRGYYYGMDV | 299 |
| ARQGRYYFGMDV | 300 |
| ARQSGYYYGMDV | 301 |
| ARQGSYYYGMDV | 302 |
| ARQGGYYGGMDV | 303 |
| ARQGGYAYGMDV | 304 |
| ARQRGAYYGMDV | 305 |
| ARQGGAYYRMDV | 306 |

As used herein, a “CDR1 that consists essentially of the amino acid sequence set forth in SEQ ID NO:9” is a CDR1 that has zero, one, or two amino acid substitutions within SEQ ID NO:9, that has zero, one, two, three, four, or five amino acid residues directly preceding SEQ ID NO:9, and/or that has zero, one, two, three, four, or five amino acid residues directly following SEQ ID NO:9, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:9 include, without limitation, those set forth in Table 4.

TABLE 4
Exemplary CDR1s that consist essentially of the
amino acid sequence set forth in SEQ ID NO: 9.

| Sequence | SEQ ID NO: |
|---|---|
| SGYSNYK | 307 |
| SGSVSTSYY | 308 |
| SGINVGTYR | 309 |
| SLRSYY | 310 |
| SDINVGSYN | 311 |
| SGHSSYA | 312 |
| SGHSSYI | 313 |
| SEHSTYT | 314 |
| SDLSVGGKN | 315 |
| SGINLGSYR | 316 |

As used herein, a “CDR2 that consists essentially of the amino acid sequence set forth in SEQ ID NO:10” is a CDR2 that has zero, one, or two amino acid substitutions within SEQ ID NO:10, that has zero, one, two, three, four, or five amino acid residues directly preceding SEQ ID NO:10, and/or that has zero, one, two, three, four, or five amino acid residues directly following SEQ ID NO:10, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:10 include, without limitation, those set forth in Table 5.

TABLE 5
Exemplary CDR2s that consist essentially of the
amino acid sequence set forth in SEQ ID NO: 10.

| Sequence | SEQ ID NO: |
|---|---|
| YDD | 317 |
| GNS | 318 |
| KNN | 319 |
| EVS | 320 |
| EGS | 321 |
| EDS | 322 |
| STS | 323 |
| DTS | 324 |
| STN | 325 |
| GKN | 326 |

As used herein, a “CDR3 that consists essentially of the amino acid sequence set forth in SEQ ID NO:11” F is a CDR3 that has zero, one, or two amino acid substitutions within SEQ TD NO: 11, that has zero, one, two, three, four, or five amino acid residues directly preceding SEQ ID NO:11, and/or that has zero, one, two, three, four, or five amino acid residues directly following SEQ ID NO: 11, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:11 include, without limitation, those set forth in Table 6.

TABLE 6
Exemplary CDR3s that consist essentially of the
amino acid sequence set forth in SEQ ID NO: 11.

| Sequence | SEQ ID NO: |
|---|---|
| QSGDSRNHVV | 327 |
| QQYDSRNHVV | 328 |
| QKYDSRNHVV | 329 |
| QSYYSRNHVV | 330 |
| QSYDSRNQVV | 331 |
| QSYGSRNHVV | 332 |
| QSYASRNHVV | 333 |
| QYSDSRNHVV | 334 |
| QSYDDYRNHVV | 335 |
| QSYDSRYHVV | 336 |

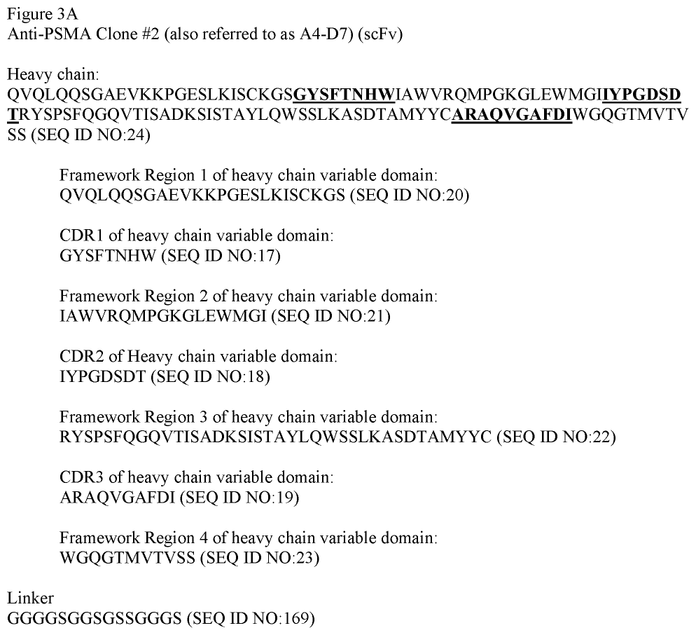

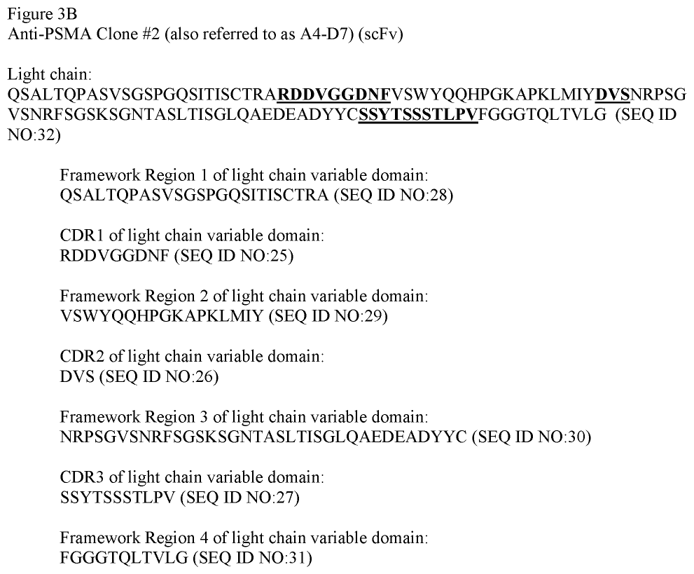

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (i) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications); and/or (ii) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications). An example of such a binder having these CDRs and the ability to bind to a PSMA polypeptide (e.g., a human PSMA polypeptide) includes, without limitation, the scFv set forth in FIGS. 3A and 3B.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having (a) a heavy chain variable domain having a CDR1 having the amino acid sequence set forth in SEQ ID NO:17 (or a variant of SEQ ID NO:17 with one or two amino acid modifications), a CDR2 having the amino acid sequence set forth in SEQ ID NO:18 (or a variant of SEQ ID NO:18 with one or two amino acid modifications), and a CDR3 having the amino acid sequence set forth in SEQ ID NO:19 (or a variant of SEQ ID NO:19 with one or two amino acid modifications) and/or (b) a light chain variable domain having a CDR1 having the amino acid sequence set forth in SEQ ID NO:25 (or a variant of SEQ ID NO:25 with one or two amino acid modifications), a CDR2 having the amino acid sequence set forth in SEQ ID NO:26 (or a variant of SEQ ID NO:26 with one or two amino acid modifications), and a CDR3 having the amino acid sequence set forth SEQ ID NO:27 (or a variant of SEQ ID NO:27 with one or two amino acid modifications) can include any appropriate framework regions. For example, such a binder (e.g., an antibody, antigen binding fragment, antibody domain, a CAR, a cell engager, and/or an ADC) can include (a) a heavy chain variable domain that includes a framework region 1 having the amino acid sequence set forth in SEQ ID NO:20 (or a variant of SEQ ID NO:20 with one, two, three, four, five, six, seven, eight, nine, ten, or more amino acid modifications), a framework region 2 having the amino acid sequence set forth in SEQ ID NO:21 (or a variant of SEQ ID NO:21 with one, two, three, four, five, six, seven, eight, nine, ten, or more amino acid modifications), a framework region 3 having the amino acid sequence set forth in SEQ ID NO:22 (or a variant of SEQ ID NO:22 with one, two, three, four, five, six, seven, eight, nine, ten, or more amino acid modifications), and a framework region 4 having the amino acid sequence set forth in SEQ ID NO:23 (or a variant of SEQ ID NO:23 with one, two, three, four, five, six, seven, eight, nine, ten, or more amino acid modifications) and/or (b) a light chain variable domain that includes a framework region 1 having the amino acid sequence set forth in SEQ ID NO:28 (or a variant of SEQ ID NO:28 with one, two, three, four, five, six, seven, eight, nine, ten, or more amino acid modifications), a framework region 2 having the amino acid sequence set forth in SEQ ID NO:29 (or a variant of SEQ ID NO:29 with one, two, three, four, five, six, seven, eight, nine, ten, or more amino acid modifications), a framework region 3 having the amino acid sequence set forth in SEQ ID NO:30 (or a variant of SEQ ID NO:30 with one, two, three, four, five, six, seven, eight, nine, ten, or more amino acid modifications), and a framework region 4 having the amino acid sequence set forth in SEQ ID NO:31 (or a variant of SEQ ID NO:31 with one, two, three, four, five, six, seven, eight, nine, ten, or more amino acid modifications).

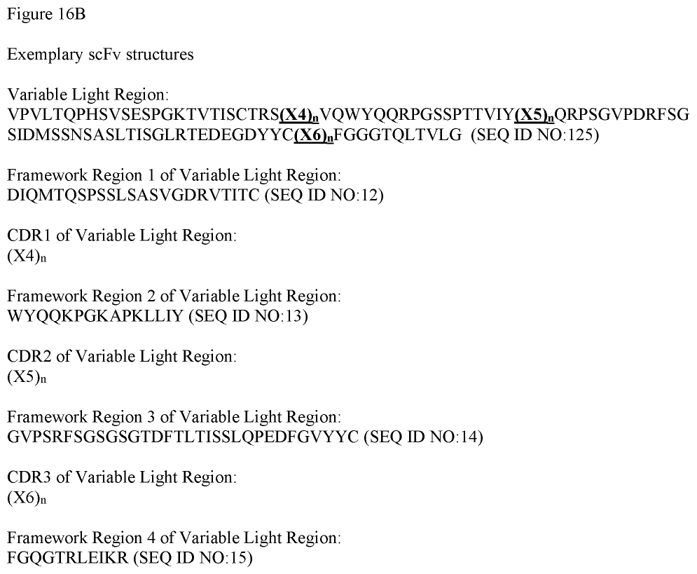

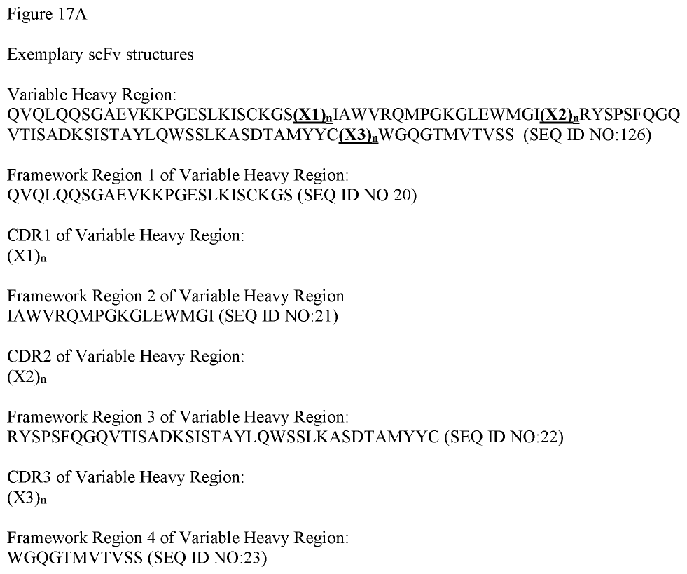

In some cases, a binder (e.g., an antibody, antigen binding fragment, antibody domain, a CAR, a cell engager, and/or an ADC) having any of the CDRs set forth in FIG. 3A or 3B can be designed to include framework regions as set forth in FIGS. 3A and 3B or can be designed to include one or more framework regions from another antibody or antibody fragment. For example, a scFv can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:4 or a framework region 1 having the amino acid set forth in SEQ ID NO:129 or a framework region 1 having the amino acid set forth in SEQ ID NO:139 or a framework region 1 having the amino acid set forth in SEQ ID NO:149 or a framework region 1 having the amino acid set forth in SEQ ID NO:159. In some cases, an Fab can be designed to include the six CDRs set forth in FIGS. 3A and 3B and the framework regions set forth in FIGS. 3A and 3B. In some cases, an Fab can be designed to include the six CDRs set forth in FIGS. 3A and 3B and the framework regions set forth in FIGS. 3A and 3B except that framework region 1 having the amino acid set forth in SEQ ID NO:20 is replaced with a framework region 1 having the amino acid set forth in SEQ ID NO:4 or a framework region 1 having the amino acid set forth in SEQ ID NO:129 or a framework region 1 having the amino acid set forth in SEQ ID NO:139 or a framework region 1 having the amino acid set forth in SEQ ID NO:149 or a framework region 1 having the amino acid set forth in SEQ ID NO:159. In another example, a scFv can be designed to include the six CDRs set forth in FIGS. 3A and 3B and the framework regions set forth in FIGS. 16A and 16B, FIGS. 18A and 18B, FIGS. 19A and 19B, FIGS. 20A and 20B, or FIGS. 21A and 21B.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain that includes an amino acid sequence having at least 90 percent identity to the amino acid sequence set forth in SEQ ID NO:24 and/or (b) a light chain variable domain that includes an amino acid sequence having at least 90 percent identity to the amino acid sequence set forth in SEQ ID NO:32. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24 and/or (b) a light chain variable domain that includes an amino acid sequence having at least 90, 91, 92, 93, 94, 95, 96, 97, 98, or 99 percent identity to the amino acid sequence set forth in SEQ ID NO:32. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:24 and/or (b) a light chain variable domain that includes an amino acid sequence having 100 percent identity to the amino acid sequence set forth in SEQ ID NO:32.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain that includes an amino acid sequence having at least 90 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90 percent identity to the amino acid sequence set forth in SEQ ID NO:32, provided that the light chain variable domain includes the amino acid sequences set forth in SEQ ID NOs:25, 26, and 27. For example, a binder (e.g., an antibody, antigen binding fragment, antibody domain, a CAR, a cell engager, and/or an ADC) provided herein can include (a) a heavy chain variable domain that includes an amino acid sequence having at least 90, 91, 92, 93, 94, 95, 96, 97, 98, or 99 percent identity to the amino acid sequence set forth in SEQ ID NO:24, provided that the heavy chain variable domain includes the amino acid sequences set forth in SEQ ID NOs:17, 18, and 19, and/or (b) a light chain variable domain that includes an amino acid sequence having at least 90, 91, 92, 93, 94, 95, 96, 97, 98, or 99 percent identity to the amino acid sequence set forth in SEQ ID NO:32, provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain having the amino acid sequence set forth in SEQ ID NO:24 or the amino acid set forth in SEQ ID NO:24 with one, two, three, four, five, six, seven, eight, nine, or 10 amino acid modifications (e.g., amino acid substitutions, amino acid deletions, and/or amino acid additions) and/or (b) a light chain variable domain that includes the amino acid sequence set forth in SEQ ID NO:32 or the amino acid set forth in SEQ ID NO:32 with one, two, three, four, five, six, seven, eight, nine, or 10 amino acid modifications (e.g., amino acid substitutions, amino acid deletions, and/or amino acid additions). For example, an antibody or antigen binding fragment provided herein can have the ability to bind to a PSMA polypeptide (e.g., a human PSMA polypeptide), can include a heavy chain variable domain having the amino acid sequence set forth in SEQ ID NO:24 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:17, 18, and 19, and can include a light chain variable domain having the amino acid sequence set forth in SEQ ID NO:32 with one, two, three, four, five, six, seven, eight, nine, or 10 amino acid modifications (e.g., amino acid substitutions, amino acid deletions, and/or amino acid additions), provided that the light chain variable domain includes the amino acid sequences set forth in SEQ ID NOs:25, 26, and 27.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include (a) a heavy chain variable domain comprising (i) a CDR1 that comprises, consists essentially of, or consists of the amino acid sequence set forth in SEQ ID NO:17, (ii) a CDR2 that comprises, consists essentially of, or consists of the amino acid sequence set forth in SEQ ID NO:18, and (iii) a CDR3 that comprises, consists essentially of, or consists of the amino acid sequence set forth in SEQ ID NO:19, and/or (b) a light chain variable domain comprising (i) a CDR1 that comprises, consists essentially of, or consists of the amino acid sequence set forth in SEQ ID NO:25, (ii) a CDR2 that comprises, consists essentially of, or consists of the amino acid sequence set forth in SEQ ID NO:26, and (iii) a CDR3 that comprises, consists essentially of, or consists of the amino acid sequence set forth in SEQ ID NO:27. As used herein, a “CDR1 that consists essentially of the amino acid sequence set forth in SEQ ID NO:17” is a CDR1 that has zero, one, or two amino acid substitutions within SEQ ID NO:17, that has zero, one, two, three, four, or five amino acid residues directly preceding SEQ ID NO:17, and/or that has zero, one, two, three, four, or five amino acid residues directly following SEQ ID NO:17, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:17 include, without limitation, those set forth in Table 7.

TABLE 7
Exemplary CDR1s that consist essentially of the
amino acid sequence set forth in SEQ ID NO: 17.

| Sequence | SEQ ID NO: |
|---|---|
| GYSFTTYG | 337 |
| GYTFTIYG | 338 |
| GYTFTSYA | 339 |
| GYSFTSYW | 340 |
| GYSISSGYY | 341 |
| GYSISSSNW | 342 |
| GGSISSSNW | 343 |
| GGSISSGGYS | 344 |
| GGSISSSSYY | 345 |
| GGSISSYY | 346 |

As used herein, a “CDR2 that consists essentially of the amino acid sequence set forth in SEQ ID NO:18” is a CDR2 that has zero, one, or two amino acid substitutions within SEQ ID NO:18, that has zero, one, two, three, four, or five amino acid residues directly preceding SEQ ID NO:18, and/or that has zero, one, two, three, four, or five amino acid residues directly following SEQ ID NO:18, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:18 include, without limitation, those set forth in Table 8.

TABLE 8
Exemplary CDR2s that consist essentially of the
amino acid sequence set forth in SEQ ID NO: 18.

| Sequence | SEQ ID NO: |
|---|---|
| IYWNDDK | 347 |
| IYSGGST | 348 |
| IYSGGSST | 349 |
| IYPGNSDT | 350 |
| IDPSDSYT | 351 |
| IYSGGSST | 352 |
| INSDGSST | 353 |
| ISSNGGST | 354 |
| IKDDGSQI | 355 |
| IQCDGSQI | 356 |

As used herein, a “CDR3 that consists essentially of the amino acid sequence set forth in SEQ ID NO:19” is a CDR3 that has zero, one, or two amino acid substitutions within SEQ ID NO:19, that has zero, one, two, three, four, or five amino acid residues directly preceding SEQ ID NO:19, and/or that has zero, one, two, three, four, or five amino acid residues directly following SEQ ID NO:19, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:19 include, without limitation, those set forth in Table 9.

TABLE 9
Exemplary CDR3s that consist essentially of the
amino acid sequence set forth in SEQ ID NO: 19.

| Sequence | SEQ ID NO: |
|---|---|
| ARALVGAFDI | 357 |
| ARARVGAFDI | 358 |
| ARGQVGAFDI | 359 |
| ARAQVGSFDI | 360 |
| ARAQVGAFLI | 361 |
| ARAQTGAFDI | 362 |
| ARSQVGAFDI | 363 |
| ARAQVWAFDI | 364 |
| ARAQVGNFDI | 365 |
| ARAQVGLFDI | 366 |

As used herein, a “CDR1 that consists essentially of the amino acid sequence set forth in SEQ ID NO:25” is a CDR1 that has zero, one, or two amino acid substitutions within SEQ ID NO:25, that has zero, one, two, three, four, or five amino acid residues directly preceding SEQ ID NO:25, and/or that has zero, one, two, three, four, or five amino acid residues directly following SEQ ID NO:25, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:25 include, without limitation, those set forth in Table 10.

TABLE 10
Exemplary CDR1s that consist essentially of the
amino acid sequence set forth in SEQ ID NO: 25.

| Sequence | SEQ ID NO: |
|---|---|
| SSDVGGYNY | 367 |
| SSDVGSYNR | 368 |
| SSDVGSYNL | 369 |
| SSDVGDYDH | 370 |
| SSDIGGYDL | 371 |
| SSDVGSYDY | 372 |
| SSNTGTGYN | 373 |
| SDINVGSYN | 374 |
| SGINVGTYR | 375 |
| SGINLGSYR | 376 |

As used herein, a “CDR2 that consists essentially of the amino acid sequence set forth in SEQ ID NO:26” is a CDR2 that has zero, one, or two amino acid substitutions within SEQ ID NO:26, that has zero, one, two, three, four, or five amino acid residues directly preceding SEQ ID NO:26, and/or that has zero, one, two, three, four, or five amino acid residues directly following SEQ ID NO:26, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:26 include, without limitation, those set forth in Table 11.

TABLE 11
Exemplary CDR2s that consist essentially of the
amino acid sequence set forth in SEQ ID NO: 26.

| Sequence | SEQ ID NO: |
|---|---|
| EVS | 377 |
| EGS | 378 |
| DVA | 379 |
| DNN | 380 |
| YDD | 381 |
| YDS | 382 |
| SDS | 383 |
| DSS | 384 |
| DTS | 385 |
| STS | 386 |

As used herein, a “CDR3 that consists essentially of the amino acid sequence set forth in SEQ ID NO:27” is a CDR3 that has zero, one, or two amino acid substitutions within SEQ TD NO:27, that has zero, one, two, three, four, or five amino acid residues directly preceding SEQ ID NO:27, and/or that has zero, one, two, three, four, or five amino acid residues directly following SEQ ID NO:27, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:27 include, without limitation, those set forth in Table 12.

TABLE 12
Exemplary CDR3s that consist essentially of the
amino acid sequence set forth in SEQ ID NO: 27.

| Sequence | SEQ ID NO: |
|---|---|
| SRYTSSSTLPV | 387 |
| SDYTSSSTLPV | 388 |
| SSYTSSNTLPV | 389 |
| SSYTGSSTLPV | 390 |
| SSYTSDDTLPV | 391 |
| SSYTSGSTLPV | 392 |
| SSYTSSSTWPV | 393 |
| SSYLSSSTLPV | 394 |
| SSYTSSSTLPV | 395 |
| SSYTSSSTLPV | 396 |

In one embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:33 (or a variant of SEQ ID NO:33 with one or two amino acid modifications), a CDR2 having the amino acid sequence set forth in SEQ ID NO:34 (or a variant of SEQ ID NO:34 with one or two amino acid modifications), and a CDR3 having the amino acid sequence set forth in SEQ ID NO:35 (or a variant of SEQ ID NO:35 with one or two amino acid modifications). An example of such a binder having these CDRs and the ability to bind to a PSMA polypeptide (e.g., a human PSMA polypeptide) includes, without limitation, the VH domain set forth in FIG. 4.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:33 (or a variant of SEQ ID NO:33 with one or two amino acid modifications), a CDR2 having the amino acid sequence set forth in SEQ ID NO:34 (or a variant of SEQ ID NO:34 with one or two amino acid modifications), and a CDR3 having the amino acid sequence set forth in SEQ ID NO:35 (or a variant of SEQ ID NO:35 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:36 (or a variant of SEQ ID NO:36 with one, two, three, four, five, six, seven, eight, nine, ten, or more amino acid modifications), a framework region 2 having the amino acid sequence set forth in SEQ ID NO:37 (or a variant of SEQ ID NO:37 with one, two, three, four, five, six, seven, eight, nine, ten, or more amino acid modifications), a framework region 3 having the amino acid sequence set forth in SEQ ID NO:38 (or a variant of SEQ ID NO:38 with one, two, three, four, five, six, seven, eight, nine, ten, or more amino acid modifications), and a framework region 4 having the amino acid sequence set forth in SEQ ID NO:39 (or a variant of SEQ ID NO:39 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 4 can be designed to include framework regions as set forth in FIG. 4 or can be designed to include one or more framework regions from another antibody or antibody fragment. For example, an antibody domain (e.g., a VH domain) can be designed to include the three CDRs set forth in FIG. 4 and the framework regions set forth in FIG. 4 except that framework region 1 having the amino acid set forth in SEQ ID NO:36 is replaced with a framework region 1 having the amino acid set forth in SEQ ID NO:52, a framework region 1 having the amino acid set forth in SEQ ID NO:60, or a framework region 1 having the amino acid set forth in SEQ ID NO:92. In another example, an Fab or scFv can be designed to include (a) the three CDRs set forth in FIG. 4, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:40. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:40. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:40.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:40, provided that the heavy chain variable domain includes the amino acid sequences set forth in SEQ ID NOs:33, 34, and 35. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:40, provided that the heavy chain variable domain includes the amino acid sequences set forth in SEQ ID NOs:33, 34, and 35.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:40 or the amino acid set forth in SEQ ID NO:40 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:40 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:33, 34, and 35.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:33, (ii) a CDR2 that comprises, consists essentially of, or consists of the amino acid sequence set forth in SEQ ID NO:34, and (iii) a CDR3 that comprises, consists essentially of, or consists of the amino acid sequence set forth in SEQ ID NO:35. As used herein, a “CDR1 that consists essentially of the amino acid sequence set forth in SEQ ID NO:33” is a CDR1 that has zero, one, or two amino acid substitutions within SEQ ID NO:33, that has zero, one, two, three, four, or five amino acid residues directly preceding SEQ ID NO:33, and/or that has zero, one, two, three, four, or five amino acid residues directly following SEQ ID NO:33, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:33 include, without limitation, those set forth in Table 13.

TABLE 13
Exemplary CDR1s that consist essentially of the
amino acid sequence set forth in SEQ ID NO: 33.

| Sequence | SEQ ID NO: |
|---|---|
| GFTFSSYW | 397 |
| GFTFDDYA | 398 |
| GFTFSDYY | 399 |
| GFTFSSYD | 400 |
| GFTFSNAW | 401 |
| GFTFSNSD | 402 |
| GFTFSNSD | 403 |
| GFTFDDYG | 404 |
| GFTFSSYS | 405 |
| GFTFSSYG | 406 |

As used herein, a “CDR2 that consists essentially of the amino acid sequence set forth in SEQ ID NO:34” is a CDR2 that has zero, one, or two amino acid substitutions within SEQ ID NO:34, that has zero, one, two, three, four, or five amino acid residues directly preceding SEQ ID NO:34, and/or that has zero, one, two, three, four, or five amino acid residues directly following SEQ ID NO:34, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:34 include, without limitation, those set forth in Table 14.

TABLE 14
Exemplary CDR2s that consist essentially of the
amino acid sequence set forth in SEQ ID NO: 34.

| Sequence | SEQ ID NO: |
|---|---|
| IKQDGSEK | 407 |
| ISWNSGSI | 408 |
| ISSSGSTI | 409 |
| IGTAGDT | 410 |
| IKSKTDGGTT | 411 |
| VSWNGSRT | 412 |
| INWNGGST | 413 |
| ISSSSSYI | 414 |
| ISGSGGST | 415 |
| IQCDGSQI | 416 |

As used herein, a “CDR3 that consists essentially of the amino acid sequence set forth in SEQ ID NO:35” is a CDR3 that has zero, one, or two amino acid substitutions within SEQ ID NO:35, that has zero, one, two, three, four, or five amino acid residues directly preceding SEQ ID NO:35, and/or that has zero, one, two, three, four, or five amino acid residues directly following SEQ ID NO:35, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:35 include, without limitation, those set forth in Table 15.

TABLE 15
Exemplary CDR3s that consist essentially of the
amino acid sequence set forth in SEQ ID NO: 35.

| Sequence | SEQ ID NO: |
|---|---|
| VGRSSTSITPV | 417 |
| VGRNSTSITPV | 418 |
| VGRYSTSITPV | 419 |
| VGRQSTSITPV | 420 |
| VGRGSLSITPV | 421 |
| VGRGSCSITPV | 422 |
| VGRGSISITPV | 423 |
| VGRGSTSILPV | 424 |
| VGRGSTSLTPV | 425 |
| VGRGSNSITPV | 426 |

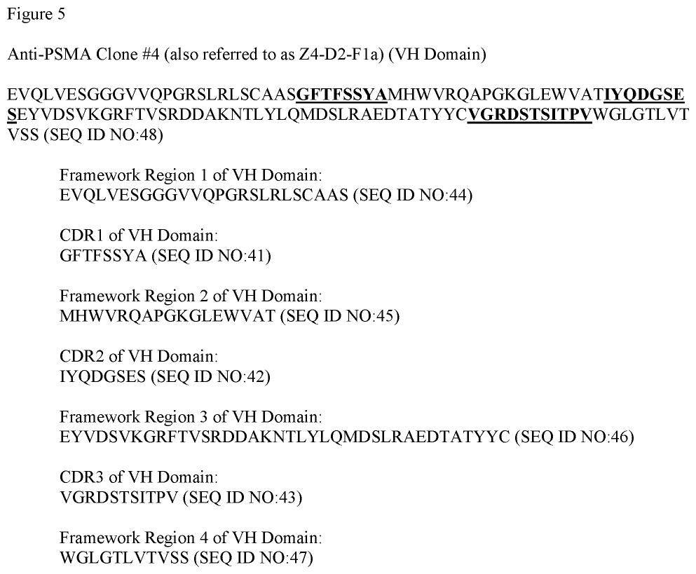

In one embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:41 (or a variant of SEQ ID NO:41 with one or two amino acid modifications), a CDR2 having the amino acid sequence set forth in SEQ ID NO:42 (or a variant of SEQ ID NO:42 with one or two amino acid modifications), and a CDR3 having the amino acid sequence set forth in SEQ ID NO:43 (or a variant of SEQ ID NO:43 with one or two amino acid modifications). An example of such a binder having these CDRs and the ability to bind to a PSMA polypeptide (e.g., a human PSMA polypeptide) includes, without limitation, the VH domain set forth in FIG. 5.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:41 (or a variant of SEQ ID NO:41 with one or two amino acid modifications), a CDR2 having the amino acid sequence set forth in SEQ ID NO:42 (or a variant of SEQ ID NO:42 with one or two amino acid modifications), and a CDR3 having the amino acid sequence set forth in SEQ ID NO:43 (or a variant of SEQ ID NO:43 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:44 (or a variant of SEQ ID NO:44 with one, two, three, four, five, six, seven, eight, nine, ten, or more amino acid modifications), a framework region 2 having the amino acid sequence set forth in SEQ ID NO:45 (or a variant of SEQ ID NO:45 with one, two, three, four, five, six, seven, eight, nine, ten, or more amino acid modifications), a framework region 3 having the amino acid sequence set forth in SEQ ID NO:46 (or a variant of SEQ ID NO:46 with one, two, three, four, five, six, seven, eight, nine, ten, or more amino acid modifications), and a framework region 4 having the amino acid sequence set forth in SEQ ID NO:47 (or a variant of SEQ ID NO:47 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 5 can be designed to include framework regions as set forth in FIG. 5 or can be designed to include one or more framework regions from another antibody or antibody fragment. For example, an antibody domain (e.g., a VH domain) can be designed to include the three CDRs set forth in FIG. 5 and the framework regions set forth in FIG. 5 except that framework region 1 having the amino acid set forth in SEQ ID NO:44 is replaced with a framework region 1 having the amino acid set forth in SEQ ID NO:52, a framework region 1 having the amino acid set forth in SEQ ID NO:60, or a framework region 1 having the amino acid set forth in SEQ ID NO:92. In another example, an Fab or scFv can be designed to include (a) the three CDRs set forth in FIG. 5, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:48. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:48. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:48.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:48, provided that the heavy chain variable domain includes the amino acid sequences set forth in SEQ ID NOs:41, 42, and 43. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:48, provided that the heavy chain variable domain includes the amino acid sequences set forth in SEQ ID NOs:41, 42, and 43.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:48 or the amino acid set forth in SEQ ID NO:48 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:48 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:41, 42, and 43.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:41, (ii) a CDR2 that comprises, consists essentially of, or consists of the amino acid sequence set forth in SEQ ID NO:42, and (iii) a CDR3 that comprises, consists essentially of, or consists of the amino acid sequence set forth in SEQ ID NO:43. As used herein, a “CDR1 that consists essentially of the amino acid sequence set forth in SEQ ID NO:41” is a CDR1 that has zero, one, or two amino acid substitutions within SEQ ID NO:41, that has zero, one, two, three, four, or five amino acid residues directly preceding SEQ ID NO:41, and/or that has zero, one, two, three, four, or five amino acid residues directly following SEQ ID NO:41, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:41 include, without limitation, those set forth in Table 16.

TABLE 16
Exemplary CDR1s that consist essentially of the
amino acid sequence set forth in SEQ ID NO: 41.

| Sequence | SEQ ID NO: |
|---|---|
| GFTFSSYW | 427 |
| GFTFDDYA | 428 |
| GFTFSDYY | 429 |
| GFTFSSYD | 430 |
| GFTFSNAW | 431 |
| GFTFSNSD | 432 |
| GFTFSNSD | 433 |
| GFTFDDYG | 434 |
| GFTFSSYS | 435 |
| GFTFSSYG | 436 |

As used herein, a “CDR2 that consists essentially of the amino acid sequence set forth in SEQ ID NO:42” is a CDR2 that has zero or one amino acid substitutions within SEQ ID NO:42, that has zero, one, two, three, four, or five amino acid residues directly preceding SEQ ID NO:42, and/or that has zero, one, two, three, four, or five amino acid residues directly following SEQ ID NO:42, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:42 include, without limitation, those set forth in Table 17.

TABLE 17
Exemplary CDR2s that consist essentially of the
amino acid sequence set forth in SEQ ID NO: 42.

| Sequence | SEQ ID NO: |
|---|---|
| IKQDGSEK | 437 |
| ISWNSGSI | 438 |
| ISSSGSTI | 439 |
| IGTAGDT | 440 |
| IKSKTDGGTT | 441 |
| VSWNGSRT | 442 |
| INWNGGST | 443 |
| ISSSSSYI | 444 |
| ISGSGGST | 445 |
| IQCDGSQI | 446 |

As used herein, a “CDR3 that consists essentially of the amino acid sequence set forth in SEQ ID NO:43” is a CDR3 that has zero, one, or two amino acid substitutions within SEQ TD NO:43, that has zero, one, two, three, four, or five amino acid residues directly preceding SEQ ID NO:43, and/or that has zero, one, two, three, four, or five amino acid residues directly following SEQ ID NO:43, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:43 include, without limitation, those set forth in Table 18.

TABLE 18
Exemplary CDR3s that consist essentially of the
amino acid sequence set forth in SEQ ID NO: 43.

| Sequence | SEQ ID NO: |
|---|---|
| VGRSSTSITPV | 447 |
| VGRNSTSITPV | 448 |
| VGRYSTSITPV | 449 |
| VGRQSTSITPV | 450 |
| VGRGSLSITPV | 451 |
| VGRGSCSITPV | 452 |
| VGRGSISITPV | 453 |
| VGRGSTSILPV | 454 |
| VGRGSTSLTPV | 455 |
| VGRGSNSITPV | 456 |

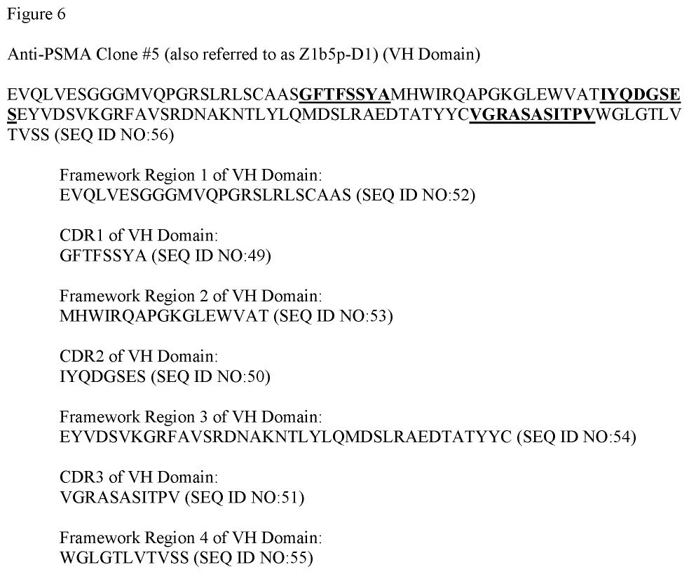

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:49 (or a variant of SEQ ID NO:49 with one or two amino acid modifications), a CDR2 having the amino acid sequence set forth in SEQ ID NO:50 (or a variant of SEQ ID NO:50 with one or two amino acid modifications), and a CDR3 having the amino acid sequence set forth in SEQ ID NO:51 (or a variant of SEQ ID NO:51 with one or two amino acid modifications). An example of such a binder having these CDRs and the ability to bind to a PSMA polypeptide (e.g., a human PSMA polypeptide) includes, without limitation, the VH domain set forth in FIG. 6.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:49 (or a variant of SEQ ID NO:49 with one or two amino acid modifications), a CDR2 having the amino acid sequence set forth in SEQ ID NO:50 (or a variant of SEQ ID NO:50 with one or two amino acid modifications), and a CDR3 having the amino acid sequence set forth in SEQ ID NO:51 (or a variant of SEQ ID NO:51 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:52 (or a variant of SEQ ID NO:52 with one, two, three, four, five, six, seven, eight, nine, ten, or more amino acid modifications), a framework region 2 having the amino acid sequence set forth in SEQ ID NO:53 (or a variant of SEQ ID NO:53 with one, two, three, four, five, six, seven, eight, nine, ten, or more amino acid modifications), a framework region 3 having the amino acid sequence set forth in SEQ ID NO:54 (or a variant of SEQ ID NO:54 with one, two, three, four, five, six, seven, eight, nine, ten, or more amino acid modifications), and a framework region 4 having the amino acid sequence set forth in SEQ ID NO:55 (or a variant of SEQ ID NO:55 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 6 can be designed to include framework regions as set forth in FIG. 6 or can be designed to include one or more framework regions from another antibody or antibody fragment. For example, an antibody domain (e.g., a VH domain) can be designed to include the three CDRs set forth in FIG. 6 and the framework regions set forth in FIG. 6 except that framework region 1 having the amino acid set forth in SEQ ID NO:52 is replaced with a framework region 1 having the amino acid set forth in SEQ ID NO:36, a framework region 1 having the amino acid set forth in SEQ ID NO:60, or a framework region 1 having the amino acid set forth in SEQ ID NO:92. In another example, an Fab or scFv can be designed to include (a) the three CDRs set forth in FIG. 6, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:56. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:56. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:56.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:56, provided that the heavy chain variable domain includes the amino acid sequences set forth in SEQ ID NOs:49, 50, and 51. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:56, provided that the heavy chain variable domain includes the amino acid sequences set forth in SEQ ID NOs:49, 50, and 51.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:56 or the amino acid set forth in SEQ ID NO:56 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:56 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:49, 50, and 51.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:49, (ii) a CDR2 that comprises, consists essentially of, or consists of the amino acid sequence set forth in SEQ ID NO:50, and (iii) a CDR3 that comprises, consists essentially of, or consists of the amino acid sequence set forth in SEQ ID NO:51. As used herein, a “CDR1 that consists essentially of the amino acid sequence set forth in SEQ ID NO:49” is a CDR1 that has zero, one, or two amino acid substitutions within SEQ ID NO:49, that has zero, one, two, three, four, or five amino acid residues directly preceding SEQ ID NO:49, and/or that has zero, one, two, three, four, or five amino acid residues directly following SEQ ID NO:49, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:49 include, without limitation, those set forth in Table 19.

TABLE 19
Exemplary CDR1s that consist essentially of the
amino acid sequence set forth in SEQ ID NO: 49.

| Sequence | SEQ ID NO: |
|---|---|
| GFTFSSYW | 457 |
| GFTFDDYA | 458 |
| GFTFSDYY | 459 |
| GFTFSSYD | 460 |
| GFTFSNAW | 461 |
| GFTFSNSD | 462 |
| GFTFSNSD | 463 |
| GFTFDDYG | 464 |
| GFTFSSYS | 465 |
| GFTFSSYG | 466 |

As used herein, a “CDR2 that consists essentially of the amino acid sequence set forth in SEQ ID NO:50” is a CDR2 that has zero, one, or two amino acid substitutions within SEQ ID NO:50, that has zero, one, two, three, four, or five amino acid residues directly preceding SEQ ID NO:50, and/or that has zero, one, two, three, four, or five amino acid residues directly following SEQ ID NO:50, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:50 include, without limitation, those set forth in Table 20.

TABLE 20
Exemplary CDR2s that consist essentially of the
amino acid sequence set forth in SEQ ID NO: 50.

| Sequence | SEQ ID NO: |
|---|---|
| IKQDGSEK | 467 |
| ISWNSGSI | 468 |
| ISSSGSTI | 469 |
| IGTAGDT | 470 |
| IKSKTDGGTT | 471 |
| VSWNGSRT | 472 |
| INWNGGST | 473 |
| ISSSSSYI | 474 |
| ISGSGGST | 475 |
| IQCDGSQI | 476 |

As used herein, a “CDR3 that consists essentially of the amino acid sequence set forth in SEQ ID NO:51” is a CDR3 that has zero, one, or two amino acid substitutions within SEQ ID NO:51, that has zero, one, two, three, four, or five amino acid residues directly preceding SEQ ID NO:51, and/or that has zero, one, two, three, four, or five amino acid residues directly following SEQ ID NO:51, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:51 include, without limitation, those set forth in Table 21.

TABLE 21
Exemplary CDR3s that consist essentially of the
amino acid sequence set forth in SEQ ID NO: 51.

| Sequence | SEQ ID NO: |
|---|---|
| VGRSSTSITPV | 477 |
| VGRNSTSITPV | 478 |
| VGRYSTSITPV | 479 |
| VGRQSTSITPV | 480 |
| VGRGSLSITPV | 481 |
| VGRGSCSITPV | 482 |
| VGRGSISITPV | 483 |
| VGRGSTSILPV | 484 |
| VGRGSTSLTPV | 485 |
| VGRGSNSITPV | 486 |

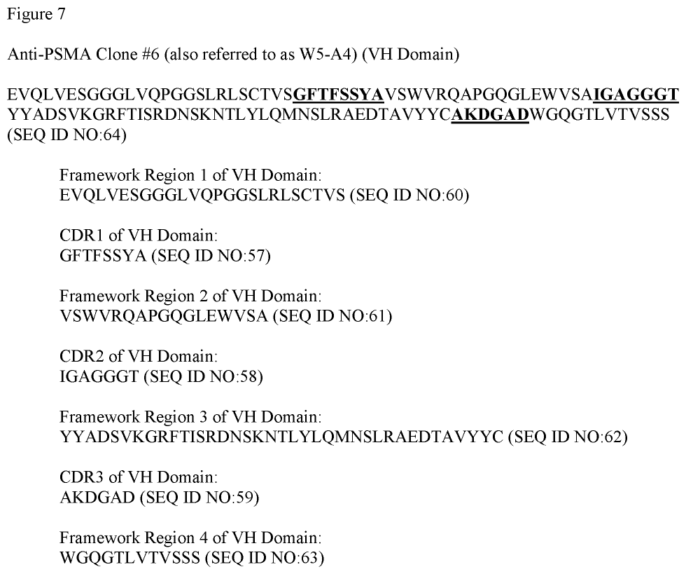

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:57 (or a variant of SEQ ID NO:57 with one or two amino acid modifications), a CDR2 having the amino acid sequence set forth in SEQ ID NO:58 (or a variant of SEQ ID NO:58 with one or two amino acid modifications), and a CDR3 having the amino acid sequence set forth in SEQ ID NO:59 (or a variant of SEQ ID NO:59 with one or two amino acid modifications). An example of such a binder having these CDRs and the ability to bind to a PSMA polypeptide (e.g., a human PSMA polypeptide) includes, without limitation, the VH domain set forth in FIG. 7.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:57 (or a variant of SEQ ID NO:57 with one or two amino acid modifications), a CDR2 having the amino acid sequence set forth in SEQ ID NO:58 (or a variant of SEQ ID NO:58 with one or two amino acid modifications), and a CDR3 having the amino acid sequence set forth in SEQ ID NO:59 (or a variant of SEQ ID NO:59 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:60 (or a variant of SEQ ID NO:60 with one, two, three, four, five, six, seven, eight, nine, ten, or more amino acid modifications), a framework region 2 having the amino acid sequence set forth in SEQ ID NO:61 (or a variant of SEQ ID NO:61 with one, two, three, four, five, six, seven, eight, nine, ten, or more amino acid modifications), a framework region 3 having the amino acid sequence set forth in SEQ ID NO:62 (or a variant of SEQ ID NO:62 with one, two, three, four, five, six, seven, eight, nine, ten, or more amino acid modifications), and a framework region 4 having the amino acid sequence set forth in SEQ ID NO:63 (or a variant of SEQ ID NO:63 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 7 can be designed to include framework regions as set forth in FIG. 7 or can be designed to include one or more framework regions from another antibody or antibody fragment. For example, an antibody domain (e.g., a VH domain) can be designed to include the three CDRs set forth in FIG. 7 and the framework regions set forth in FIG. 7 except that framework region 1 having the amino acid set forth in SEQ ID NO:60 is replaced with a framework region 1 having the amino acid set forth in SEQ ID NO:36, a framework region 1 having the amino acid set forth in SEQ ID NO:52, or a framework region 1 having the amino acid set forth in SEQ ID NO:92. In another example, an Fab or scFv can be designed to include (a) the three CDRs set forth in FIG. 7, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:64. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:64. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:64.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:64, provided that the heavy chain variable domain includes the amino acid sequences set forth in SEQ ID NOs:57, 58, and 59. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:64, provided that the heavy chain variable domain includes the amino acid sequences set forth in SEQ ID NOs:57, 58, and 59.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:64 or the amino acid set forth in SEQ ID NO:64 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:64 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:57, 58, and 59.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:57, (ii) a CDR2 that comprises, consists essentially of, or consists of the amino acid sequence set forth in SEQ ID NO:58, and (iii) a CDR3 that comprises, consists essentially of, or consists of the amino acid sequence set forth in SEQ ID NO:59. As used herein, a “CDR1 that consists essentially of the amino acid sequence set forth in SEQ ID NO:57” is a CDR1 that has zero, one, or two amino acid substitutions within SEQ ID NO:57, that has zero, one, two, three, four, or five amino acid residues directly preceding SEQ ID NO:57, and/or that has zero, one, two, three, four, or five amino acid residues directly following SEQ ID NO:57, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:57 include, without limitation, those set forth in Table 22.

TABLE 22
Exemplary CDR1s that consist essentially of the
amino acid sequence set forth in SEQ ID NO: 57.

| Sequence | SEQ ID NO: |
|---|---|
| GFTFSSYW | 487 |
| GFTFDDYA | 488 |
| GFTFSDYY | 489 |
| GFTFSSYD | 490 |
| GFTFSNAW | 491 |
| GFTFSNSD | 492 |
| GFTFSNSD | 493 |
| GFTFDDYG | 494 |
| GFTFSSYS | 495 |
| GFTFSSYG | 496 |

As used herein, a “CDR2 that consists essentially of the amino acid sequence set forth in SEQ ID NO:58” is a CDR2 that has zero, one, or two amino acid substitutions within SEQ ID NO:58, that has zero, one, two, three, four, or five amino acid residues directly preceding SEQ ID NO:58, and/or that has zero, one, two, three, four, or five amino acid residues directly following SEQ ID NO:58, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:58 include, without limitation, those set forth in Table 23.

TABLE 23
Exemplary CDR2s that consist essentially of the
amino acid sequence set forth in SEQ ID NO: 58.

| Sequence | SEQ ID NO: |
|---|---|
| IKSKTDGGTT | 147 |
| INWNGGST | 498 |
| ISGSGGST | 499 |
| IGTAGDT | 500 |
| VNPNGGST | 501 |
| INSDGSST | 502 |
| IYSGGSST | 503 |
| IYHSGST | 504 |
| INPSGGST | 505 |
| INPNSGGT | 506 |

As used herein, a “CDR3 that consists essentially of the amino acid sequence set forth in SEQ ID NO:59” is a CDR3 that has zero, one, or two amino acid substitutions within SEQ ID NO:59, that has zero, one, two, three, four, or five amino acid residues directly preceding SEQ ID NO:59, and/or that has zero, one, two, three, four, or five amino acid residues directly following SEQ ID NO:59, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:59 include, without limitation, those set forth in Table 24.

TABLE 24
Exemplary CDR3s that consist essentially of the
amino acid sequence set forth in SEQ ID NO: 59.

| Sequence | SEQ ID NO: |
|---|---|
| ARDGAD | 507 |
| AKNGAD | 508 |
| AKDGKD | 509 |
| AKDGND | 510 |
| ARDGND | 511 |
| ARDGKD | 512 |
| AKNGAD | 513 |
| AKDGSD | 514 |
| AKDGTD | 515 |
| ARDSAD | 516 |

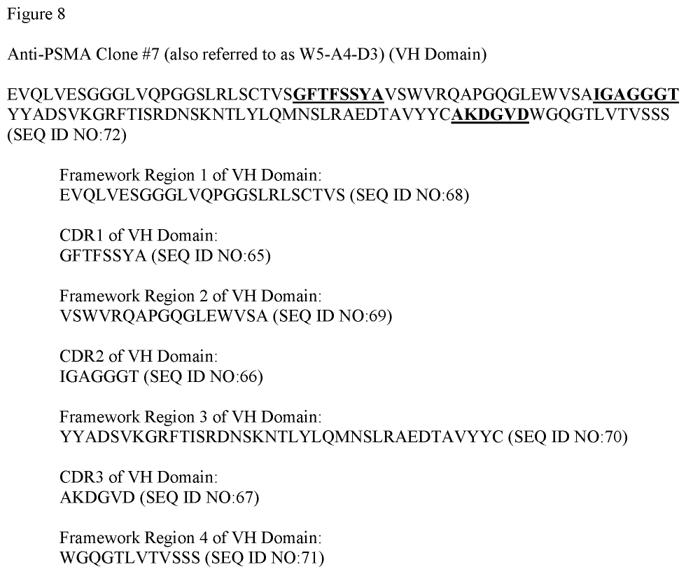

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:65 (or a variant of SEQ ID NO:65 with one or two amino acid modifications), a CDR2 having the amino acid sequence set forth in SEQ ID NO:66 (or a variant of SEQ ID NO:66 with one or two amino acid modifications), and a CDR3 having the amino acid sequence set forth in SEQ ID NO:67 (or a variant of SEQ ID NO:67 with one or two amino acid modifications). An example of such a binder having these CDRs and the ability to bind to a PSMA polypeptide (e.g., a human PSMA polypeptide) includes, without limitation, the VH domain set forth in FIG. 8.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:65 (or a variant of SEQ ID NO:65 with one or two amino acid modifications), a CDR2 having the amino acid sequence set forth in SEQ ID NO:66 (or a variant of SEQ ID NO:66 with one or two amino acid modifications), and a CDR3 having the amino acid sequence set forth in SEQ ID NO:67 (or a variant of SEQ ID NO:67 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:68 (or a variant of SEQ ID NO:68 with one, two, three, four, five, six, seven, eight, nine, ten, or more amino acid modifications), a framework region 2 having the amino acid sequence set forth in SEQ ID NO:69 (or a variant of SEQ ID NO:69 with one, two, three, four, five, six, seven, eight, nine, ten, or more amino acid modifications), a framework region 3 having the amino acid sequence set forth in SEQ ID NO:70 (or a variant of SEQ ID NO:70 with one, two, three, four, five, six, seven, eight, nine, ten, or more amino acid modifications), and a framework region 4 having the amino acid sequence set forth in SEQ ID NO:71 (or a variant of SEQ ID NO:71 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 8 can be designed to include framework regions as set forth in FIG. 8 or can be designed to include one or more framework regions from another antibody or antibody fragment. For example, an antibody domain (e.g., a VH domain) can be designed to include the three CDRs set forth in FIG. 8 and the framework regions set forth in FIG. 8 except that framework region 1 having the amino acid set forth in SEQ ID NO:68 is replaced with a framework region 1 having the amino acid set forth in SEQ ID NO:36, a framework region 1 having the amino acid set forth in SEQ ID NO:52, or a framework region 1 having the amino acid set forth in SEQ ID NO:92. In another example, an Fab or scFv can be designed to include (a) the three CDRs set forth in FIG. 8, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:72. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:72. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:72.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:72, provided that the heavy chain variable domain includes the amino acid sequences set forth in SEQ ID NOs:65, 66, and 67. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:72, provided that the heavy chain variable domain includes the amino acid sequences set forth in SEQ ID NOs:65, 66, and 67.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:72 or the amino acid set forth in SEQ ID NO:72 with one, two, three, four, five, six, seven, eight, nine, or 10 amino acid modifications (e.g., amino acid substitutions, amino acid deletions, and/or amino acid additions). For example, antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:72 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:65, 66, and 67.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:65, (ii) a CDR2 that comprises, consists essentially of, or consists of the amino acid sequence set forth in SEQ ID NO:66, and (iii) a CDR3 that comprises, consists essentially of, or consists of the amino acid sequence set forth in SEQ ID NO:67. As used herein, a “CDR1 that consists essentially of the amino acid sequence set forth in SEQ ID NO:65” is a CDR1 that has zero, one, or two amino acid substitutions within SEQ ID NO:65, that has zero, one, two, three, four, or five amino acid residues directly preceding SEQ ID NO:65, and/or that has zero, one, two, three, four, or five amino acid residues directly following SEQ ID NO:65, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:65 include, without limitation, those set forth in Table 25.

TABLE 25
Exemplary CDR1s that consist essentially of the
amino acid sequence set forth in SEQ ID NO:65.

| Sequence | SEQ ID NO: |
|---|---|
| GFTFSSYW | 517 |
| GFTFDDYA | 518 |
| GFTFSDYY | 519 |
| GFTFSSYD | 520 |
| GFTFSNAW | 521 |
| GFTFSNSD | 522 |
| GFTFSNSD | 523 |
| GFTFDDYG | 524 |
| GFTFSSYS | 525 |
| GFTFSSYG | 526 |

As used herein, a “CDR2 that consists essentially of the amino acid sequence set forth in SEQ ID NO:66” is a CDR2 that has zero, one, or two amino acid substitutions within SEQ ID NO:66, that has zero, one, two, three, four, or five amino acid residues directly preceding SEQ ID NO:66, and/or that has zero, one, two, three, four, or five amino acid residues directly following SEQ ID NO:66, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:66 include, without limitation, those set forth in Table 26.

TABLE 26
Exemplary CDR2s that consist essentially of the
amino acid sequence set forth in SEQ ID NO: 66.

| Sequence | SEQ ID NO: |
|---|---|
| IKSKTDGGTT | 527 |
| INWNGGST | 528 |
| ISGSGGST | 529 |
| IGTAGDT | 530 |
| VNPNGGST | 531 |
| INSDGSST | 532 |
| IYSGGSST | 533 |
| IYHSGST | 534 |
| INPSGGST | 535 |
| INPNSGGT | 536 |

As used herein, a “CDR3 that consists essentially of the amino acid sequence set forth in SEQ ID NO:67” is a CDR3 that has zero, one, or two amino acid substitutions within SEQ TD NO:67, that has zero, one, two, three, four, or five amino acid residues directly preceding SEQ ID NO: 67, and/or that has zero, one, two, three, four, or five amino acid residues directly following SEQ ID NO:67, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO: 67 include, without limitation, those set forth in Table 27.

TABLE 27
Exemplary CDR3s that consist essentially of the
amino acid sequence set forth in SEQ ID NO: 67.

| Sequence | SEQ ID NO: |
|---|---|
| ARDGAD | 537 |
| AKNGAD | 538 |
| AKDGKD | 539 |
| AKDGND | 540 |
| ARDGND | 541 |
| ARDGKD | 542 |
| AKNGAD | 543 |
| AKDGSD | 544 |
| AKDGTD | 545 |
| ARDSAD | 546 |

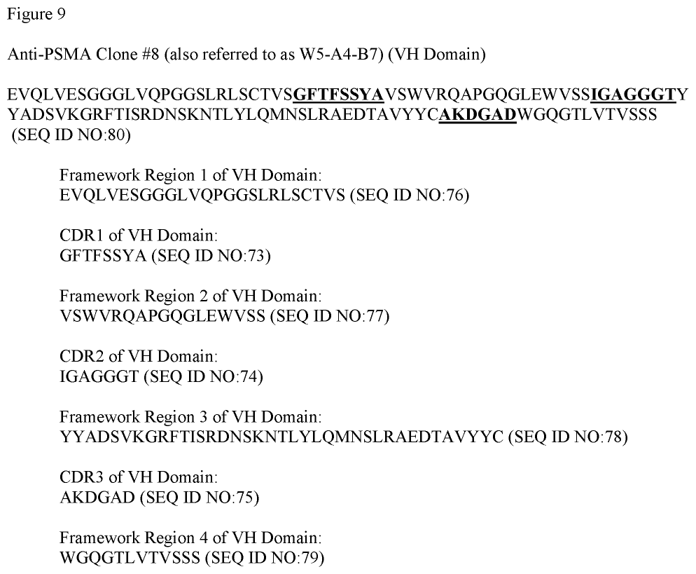

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:73 (or a variant of SEQ ID NO:73 with one or two amino acid modifications), a CDR2 having the amino acid sequence set forth in SEQ ID NO:74 (or a variant of SEQ ID NO:74 with one or two amino acid modifications), and a CDR3 having the amino acid sequence set forth in SEQ ID NO:75 (or a variant of SEQ ID NO:75 with one or two amino acid modifications). An example of such a binder having these CDRs and the ability to bind to a PSMA polypeptide (e.g., a human PSMA polypeptide) includes, without limitation, the VH domain set forth in FIG. 9.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:73 (or a variant of SEQ ID NO:73 with one or two amino acid modifications), a CDR2 having the amino acid sequence set forth in SEQ ID NO:74 (or a variant of SEQ ID NO:74 with one or two amino acid modifications), and a CDR3 having the amino acid sequence set forth in SEQ ID NO:75 (or a variant of SEQ ID NO:75 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:76 (or a variant of SEQ ID NO:76 with one, two, three, four, five, six, seven, eight, nine, ten, or more amino acid modifications), a framework region 2 having the amino acid sequence set forth in SEQ ID NO:77 (or a variant of SEQ ID NO:77 with one, two, three, four, five, six, seven, eight, nine, ten, or more amino acid modifications), a framework region 3 having the amino acid sequence set forth in SEQ ID NO:78 (or a variant of SEQ ID NO:78 with one, two, three, four, five, six, seven, eight, nine, ten, or more amino acid modifications), and a framework region 4 having the amino acid sequence set forth in SEQ ID NO:79 (or a variant of SEQ ID NO:79 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 9 can be designed to include framework regions as set forth in FIG. 9 or can be designed to include one or more framework regions from another antibody or antibody fragment. For example, an antibody domain (e.g., a VH domain) can be designed to include the three CDRs set forth in FIG. 9 and the framework regions set forth in FIG. 9 except that framework region 1 having the amino acid set forth in SEQ ID NO:76 is replaced with a framework region 1 having the amino acid set forth in SEQ ID NO:36, a framework region 1 having the amino acid set forth in SEQ ID NO:52, or a framework region 1 having the amino acid set forth in SEQ ID NO:92. In another example, an Fab or scFv can be designed to include (a) the three CDRs set forth in FIG. 9, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:80. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:80. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:80.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:80, provided that the heavy chain variable domain includes the amino acid sequences set forth in SEQ ID NOs:73, 74, and 75. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:80, provided that the heavy chain variable domain includes the amino acid sequences set forth in SEQ ID NOs:73, 74, and 75.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:80 or the amino acid set forth in SEQ ID NO:80 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:80 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:73, 74, and 75.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:73, (ii) a CDR2 that comprises, consists essentially of, or consists of the amino acid sequence set forth in SEQ ID NO:74, and (iii) a CDR3 that comprises, consists essentially of, or consists of the amino acid sequence set forth in SEQ ID NO:75. As used herein, a “CDR1 that consists essentially of the amino acid sequence set forth in SEQ ID NO:73” is a CDR1 that has zero, one, or two amino acid substitutions within SEQ ID NO:73, that has zero, one, two, three, four, or five amino acid residues directly preceding SEQ ID NO:73, and/or that has zero, one, two, three, four, or five amino acid residues directly following SEQ ID NO:73, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:73 include, without limitation, those set forth in Table 28.

TABLE 28
Exemplary CDR1s that consist essentially of the
amino acid sequence set forth in SEQ ID NO: 73.

| Sequence | SEQ ID NO: |
|---|---|
| GFTFSSYW | 547 |
| GFTFDDYA | 548 |
| GFTFSDYY | 549 |
| GFTFSSYD | 550 |
| GFTFSNAW | 551 |
| GFTFSNSD | 552 |
| GFTFSNSD | 553 |
| GFTFDDYG | 554 |
| GFTFSSYS | 555 |
| GFTFSSYG | 556 |

As used herein, a “CDR2 that consists essentially of the amino acid sequence set forth in SEQ ID NO:74” is a CDR2 that has zero, one, or two amino acid substitutions within SEQ ID NO:74, that has zero, one, two, three, four, or five amino acid residues directly preceding SEQ ID NO:74, and/or that has zero, one, two, three, four, or five amino acid residues directly following SEQ ID NO:74, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:74 include, without limitation, those set forth in Table 29.

TABLE 29
Exemplary CDR2s that consist essentially of the
amino acid sequence set forth in SEQ ID NO: 74.

| Sequence | SEQ ID NO: |
|---|---|
| IKSKTDGGTT | 557 |
| INWNGGST | 558 |
| ISGSGGST | 559 |
| IGTAGDT | 560 |
| VNPNGGST | 561 |
| INSDGSST | 562 |
| IYSGGSST | 563 |
| IYHSGST | 564 |
| INPSGGST | 565 |
| INPNSGGT | 566 |

As used herein, a “CDR3 that consists essentially of the amino acid sequence set forth in SEQ ID NO:75” is a CDR3 that has zero, one, or two amino acid substitutions within SEQ ID NO:75, that has zero, one, two, three, four, or five amino acid residues directly preceding SEQ ID NO:75, and/or that has zero, one, two, three, four, or five amino acid residues directly following SEQ ID NO:75, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:75 include, without limitation, those set forth in Table 30.

TABLE 30
Exemplary CDR3s that consist essentially of the
amino acid sequence set forth in SEQ ID NO: 75.

| Sequence | SEQ ID NO: |
|---|---|
| ARDGAD | 567 |
| AKNGAD | 568 |
| AKDGKD | 569 |
| AKDGND | 570 |
| ARDGND | 571 |
| ARDGKD | 572 |
| AKNGAD | 573 |
| AKDGSD | 574 |
| AKDGTD | 575 |
| ARDSAD | 576 |

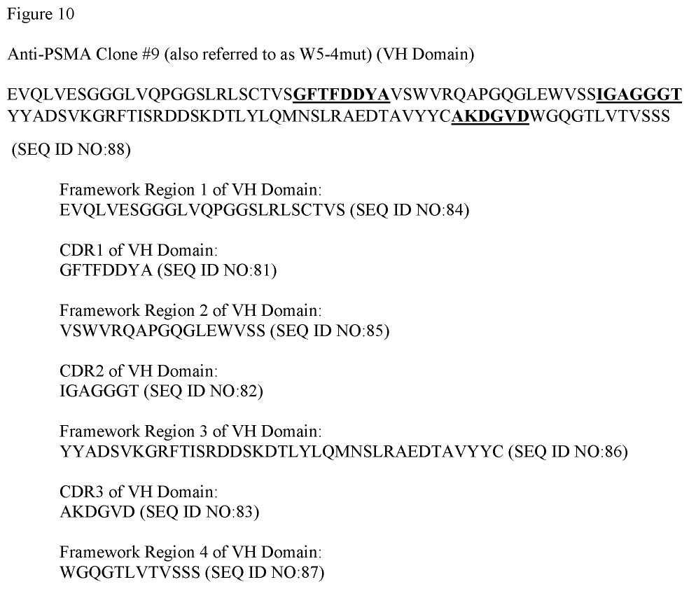

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:81 (or a variant of SEQ ID NO:81 with one or two amino acid modifications), a CDR2 having the amino acid sequence set forth in SEQ ID NO:82 (or a variant of SEQ ID NO:82 with one or two amino acid modifications), and a CDR3 having the amino acid sequence set forth in SEQ ID NO:83 (or a variant of SEQ ID NO:83 with one or two amino acid modifications). An example of such a binder having these CDRs and the ability to bind to a PSMA polypeptide (e.g., a human PSMA polypeptide) includes, without limitation, the VH domain set forth in FIG. 10.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:81 (or a variant of SEQ ID NO:81 with one or two amino acid modifications), a CDR2 having the amino acid sequence set forth in SEQ ID NO:82 (or a variant of SEQ ID NO:82 with one or two amino acid modifications), and a CDR3 having the amino acid sequence set forth in SEQ ID NO:83 (or a variant of SEQ ID NO:83 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:84 (or a variant of SEQ ID NO:84 with one, two, three, four, five, six, seven, eight, nine, ten, or more amino acid modifications), a framework region 2 having the amino acid sequence set forth in SEQ ID NO:85 (or a variant of SEQ ID NO:85 with one, two, three, four, five, six, seven, eight, nine, ten, or more amino acid modifications), a framework region 3 having the amino acid sequence set forth in SEQ ID NO:86 (or a variant of SEQ ID NO:86 with one, two, three, four, five, six, seven, eight, nine, ten, or more amino acid modifications), and a framework region 4 having the amino acid sequence set forth in SEQ ID NO:87 (or a variant of SEQ ID NO:87 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 10 can be designed to include framework regions as set forth in FIG. 10 or can be designed to include one or more framework regions from another antibody or antibody fragment. For example, an antibody domain (e.g., a VH domain) can be designed to include the three CDRs set forth in FIG. 10 and the framework regions set forth in FIG. 10 except that framework region 1 having the amino acid set forth in SEQ ID NO:84 is replaced with a framework region 1 having the amino acid set forth in SEQ ID NO:36, a framework region 1 having the amino acid set forth in SEQ ID NO:52, or a framework region 1 having the amino acid set forth in SEQ ID NO:92. In another example, an Fab or scFv can be designed to include (a) the three CDRs set forth in FIG. 10, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:88. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:88. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:88.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:88, provided that the heavy chain variable domain includes the amino acid sequences set forth in SEQ ID NOs:81, 82, and 83. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:88, provided that the heavy chain variable domain includes the amino acid sequences set forth in SEQ ID NOs:81, 82, and 83.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:88 or the amino acid set forth in SEQ ID NO:88 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:88 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:81, 82, and 83.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:81, (ii) a CDR2 that comprises, consists essentially of, or consists of the amino acid sequence set forth in SEQ ID NO:82, and (iii) a CDR3 that comprises, consists essentially of, or consists of the amino acid sequence set forth in SEQ ID NO:83. As used herein, a “CDR1 that consists essentially of the amino acid sequence set forth in SEQ ID NO:81” is a CDR1 that has zero, one, or two amino acid substitutions within SEQ ID NO:81, that has zero, one, two, three, four, or five amino acid residues directly preceding SEQ ID NO:81, and/or that has zero, one, two, three, four, or five amino acid residues directly following SEQ ID NO:81, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:81 include, without limitation, those set forth in Table 31.

TABLE 31
Exemplary CDR1s that consist essentially of the
amino acid sequence set forth in SEQ ID NO: 81.

| Sequence | SEQ ID NO: |
|---|---|
| GFTFSSYW | 577 |
| GFTFSSYA | 578 |
| GFTFSDYY | 579 |
| GFTFSSYD | 580 |
| GFTFSNAW | 581 |
| GFTFSNSD | 582 |
| GFTFSNSD | 583 |
| GFTFDDYG | 584 |
| GFTFSSYS | 585 |
| GFTFSSYG | 586 |

As used herein, a “CDR2 that consists essentially of the amino acid sequence set forth in SEQ ID NO:82” is a CDR2 that has zero, one, or two amino acid substitutions within SEQ ID NO:82, that has zero, one, two, three, four, or five amino acid residues directly preceding SEQ ID NO:82, and/or that has zero, one, two, three, four, or five amino acid residues directly following SEQ ID NO:82, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:82 include, without limitation, those set forth in Table 32.

TABLE 32
Exemplary CDR2s that consist essentially of the
amino acid sequence set forth in SEQ ID NO: 82.

| Sequence | SEQ ID NO: |
|---|---|
| IKSKTDGGTT | 587 |
| INWNGGST | 588 |
| ISGSGGST | 589 |
| IGTAGDT | 590 |
| VNPNGGST | 591 |
| INSDGSST | 592 |
| IYSGGSST | 593 |
| IYHSGST | 594 |
| INPSGGST | 595 |
| INPNSGGT | 596 |

As used herein, a “CDR3 that consists essentially of the amino acid sequence set forth in SEQ ID NO: 83” is a CDR3 that has zero, one, or two amino acid substitutions within SEQ TD NO:83, that has zero, one, two, three, four, or five amino acid residues directly preceding SEQ ID NO:83, and/or that has zero, one, two, three, four, or five amino acid residues directly following SEQ ID NO:83, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO: 83 include, without limitation, those set forth in Table 33.

TABLE 33
Exemplary CDR3s that consist essentially of the
amino acid sequence set forth in SEQ ID NO: 83.

| Sequence | SEQ ID NO: |
|---|---|
| ARDGAD | 597 |
| AKNGAD | 598 |
| AKDGKD | 599 |
| AKDGND | 600 |
| ARDGND | 601 |
| ARDGKD | 602 |
| AKNGAD | 603 |
| AKDGSD | 604 |
| AKDGTD | 605 |
| ARDSAD | 606 |

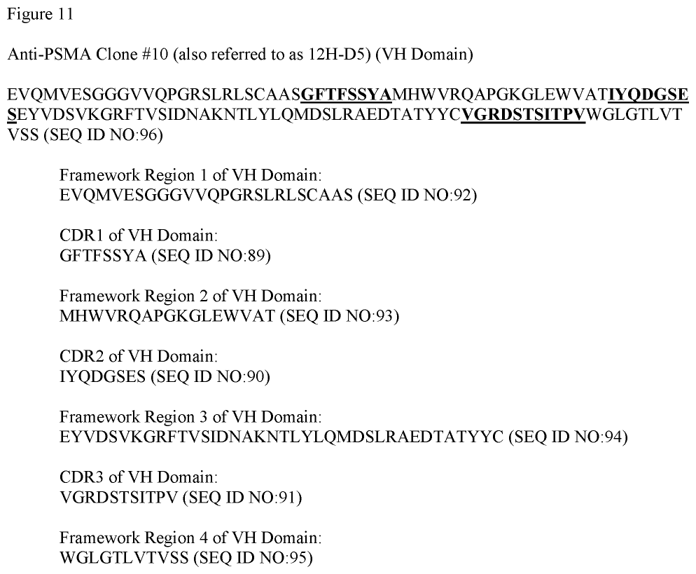

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:89 (or a variant of SEQ ID NO:89 with one or two amino acid modifications), a CDR2 having the amino acid sequence set forth in SEQ ID NO:90 (or a variant of SEQ ID NO:90 with one or two amino acid modifications), and a CDR3 having the amino acid sequence set forth in SEQ ID NO:91 (or a variant of SEQ ID NO:91 with one or two amino acid modifications). An example of such a binder having these CDRs and the ability to bind to a PSMA polypeptide (e.g., a human PSMA polypeptide) includes, without limitation, the VH domain set forth in FIG. 11.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:89 (or a variant of SEQ ID NO:89 with one or two amino acid modifications), a CDR2 having the amino acid sequence set forth in SEQ ID NO:90 (or a variant of SEQ ID NO:90 with one or two amino acid modifications), and a CDR3 having the amino acid sequence set forth in SEQ ID NO:91 (or a variant of SEQ ID NO:91 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:92 (or a variant of SEQ ID NO:92 with one, two, three, four, five, six, seven, eight, nine, ten, or more amino acid modifications), a framework region 2 having the amino acid sequence set forth in SEQ ID NO:93 (or a variant of SEQ ID NO:93 with one, two, three, four, five, six, seven, eight, nine, ten, or more amino acid modifications), a framework region 3 having the amino acid sequence set forth in SEQ ID NO:94 (or a variant of SEQ ID NO:94 with one, two, three, four, five, six, seven, eight, nine, ten, or more amino acid modifications), and a framework region 4 having the amino acid sequence set forth in SEQ ID NO:95 (or a variant of SEQ ID NO:95 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 11 can be designed to include framework regions as set forth in FIG. 11 or can be designed to include one or more framework regions from another antibody or antibody fragment. For example, an antibody domain (e.g., a VH domain) can be designed to include the three CDRs set forth in FIG. 11 and the framework regions set forth in FIG. 11 except that framework region 1 having the amino acid set forth in SEQ ID NO:92 is replaced with a framework region 1 having the amino acid set forth in SEQ ID NO:36, a framework region 1 having the amino acid set forth in SEQ ID NO:52, or a framework region 1 having the amino acid set forth in SEQ ID NO:60. In another example, an Fab or scFv can be designed to include (a) the three CDRs set forth in FIG. 11, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:96. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:96. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:96.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:96, provided that the heavy chain variable domain includes the amino acid sequences set forth in SEQ ID NOs:89, 90, and 91. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:96, provided that the heavy chain variable domain includes the amino acid sequences set forth in SEQ ID NOs:89, 90, and 91.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:96 or the amino acid set forth in SEQ ID NO:96 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:96 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:89, 90, and 91.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:89, (ii) a CDR2 that comprises, consists essentially of, or consists of the amino acid sequence set forth in SEQ ID NO:90, and (iii) a CDR3 that comprises, consists essentially of, or consists of the amino acid sequence set forth in SEQ ID NO:91. As used herein, a “CDR1 that consists essentially of the amino acid sequence set forth in SEQ ID NO:89” is a CDR1 that has zero, one, or two amino acid substitutions within SEQ ID NO:89, that has zero, one, two, three, four, or five amino acid residues directly preceding SEQ ID NO:89, and/or that has zero, one, two, three, four, or five amino acid residues directly following SEQ ID NO:89, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:89 include, without limitation, those set forth in Table 34.

TABLE 34
Exemplary CDR1s that consist essentially of the
amino acid sequence set forth in SEQ ID NO: 89.

| Sequence | SEQ ID NO: |
|---|---|
| GFTFSSYW | 607 |
| GFTFDDYA | 608 |
| GFTFSDYY | 609 |
| GFTFSSYD | 610 |
| GFTFSNAW | 611 |
| GFTFSNSD | 612 |
| GFTFSNSD | 613 |
| GFTFDDYG | 614 |
| GFTFSSYS | 615 |
| GFTFSSYG | 616 |

As used herein, a “CDR2 that consists essentially of the amino acid sequence set forth in SEQ ID NO:90” is a CDR2 that has zero, one, or two amino acid substitutions within SEQ ID NO:90, that has zero, one, two, three, four, or five amino acid residues directly preceding SEQ ID NO:90, and/or that has zero, one, two, three, four, or five amino acid residues directly following SEQ ID NO:90, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO:90 include, without limitation, those set forth in Table 35.

TABLE 35
Exemplary CDR2s that consist essentially of the
amino acid sequence set forth in SEQ ID NO: 90.

| Sequence | SEQ ID NO: |
|---|---|
| IKQDGSEK | 617 |
| ISWNSGSI | 618 |
| ISSSGSTI | 619 |
| IGTAGDT | 620 |
| IKSKTDGGTT | 621 |
| VSWNGSRT | 622 |
| INWNGGST | 623 |
| ISSSSSYI | 624 |
| ISGSGGST | 625 |
| IQCDGSQI | 626 |

As used herein, a “CDR3 that consists essentially of the amino acid sequence set forth in SEQ ID NO:91” is a CDR3 that has zero, one, or two amino acid substitutions within SEQ ID NO:91, that has zero, one, two, three, four, or five amino acid residues directly preceding SEQ ID NO:91, and/or that has zero, one, two, three, four, or five amino acid residues directly following SEQ ID NO:91, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO:91 include, without limitation, those set forth in Table 36.

TABLE 36
Exemplary CDR3s that consist essentially of the
amino acid sequence set forth in SEQ ID NO: 91.

| Sequence | SEQ ID NO: |
|---|---|
| VGRSSTSITPV | 627 |
| VGRNSTSITPV | 628 |
| VGRYSTSITPV | 629 |
| VGRQSTSITPV | 630 |
| VGRGSLSITPV | 631 |
| VGRGSCSITPV | 632 |
| VGRGSISITPV | 633 |
| VGRGSTSILPV | 634 |
| VGRGSTSLTPV | 635 |
| VGRGSNSITPV | 636 |

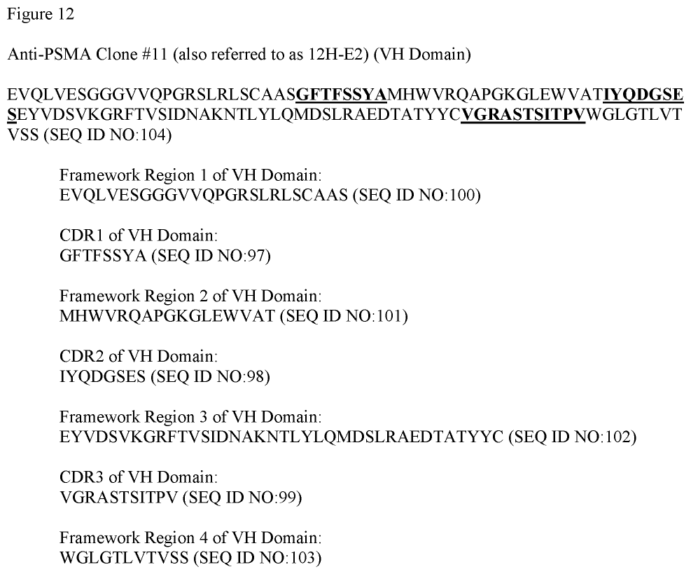

In another embodiment, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having a CDR1 having the amino acid sequence set forth in SEQ ID NO:97 (or a variant of SEQ ID NO:97 with one or two amino acid modifications), a CDR2 having the amino acid sequence set forth in SEQ ID NO:98 (or a variant of SEQ ID NO:98 with one or two amino acid modifications), and a CDR3 having the amino acid sequence set forth in SEQ ID NO:99 (or a variant of SEQ ID NO:99 with one or two amino acid modifications). An example of such a binder having these CDRs and the ability to bind to a PSMA polypeptide (e.g., a human PSMA polypeptide) includes, without limitation, the VH domain set forth in FIG. 12.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) and having a heavy chain variable domain having a CDR1 having the amino acid sequence set forth in SEQ ID NO:97 (or a variant of SEQ ID NO:97 with one or two amino acid modifications), a CDR2 having the amino acid sequence set forth in SEQ ID NO:98 (or a variant of SEQ ID NO:98 with one or two amino acid modifications), and a CDR3 having the amino acid sequence set forth in SEQ ID NO:99 (or a variant of SEQ ID NO:99 with one or two amino acid modifications) can include any appropriate framework regions. For example, such a binder (e.g., an antibody, antigen binding fragment, antibody domain, a CAR, a cell engager, and/or an ADC) can include a heavy chain variable domain that includes a framework region 1 having the amino acid sequence set forth in SEQ ID NO:100 (or a variant of SEQ ID NO:100 with one, two, three, four, five, six, seven, eight, nine, ten, or more amino acid modifications), a framework region 2 having the amino acid sequence set forth in SEQ ID NO:101 (or a variant of SEQ ID NO:101 with one, two, three, four, five, six, seven, eight, nine, ten, or more amino acid modifications), a framework region 3 having the amino acid sequence set forth in SEQ ID NO:102 (or a variant of SEQ ID NO:102 with one, two, three, four, five, six, seven, eight, nine, ten, or more amino acid modifications), and a framework region 4 having the amino acid sequence set forth in SEQ ID NO:103 (or a variant of SEQ ID NO:103 with one, two, three, four, five, six, seven, eight, nine, ten, or more amino acid modifications).

In some cases, a binder (e.g., an antibody, antigen binding fragment, antibody domain, a CAR, a cell engager, and/or an ADC) having any of the CDRs set forth in FIG. 12 can be designed to include framework regions as set forth in FIG. 12 or can be designed to include one or more framework regions from another antibody or antibody fragment. For example, an antibody domain (e.g., a VH domain) can be designed to include the three CDRs set forth in FIG. 12 and the framework regions set forth in FIG. 12 except that framework region 1 having the amino acid set forth in SEQ ID NO:100 is replaced with a framework region 1 having the amino acid set forth in SEQ ID NO:52, a framework region 1 having the amino acid set forth in SEQ ID NO:60, or a framework region 1 having the amino acid set forth in SEQ ID NO:92. In another example, an Fab or scFv can be designed to include (a) the three CDRs set forth in FIG. 12, (b) the framework regions set forth in FIG. 2A, 3A, 4-12, or 16A, 17A, 18A, 19A, 20A, or 21A, and (c) a light chain variable domain.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:104. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:104. In some cases, a binder (e.g., an antibody, antigen binding fragment, antibody domain, a CAR, a cell engager, and/or an ADC) provided herein can include (a) a heavy chain variable domain that includes an amino acid sequence having 100 percent identity to the amino acid sequence set forth in SEQ ID NO:104.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain that includes an amino acid sequence having at least 90 percent identity to the amino acid sequence set forth in SEQ ID NO:104, provided that the heavy chain variable domain includes the amino acid sequences set forth in SEQ ID NOs:97, 98, and 99. For example, a binder (e.g., an antibody, antigen binding fragment, antibody domain, a CAR, a cell engager, and/or an ADC) provided herein can include a heavy chain variable domain that includes an amino acid sequence having at least 90, 91, 92, 93, 94, 95, 96, 97, 98, or 99 percent identity to the amino acid sequence set forth in SEQ ID NO:104, provided that the heavy chain variable domain includes the amino acid sequences set forth in SEQ ID NOs:97, 98, and 99.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain having the amino acid sequence set forth in SEQ ID NO:104 or the amino acid set forth in SEQ ID NO:104 with one, two, three, four, five, six, seven, eight, nine, or 10 amino acid modifications (e.g., amino acid substitutions, amino acid deletions, and/or amino acid additions). For example, an antibody domain (e.g., a VH domain) provided herein can have the ability to bind to a PSMA polypeptide (e.g., a human PSMA polypeptide) and can include a heavy chain variable domain having the amino acid sequence set forth in SEQ ID NO:104 with one, two, three, four, five, six, seven, eight, nine, or 10 amino acid modifications (e.g., amino acid substitutions, amino acid deletions, and/or amino acid additions), provided that the heavy chain variable domain includes the amino acid sequences set forth in SEQ ID NOs:97, 98, and 99.

In some cases, a binder (e.g., an antibody, antigen binding fragment, antibody domain, a CAR, a cell engager, and/or an ADC) provided herein having the ability to bind to a PSMA polypeptide (e.g., a human PSMA polypeptide) can include a heavy chain variable domain comprising (i) a CDR1 that comprises, consists essentially of, or consists of the amino acid sequence set forth in SEQ ID NO:97, (ii) a CDR2 that comprises, consists essentially of, or consists of the amino acid sequence set forth in SEQ ID NO:98, and (iii) a CDR3 that comprises, consists essentially of, or consists of the amino acid sequence set forth in SEQ ID NO:99. As used herein, a “CDR1 that consists essentially of the amino acid sequence set forth in SEQ ID NO:97” is a CDR1 that has zero, one, or two amino acid substitutions within SEQ ID NO:97, that has zero, one, two, three, four, or five amino acid residues directly preceding SEQ ID NO:97, and/or that has zero, one, two, three, four, or five amino acid residues directly following SEQ ID NO:97, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR1 that consists essentially of the amino acid sequence set forth in SEQ ID NO:97 include, without limitation, those set forth in Table 37.

TABLE 37
Exemplary CDR1s that consist essentially of the
amino acid sequence set forth in SEQ ID NO: 97.

| Sequence | SEQ ID NO: |
|---|---|
| GFTFSSYW | 637 |
| GFTFDDYA | 638 |
| GFTFSDYY | 639 |
| GFTFSSYD | 640 |
| GFTFSNAW | 641 |
| GFTFSNSD | 642 |
| GFTFSNSD | 643 |
| GFTFDDYG | 644 |
| GFTFSSYS | 645 |
| GFTFSSYG | 646 |

As used herein, a “CDR2 that consists essentially of the amino acid sequence set forth in SEQ ID NO:98” is a CDR2 that has zero, one, or two amino acid substitutions within SEQ TD NO:98, that has zero, one, two, three, four, or five amino acid residues directly preceding SEQ ID NO: 98, and/or that has zero, one, two, three, four, or five amino acid residues directly following SEQ ID NO:98, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR2 that consists essentially of the amino acid sequence set forth in SEQ ID NO: 98 include, without limitation, those set forth in Table 38.

TABLE 38
Exemplary CDR2s that consist essentially of the
amino acid sequence set forth in SEQ ID NO: 98.

| Sequence | SEQ ID NO: |
|---|---|
| IKQDGSEK | 647 |
| ISWNSGSI | 648 |
| ISSSGSTI | 649 |
| IGTAGDT | 650 |
| IKSKTDGGTT | 651 |
| VSWNGSRT | 652 |
| INWNGGST | 653 |
| ISSSSSYI | 654 |
| ISGSGGST | 655 |
| IQCDGSQI | 656 |

As used herein, a “CDR3 that consists essentially of the amino acid sequence set forth in SEQ ID NO:99” is a CDR3 that has zero, one, or two amino acid substitutions within SEQ TD NO:99, that has zero, one, two, three, four, or five amino acid residues directly preceding SEQ ID NO:99, and/or that has zero, one, two, three, four, or five amino acid residues directly following SEQ ID NO:99, provided that the binder (e.g., an antibody, antigen binding fragment, antibody domain, a CAR, a cell engager, and/or an ADC) maintains its basic ability to bind to a PSMA polypeptide (e.g., a human PSMA polypeptide). Examples of a CDR3 that consists essentially of the amino acid sequence set forth in SEQ ID NO: 99 include, without limitation, those set forth in Table 39.

TABLE 39
Exemplary CDR3s that consist essentially of the
amino acid sequence set forth in SEQ ID NO: 99.

| Sequence | SEQ ID NO: |
|---|---|
| VGRSSTSITPV | 657 |
| VGRNSTSITPV | 658 |
| VGRYSTSITPV | 659 |
| VGRQSTSITPV | 660 |
| VGRGSLSITPV | 661 |
| VGRGSCSITPV | 662 |
| VGRGSISITPV | 663 |
| VGRGSTSILPV | 664 |
| VGRGSTSLTPV | 665 |
| VGRGSNSITPV | 666 |

When designing a single chain antibody (e.g., a scFv) having a heavy chain variable domain and a light chain variable domain, the two regions can be directly connected or can be connected using any appropriate linker sequence. For example, a heavy chain variable domain having the CDRs of SEQ ID NOs:1-3 or SEQ ID NOs:17-19 can be directly connected to a light chain variable domain having the CDRs of SEQ ID NOs:9-11 or SEQ ID NOs:25-27, respectively, via a linker sequence. Examples of linker sequences that can be used to connect a heavy chain variable domain and a light chain variable domain to create a scFv include, without limitation, those linkers set forth in FIG. 22.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations. In some cases, an amino acid modification can be made to improve the binding and/or contact with an antigen and/or to improve a functional activity of a binder (e.g., an antibody, antigen binding fragment, antibody domain, a CAR, a cell engager, and/or an ADC) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) a residue having a bulky side chain (e.g., phenylalanine) for glycine or other residue having a small side chain.

The percent sequence identity between a particular amino acid sequence (or a particular nucleic acid sequence) and an amino acid sequence (or a nucleic acid sequence) referenced by a particular sequence identification number is determined as follows. First, an amino acid sequence (or a nucleic acid sequence) is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q -l -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. A matched position refers to a position in which an identical nucleotide or amino acid residue occurs at the same position in aligned sequences. The percent sequence identity is determined by dividing the number of matches by the length of the sequence set forth in the identified sequence (e.g., SEQ ID NO:8, SEQ ID NO:16, SEQ ID NO:24, SEQ ID NO:32, SEQ ID NO:40, SEQ ID NO:48, SEQ ID NO:56, SEQ ID NO:64, SEQ ID NO:72, SEQ ID NO:80, SEQ ID NO:88, SEQ ID NO:96, or SEQ ID NO:104), followed by multiplying the resulting value by 100. For example, an amino acid sequence that has 100 matches when aligned with the sequence set forth in SEQ ID NO:104 is 84.7 percent identical to the sequence set forth in SEQ ID NO:104 (i.e., 100÷118×100=84.7). It is noted that the percent sequence identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr Opin. Biotechnol. 3: 348-354 (1992). Both naturally occurring and non-naturally occurring amino acids (e.g., artificially-derivatized amino acids) can be used to generate an amino acid sequence variant provided herein.

A representative number of binders (e.g., antibodies, antigen binding fragments, and/or antibody domains) having the ability to bind to a PSMA polypeptide (e.g., a human PSMA polypeptide) are further described in Table 40.

TABLE 40
Representative number of binders.

| | | SEQ ID NOs of | | | SEQ ID NOs of | |
|---|---|---|---|---|---|---|
| | SEQ ID NOs of | Heavy Chain | | SEQ ID NOs of | Light Chain | |
| | Heavy Chain | Variable | SEQ ID NO of | Light Chain | Variable | SEQ ID NO of |
| Clone # | Variable | Domain/Region | Heavy Chain | Variable | Domain/Region | Light Chain |
| (Antibody | Domain/Region | Framework | Variable | Domain/Region | Framework | Variable |
| type) | CDRs | Regions | Domain/Region | CDRs | Regions | Domain/Region |
| #1 (scFv) | 1, 2, 3 | 4, 5, 6, 7 | 8 | 9, 10, 11 | 12, 13, 14, 15 | 16 |
| #2 (scFv) | 17, 18, 19 | 20, 21, 22, 23 | 24 | 25, 26, 27 | 28, 29, 30, 31 | 32 |
| #3 (VH | 33, 34, 35 | 36, 37, 38, 39 | 40 | | | |
| domain) | | | | | | |
| #4 (VH | 41, 42, 43 | 44, 45, 46, 47 | 48 | | | |
| domain) | | | | | | |
| #5 (VH | 49, 50, 51 | 52, 53, 54, 55 | 56 | | | |
| domain) | | | | | | |
| #6 (VH | 57, 58, 59 | 60, 61, 62, 63 | 64 | | | |
| domain) | | | | | | |
| #7 (VH | 65, 66, 67 | 68, 69, 70, 71 | 72 | | | |
| domain) | | | | | | |
| #8 (VH | 73, 74, 75 | 76, 77, 78, 79 | 80 | | | |
| domain) | | | | | | |
| #9 (VH | 81, 82, 83 | 84, 85, 86, 87 | 88 | | | |
| domain) | | | | | | |
| #10 (VH | 89, 90, 91 | 92, 93, 94, 95 | 96 | | | |
| domain) | | | | | | |
| #11 (VH | 97, 98, 99 | 100, 101, 102, | 104 | | | |
| domain) | | 103 | | | | |

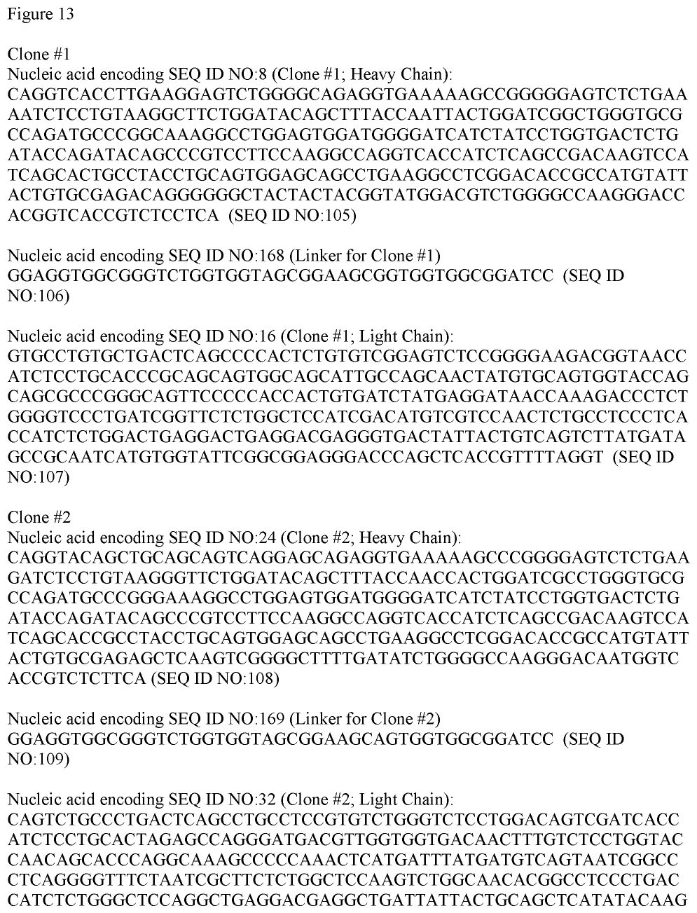

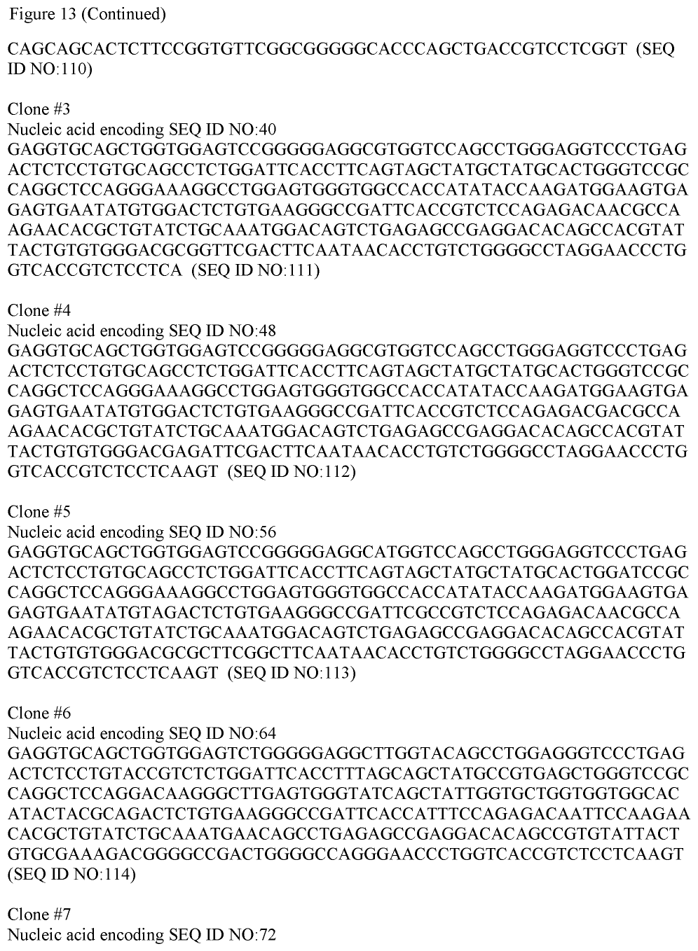

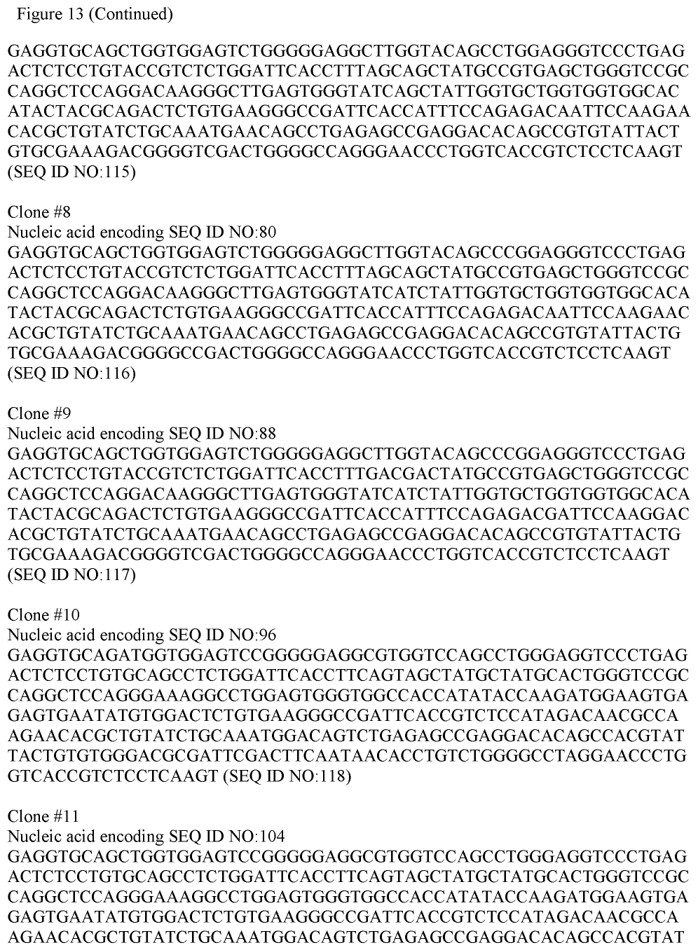

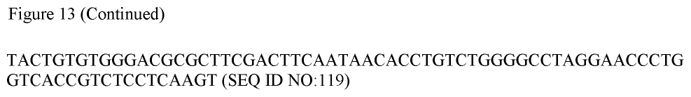

The binders (e.g., antibodies, antigen binding fragments, antibody domains, CARs, cell engagers, and/or ADCs) provided herein can be produced using any appropriate method. For example, the binders (e.g., antibodies, antigen binding fragments, antibody domains, CARs, and/or cell engagers) provided herein can be produced in recombinant host cells. For example, a nucleic acid encoding a binder (e.g., an antibody, antigen binding fragment, antibody domain, CAR, and/or cell engager) provided herein can be constructed, introduced into an expression vector, and expressed in suitable host cells. FIG. 13 is a sequence listing of nucleic acid sequences encoding exemplary binders (e.g., antibodies, antigen binding fragments, and/or antibody domains) described herein. In some cases, a binder (e.g., an antibody, antigen binding fragment, antibody domain, CAR, and/or cell engager) provided herein can be recombinantly produced in prokaryotic hosts such as E. coli, Bacillus brevis, Bacillus subtilis, Bacillus megaterium, Lactobacillus zeae casei, or Lactobacillus paracasei. A binder (e.g., an antibody, antigen binding fragment, antibody domain, CAR, and/or cell engager) provided herein also can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NS0 cells, baby hamster kidney (BHK) cells, or human embryonic kidney cell line HEK293). See, for example, the Frenzel et al. reference (Front Immunol., 4:217 (2013)).

In some cases, an antigen binding fragment or antibody domain provided herein can be produced by proteolytic digestion of an intact antibody. For example, an antigen binding fragment can be obtained by treating an antibody with an enzyme such as papain or pepsin. Papain digestion of whole antibodies can be used to produce F(ab)₂ or Fab fragments, while pepsin digestion of whole antibodies can be used to produce F(ab′)₂ or Fab′ fragments.

In some cases, a binder (e.g., an antibody, antigen binding fragment, antibody domain, CAR, cell engager, and/or ADC) provided herein can be substantially pure. The term “substantially pure” as used herein with reference to a binder (e.g., an antibody, antigen binding fragment, antibody domain, CAR, cell engager, and/or ADC) refers to the binder (e.g., an antibody, antigen binding fragment, antibody domain, CAR, cell engager, and/or ADC) as being substantially free of other polypeptides, lipids, carbohydrates, and nucleic acid with which it is naturally associated. Thus, a substantially pure binder (e.g., an antibody, antigen binding fragment, antibody domain, CAR, cell engager, and/or ADC) provided herein is any binder (e.g., an antibody, antigen binding fragment, antibody domain, CAR, cell engager, and/or ADC) that is removed from its natural environment and is at least 60 percent pure. A substantially pure binder (e.g., an antibody, antigen binding fragment, antibody domain, CAR, cell engager, and/or ADC) provided herein can be at least about 65, 70, 75, 80, 85, 90, 95, or 99 percent pure.

This document also provides bispecific binders (e.g., bispecific antibodies, bispecific antigen binding fragments, and/or bispecific antibody domains) that bind to two different epitopes with at least one being an epitope of a PSMA polypeptide (e.g., a human PSMA polypeptide). In some cases, a bispecific binder provided herein can be designed to bind to two different epitopes of the same PSMA polypeptide (e.g., a human PSMA polypeptide). In some cases, a bispecific binder provided herein can bind to a PSMA polypeptide (e.g., a human PSMA polypeptide) and to an epitope on a different polypeptide (e.g., a CD3 polypeptide). Bispecific binders can be produced by chemically conjugating two different binders (e.g., antibodies, antigen binding fragments, and/or antibody domains) together. Bispecific binders also can be produced by fusing two antibody-producing cells, e.g., hybridomas, to make a hybrid cell line that produces two different heavy and two different light chains within the same cell, which can result in, for example, bispecific IgG molecules. See, Brinkmann and Kontermann, MAbs., 9(2):182-212 (2017).

In some cases, a binder (e.g., an antibody, antigen binding fragment, antibody domain, CAR, and/or cell engager) provided herein can be fused or conjugated (e.g., covalently or non-covalently attached) to another polypeptide or other moiety to provide a fusion protein or conjugate. For example, a binder (e.g., an antibody, antigen binding fragment, antibody domain, CAR, and/or cell engager) provided herein can be conjugated (e.g., covalently or non-covalently attached) to a polymer (e.g., polyethylene glycol (PEG), polyethylenimine (PEI) modified with PEG (PEI-PEG), and/or polyglutamic acid (PGA) (N-(2-Hydroxypropyl) methacrylamide (HPMA) copolymers), hyaluronic acid, a fluorescent substance, a luminescent substance, a hapten, an enzyme, a metal chelate, a drug, a radioisotope, and/or a cytotoxic agent. Any appropriate method can be used to conjugate (e.g., covalently or non-covalently attach) another polypeptide or other moiety to a binder (e.g., an antibody, antigen binding fragment, antibody domain, CAR, and/or cell engager) provided herein. For example, another polypeptide or other moiety can be conjugated to a binder (e.g., an antibody, antigen binding fragment, antibody domain, CAR, and/or cell engager) provided herein using the methods described in U.S. Pat. No. 8,021,661.

In some cases, a binder (e.g., an antibody, antigen binding fragment, antibody domain, CAR, cell engager, and/or ADC) provided herein can be modified with a moiety that improves its stabilization and/or retention in circulation, for example, in blood, serum, or other tissues by, for example, at least 1.5-, 2-, 5-, 10-, or 50-fold. For example, a binder (e.g., an antibody, antigen binding fragment, antibody domain, CAR, cell engager, and/or ADC) provided herein can be attached (e.g., covalently or non-covalently attached) to a polymer such as a substantially non-antigenic polymer. Examples of substantially non-antigenic polymers that can be used as described herein include, without limitation, polyalkylene oxides and polyethylene oxides. In some cases, a polymer used herein can have any appropriate molecule weight. For example, a polymer having an average molecular weight from about 200 Daltons to about 35,000 Daltons (e.g., from about 1,000 to about 15,000 Daltons or from about 2,000 to about 12,500 Daltons) can be used. In some cases, a binder (e.g., an antibody, antigen binding fragment, antibody domain, CAR, cell engager, and/or ADC) provided herein can be attached (e.g., covalently or non-covalently) to a water soluble polymer. Examples of water soluble polymers that can be used as described herein include, without limitation, hydrophilic polyvinyl polymers, polyvinylalcohol, polyvinylpyrrolidone, polyalkylene oxide homopolymers, polyethylene glycol (PEG), polypropylene glycols, polyoxyethylenated polyols, and copolymers thereof and/or block copolymers thereof provided that the water solubility of the copolymer or block copolymers is maintained.

In some cases, a binder (e.g., an antibody, antigen binding fragment, antibody domain, CAR, cell engager, and/or ADC) provided herein can be attached (e.g., covalently or non-covalently attached) to one or more polyoxyalkylenes (e.g., polyoxyethylene, polyoxypropylene, or block copolymers of polyoxyethylene and polyoxypropylene), polymethacrylates, carbomers, branched or unbranched polysaccharides, or combinations thereof. For example, a binder (e.g., an antibody, antigen binding fragment, antibody domain, CAR, cell engager, and/or ADC) provided herein can be covalently attached to polyoxyethylene.

This document also provides ADCs. The term “ADC” as used herein refers to a conjugate that includes (a) an antigen binding domain and (b) at least one drug covalently linked directly or indirectly to that antigen binding domain. In some cases, an ADC described herein can include (a) an antigen binding domain having the ability to bind to a PSMA polypeptide (e.g., a human PSMA polypeptide) and (b) at least one drug covalently linked directly or indirectly to that antigen binding domain. Any appropriate binder (e.g., an antibody, antigen binding fragment, and/or antibody domain) provided herein and having the ability to bind to a PSMA polypeptide (e.g., a human PSMA polypeptide) can be used as an antigen binding domain to make an ADC described herein. For example, any of the binders set forth in Table 40 can be used to make an ADC having the ability to bind to a PSMA polypeptide (e.g., a human PSMA polypeptide). Examples of drugs that can be used to make an ADC described herein include, without limitation, auristatins (e.g., monomethyl auristatin E (MMAE)), mertansine (DM-1), and pyrrolobenzodiazepine (PBD) dimers. Any appropriate ADC linker can be used to covalently attach one or more drugs to an antigen binding domain having the ability to bind to a PSMA polypeptide (e.g., a human PSMA polypeptide) to form an ADC provided herein. For example, cleavable or non-cleavable ADC linkers can be used to covalently attach one or more drugs to an antigen binding domain having the ability to bind to a PSMA polypeptide (e.g., a human PSMA polypeptide) to form an ADC provided herein. Examples of ADC linkers can be used to covalently attach one or more drugs to an antigen binding domain having the ability to bind to a PSMA polypeptide (e.g., a human PSMA polypeptide) to form an ADC provided herein include, without limitation, ADC disulfide linkers, ADC hydrazone linkers, ADC peptide linkers, ADC thioether linkers, and ADC PEG-containing linkers.

This document also provides nucleic acid molecules (e.g., isolated nucleic acid molecules) having a nucleic acid sequence encoding at least part of a binder (e.g., an antibody, antigen binding fragment, antibody domain, CAR, and/or cell engager) provided herein. For example, an isolated nucleic acid molecule provided herein can include a nucleic acid sequence encoding a heavy chain variable domain such as a heavy chain variable domain as set forth in FIG. 2A or 3A. In another example, an isolated nucleic acid molecule provided herein can include a nucleic acid sequence encoding a light chain variable domain such as a light chain variable domain as set forth in FIG. 2B or 3B. In some cases, an isolated nucleic acid molecule provided herein can include a nucleic acid sequence encoding both (a) a heavy chain variable domain and (b) a light chain variable domain, with or without, encoding a linker polypeptide set forth in FIG. 22. A nucleic acid provided herein (e.g., an isolated nucleic acid molecule) can be single stranded or double stranded nucleic acid of any appropriate type (e.g., DNA, RNA, or DNA/RNA hybrids).

This document also provides vectors (e.g., plasmid vectors or viral vectors) containing one or more nucleic acids provided herein. An example of a plasmid vector that can be designed to include one or more nucleic acids having a nucleic acid sequence encoding at least part of a binder (e.g., an antibody, antigen binding fragment, antibody domain, CAR, and/or cell engager) provided herein includes, without limitation, phagemids. Examples of viral vectors that can be designed to include one or more nucleic acids having a nucleic acid sequence encoding at least part of a binder (e.g., an antibody, antigen binding fragment, antibody domain, CAR, and/or cell engager) provided herein include, without limitation, retroviral vectors, parvovirus-based vectors (e.g., adenoviral-based vectors and adeno-associated virus (AAV)-based vectors), lentiviral vectors (e.g., herpes simplex (HSV)-based vectors), poxviral vectors (e.g., vaccinia virus-based vectors and fowlpox virus-based vectors), and hybrid or chimeric viral vectors. For example, a viral vector having an adenoviral backbone with lentiviral components such as those described elsewhere (Zheng et al., Nat. Biotech., 18(2): 176-80 (2000); WO 98/22143; WO 98/46778; and WO 00/17376) or viral vectors having an adenoviral backbone with AAV components such as those described elsewhere (Fisher et al., Hum. Gene Ther., 7:2079-2087 (1996)) can be designed to include one or more nucleic acids having a nucleic acid sequence encoding at least part of a binder (e.g., an antibody, antigen binding fragment, antibody domain, CAR, and/or cell engager) provided herein.

In some cases, a vector (e.g., a plasmid vector or a viral vector) provided herein can include a nucleic acid sequence encoding scFv or antibody domain (e.g., a VH domain) provided herein. In some cases, a vector (e.g., a plasmid vector or a viral vector) provided herein can include a nucleic acid sequence encoding CAR provided herein. In some cases, a vector (e.g., a plasmid vector or a viral vector) provided herein can include a nucleic acid sequence encoding cell engager provided herein.

A vector provided herein (e.g., a plasmid vector or viral vector provided herein) can include any appropriate promoter and other regulatory sequence (e.g., transcription and translation initiation and termination codons) operably linked the nucleic acid sequence encoding at least part of a binder (e.g., an antibody, antigen binding fragment, antibody domain, CAR, and/or cell engager) provided herein. In some cases, a promoter used to drive expression can be a constitutive promotor or a regulatable promotor. Examples of regulatable promoters that can be used as described herein include, without limitation, inducible promotors, repressible promotors, and tissue-specific promoters. Examples of viral promotors that can be used as described herein include, without limitation, adenoviral promotors, vaccinia virus promotors, CMV promotors (e.g., immediate early CMV promotors), and AAV promoters.

Any appropriate method can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein. For example, molecule cloning techniques can be used to make a nucleic acid molecule (or vector such as a plasmid vector or viral vector) having a nucleic acid sequence encoding at least part of a binder (e.g., an antibody, antigen binding fragment, antibody domain, CAR, and/or cell engager) provided herein as described elsewhere (see, e.g., Sambrook et al., Molecular Cloning: A Laboratory Manual, 2nd edition, Cold Spring Harbor Laboratory, NY (1989); and Ausubel et al., Current Protocols in Molecular Biology, Green Publishing Associates and John Wiley & Sons, New York, N.Y. (1994)).

This document also provides host cells that include a nucleic acid provided herein (e.g., a nucleic acid having a nucleic acid sequence encoding at least part of a binder (e.g., an antibody, antigen binding fragment, antibody domain, CAR, and/or cell engager) provided herein). Host cells that can be designed to include one or more nucleic acids provided herein can be prokaryotic cells or eukaryotic cells. Examples of prokaryotic cells that can be designed to include a nucleic acid provided herein include, without limitation, E. coli (e.g., Tb-1, TG-1, DH5a, XL-Blue MRF (Stratagene), SA2821, or Y1090 cells), Bacillus subtilis, Salmonella typhimurium, Serratia marcescens, or Pseudomonas (e.g., P. aerugenosa) cells. Examples of eukaryotic cells that can be designed to include a nucleic acid provided herein include, without limitation, insect cells (e.g., Sf9 or Ea4 cells), yeast cells (e.g., S. cerevisiae cells), and mammalian cells (e.g., mouse, rat, hamster, monkey, or human cells). For example, VERO cells, HeLa cells, 3T3 cells, chinese hamster ovary (CHO) cells, W138 BHK cells, COS-7 cells, and MDCK cells can be designed to include a nucleic acid provided herein. Any appropriate method can be used to introduce one or more nucleic acids provided herein (e.g., a vector such as a plasmid vector or viral vector having a nucleic acid sequence encoding at least part of a binder provided herein) into a host cell. For example, calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, electroporation, or combinations thereof can be used to introduce a nucleic acid provided herein into a host cell (see, e.g., Sambrook et al., Molecular Biology: A Laboratory Manual, Cold Spring Harbor Laboratory, NY (1989); Davis et al., Basic Methods in Molecular Biology (1986); and Neumann et al., EMBO J., 1:841 (1982)).

In some cases, cells such as T cells, stem cells (e.g., induced pluripotent stem cells or mesenchymal stem cells), or NK cells can be designed to express one or more nucleic acids encoding a CAR described herein. For example, a population of T cells can be infected with viral vectors designed to express nucleic acid encoding a CAR described herein (e.g., a CAR having the ability to bind to a PSMA polypeptide).

In some cases, cells such as T cells, stem cells (e.g., induced pluripotent stem cells or mesenchymal stem cells), or NK cells can be designed to express one or more nucleic acids encoding a cell engager described herein. For example, a population of T cells can be infected with viral vectors designed to express nucleic acid encoding a cell engager described herein (e.g., a cell engager having the ability to bind to a PSMA polypeptide).

In some cases, a binder (e.g., an antibody, antigen binding fragment, antibody domain, CAR, and/or cell engager) provided herein can be produced using a method that includes (a) introducing nucleic acid encoding the polypeptide into a host cell; (b) culturing the host cell in culture medium under conditions sufficient to express the polypeptide; (c) harvesting the polypeptide from the cell or culture medium; and (d) purifying the polypeptide (e.g., to reach at least 50, 60, 70, 80, 90, 95, 97, 98, or 99 percent purity).

In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) having cancer to treat that mammal. In some cases, a binder (e.g., an antibody, antigen binding fragment, antibody domain, cell engager, and/or ADC) provided herein, a nucleic acid provided herein (e.g., nucleic acid encoding an antibody, antigen binding fragment, antibody domain, CAR, and/or cell engager provided herein), a vector provided herein (e.g., a viral vector designed to express an antibody, antigen binding fragment, antibody domain, CAR, and/or cell engager provided herein), and/or a host cell provided herein (e.g., a host cell designed to express an antibody, antigen binding fragment, antibody domain, CAR, and/or cell engager provided herein) can be formulated as a pharmaceutical composition for administration to a mammal (e.g. a human) to reduce the number of cancer cells within the mammal and/or to increase the survival of the mammal suffering from cancer. For example, a binder (e.g., an antibody, antigen binding fragment, antibody domain, cell engager, and/or ADC) provided herein having the ability to bind to a PSMA polypeptide (e.g., a human PSMA polypeptide) can be formulated as a pharmaceutical composition for administration to a mammal (e.g. a human). In some cases, a pharmaceutical composition provided herein can include a pharmaceutically acceptable carrier such as a buffer, a salt, a surfactant, a sugar, a tonicity modifier, or combinations thereof as, for example, described elsewhere (Gervasi, et al., Eur. J. Pharmaceutics and Biopharmaceutics, 131:8-24 (2018)). Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), or combinations thereof. For example, a pharmaceutical composition designed to include a binder (e.g., an antibody, antigen binding fragment, antibody domain, CAR, cell engager, and/or ADC) provided herein (or a nucleic acid, a vector, or a host cell provided herein) can be formulated to include a buffer (e.g., an acetate, citrate, histidine, succinate, phosphate, or hydroxymethylaminomethane (Tris) buffer), a surfactant (e.g., polysorbate 20, polysorbate 80, or poloxamer 188), and a sugar such as sucrose. Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, or combinations thereof. For example, a pharmaceutical composition provided herein can be formulated to include one or more binders (e.g., one or more antibodies, one or more antigen binding fragments, one or more antibody domains, one or more cells designed to express a CAR having the ability to bind to a PSMA polypeptide, one or more cell engagers, and/or one or more ADCs) provided herein in combination with one or more checkpoint inhibitors such as anti-PD-1 antibodies or PD-1 inhibitors (e.g., cemiplimab, nivolumab, pembrolizumab, JTX-4014, spartalizumab, camrelizumab, sintilimab, tislelizumab, toripalimab, dostarlimab, INCMGA00012, AMP-224, or AMP-514), anti-PD-L1 antibodies or PD-L1 inhibitors (e.g., avelumab, durvalumab, atezolizumab, KN035, CK-301, AUNP12, CA-170, or BMS-986189), and/or anti-CTLA-4 antibodies (e.g., ipilimumab).

In some cases, when a pharmaceutical composition is formulated to include one or more binders (e.g., one or more antibodies, one or more antigen binding fragments, one or more antibody domains, one or more cells designed to express a CAR having the ability to bind to a PSMA polypeptide, one or more cell engagers, and/or one or more ADCs) provided herein, any appropriate concentration of the binder can be used. For example, a pharmaceutical composition provided herein can be formulated to be a liquid that includes from about 1 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, CAR⁺ cell population, cell engager, and/or ADC) provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a binder (e.g., an antibody, antigen binding fragment, antibody domain, cell engager, and/or ADC) provided herein. In some cases, a pharmaceutical composition containing a binder (e.g., an antibody, antigen binding fragment, and/or antibody domain) provided herein can be formulated as a dosage form with a titer of the binder being from about 1×10⁵ to about 1×10¹² (e.g., from about 1×10⁵ to about 1×10¹⁰, from about 1×10⁵ to about 1×10⁸, from about 1×10⁶ to about 1×10¹², from about 1×10⁶ to about 1×10¹², from about 1×10⁸ to about 1×10¹², from about 1×10⁹ to about 1×10¹², from about 1×10⁶ to about 1×10¹¹, or from about 1×10⁷ to about 1×10¹⁰).

In some cases, when a pharmaceutical composition is formulated to include one or more nucleic acids (e.g., vectors such as viral vectors) encoding at least part of a binder (e.g., an antibody, antigen binding fragment, antibody domain, CAR, and/or cell engager) provided herein, any appropriate concentration of the nucleic acid can be used. For example, a pharmaceutical composition provided herein can be formulated to be a liquid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 2 mg to about 200 mg, from about 10 mg to about 300 mg, from about 25 mg to about 300 mg, from about 50 mg to about 150 mg, or from about 150 mg to about 300 mg) of a nucleic acid provided herein per mL. In another example, a pharmaceutical composition provided herein can be formulated to be a solid or semi-solid that includes from about 0.5 mg to about 500 mg (e.g., from about 1 mg to about 500 mg, from about 10 mg to about 500 mg, from about 50 mg to about 500 mg, from about 100 mg to about 500 mg, from about 0.5 mg to about 250 mg, from about 0.5 mg to about 150 mg, from about 0.5 mg to about 100 mg, from about 0.5 mg to about 50 mg, from about 1 mg to about 300 mg, from about 10 mg to about 300 mg, from about 25 mg to about 300 mg, from about 50 mg to about 150 mg, or from about 150 mg to about 300 mg) of a nucleic acid provided herein.

In some cases, a pharmaceutical composition designed to include a binder (e.g., an antibody, antigen binding fragment, antibody domain, cell engager, and/or ADC) provided herein can be formulated to include one or more agents capable of reducing aggregation of the binder when formulated. Examples of such agents that can be used as described herein include, without limitation, methionine, arginine, lysine, aspartic acid, glycine, glutamic acid, and combinations thereof. In some cases, one or more of these amino acids can be included within the formulation at a concentration from about 0.5 mM to about 145 mM (e.g., from about 1 mM to about 145 mM, from about 10 mM to about 145 mM, from about 100 mM to about 145 mM, from about 0.5 mM to about 125 mM, from about 0.5 mM to about 100 mM, from about 0.5 mM to about 75 mM, or from about 10 mM to about 100 mM).

A pharmaceutical composition provided herein can be in any appropriate form. For example, a pharmaceutical composition provided herein can designed to be a liquid, a semi-solid, or a solid. In some cases, a pharmaceutical composition provided herein can be a liquid solution (e.g., an injectable and/or infusible solution), a dispersion, a suspension, a tablet, a pill, a powder, a microemulsion, a liposome, or a suppository. In some cases, a pharmaceutical composition provided herein can be lyophilized. In some cases, a pharmaceutical composition provided herein (e.g., a pharmaceutical composition that includes one or more binders (e.g., one or more antibodies, one or more antigen binding fragments, one or more antibody domains, one or more cell engagers, and/or one or more ADCs) provided herein can be formulated with a carrier or coating designed to protect against rapid release. For example, a pharmaceutical composition provided herein can be formulated as a controlled release formulation or as a regulated release formulation as described elsewhere (U.S. Patent Application Publication Nos. 2019/0241667; 2019/0233522; and 2019/0233498).

This document also provides methods for administering a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) to a mammal (e.g., a human). For example, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g., a human) having cancer to treat that mammal. In some cases, a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, and/or host cell (e.g., CAR⁺ cells) provided herein) can be administered to a mammal (e.g. a human) to reduce the number of cancer cells within the mammal and/or to increase the survival of the mammal suffering from cancer.

Any appropriate cancer can be treated using a composition (e.g., a pharmaceutical composition provided herein) containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein). For example, a mammal (e.g., a human) having cancer can be treated by administering a composition (e.g., a pharmaceutical composition) containing one or more binders (e.g., one or more antibodies, one or more antigen binding fragments, one or more antibody domains, one or more cell engagers, and/or one or more ADCs) provided herein to that mammal. Examples of cancers that can be treated as described herein include, without limitation, cancers with PSMA expressed by cancer cells, stromal cells, and/or cells of a tumor neovasculature. Examples of such cancers include, without limitation, lung cancers, prostate cancers, colorectal cancers, renal cell carcinomas, carcinomas of the bladder, primary brain tumors, shwannomas, thyroid carcinomas, squamous cell carcinomas (SCCs), adrenocortical carcinomas, gastric carcinomas, pancreatic carcinomas, gynecologic malignancies, high grade sarcomas, and breast cancers. In some cases, a mammal (e.g., a human) having a PSMA⁺ cancer (e.g., a PSMA⁺ lung cancer, a PSMA⁺ prostate cancer, a PSMA⁺ brain cancer, a PSMA⁺ renal cancer, a PSMA⁺ colorectal cancer, a PSMA⁺ gastric cancer, a PSMA⁺ gynecologic cancer, or a PSMA⁺ SCC) can be administered a composition (e.g., a pharmaceutical composition) containing one or more binders (e.g., one or more antibodies, one or more antigen binding fragments, one or more antibody domains, one or more cell engagers, and/or one or more ADCs) provided herein to treat that mammal (e.g., to reduce the number of cancer cells within the mammal).

Any appropriate method can be used to administer a composition (e.g., a pharmaceutical composition) provided herein to a mammal (e.g., a human). For example, a composition provided herein (e.g., a pharmaceutical composition containing one or more binders provided herein such as one or more antibodies, one or more antigen binding fragments, one or more antibody domains, one or more cell engagers, and/or one or more ADCs provided herein) can be administered to a mammal (e.g., a human) intravenously (e.g., via an intravenous injection or infusion), subcutaneously (e.g., via a subcutaneous injection), intraperitoneally (e.g., via an intraperitoneal injection), orally, via inhalation, or intramuscularly (e.g., via intramuscular injection). In some cases, the route and/or mode of administration of a composition (e.g., a pharmaceutical composition provided herein) can be adjusted for the mammal being treated.

In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that reduces the number of cancer cells within a mammal having cancer without producing significant toxicity to the mammal. In some cases, an effective amount of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be an amount that increases the survival time of a mammal having cancer as compared to a control mammal having comparable cancer and not treated with the composition. For example, an effective amount of a binder (e.g., an antibody, antigen binding fragment, antibody domain, cell engager, and/or ADC) provided herein can be from about 0.001 mg/kg to about 100 mg/kg (e.g., from about 0.001 mg/kg to about 90 mg/kg, from about 0.001 mg/kg to about 80 mg/kg, from about 0.001 mg/kg to about 70 mg/kg, from about 0.001 mg/kg to about 60 mg/kg, from about 0.001 mg/kg to about 50 mg/kg, from about 0.001 mg/kg to about 40 mg/kg, from about 0.001 mg/kg to about 30 mg/kg, from about 0.005 mg/kg to about 100 mg/kg, from about 0.01 mg/kg to about 100 mg/kg, from about 0.05 mg/kg to about 100 mg/kg, from about 0.1 mg/kg to about 100 mg/kg, from about 0.5 mg/kg to about 100 mg/kg, from about 1 mg/kg to about 100 mg/kg, from about 5 mg/kg to about 100 mg/kg, from about 0.01 mg/kg to about 25 mg/kg, from about 0.1 mg/kg to about 30 mg/kg, from about 0.15 mg/kg to about 25 mg/kg, from about 0.2 mg/kg to about 20 mg/kg, from about 0.5 mg/kg to about 20 mg/kg, from about 1 mg/kg to about 30 mg/kg, from about 1 mg/kg to about 25 mg/kg, from about 1 mg/kg to about 20 mg/kg, from about 2 mg/kg to about 20 mg/kg, from about 5 mg/kg to about 30 mg/kg, from about 10 mg/kg to about 30 mg/kg, from about 15 mg/kg to about 30 mg/kg, from about 20 mg/kg to about 30 mg/kg, from about 3 mg/kg to about 30 mg/kg, from about 0.5 mg/kg to about 10 mg/kg, from about 1 mg/kg to about 10 mg/kg, from about 1 mg/kg to about 5 mg/kg, or from about 1 mg/kg to about 3 mg/kg). The effective amount can remain constant or can be adjusted as a sliding scale or variable dose depending on the mammal's response to treatment. Various factors can influence the actual effective amount used for a particular application. For example, the severity of cancer when treating a mammal having cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective amount of a composition provided herein (e.g., a pharmaceutical composition containing one or more binders provided herein) that is administered.

In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that reduces the number of cancer cells within a mammal having cancer without producing significant toxicity to the mammal. In some cases, an effective frequency of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a frequency that increases the survival time of a mammal having cancer as compared to a control mammal having comparable cancer and not treated with the composition. For example, an effective frequency of administration of a pharmaceutical composition provided herein such as a pharmaceutical composition containing one or more binders provided herein can be from about twice daily to about once a year (e.g., from about twice daily to about once a month, from about twice daily to about once a week, from about once daily to about once a month, or from one once daily to about once a week). In some cases, the frequency of administration of a pharmaceutical composition provided herein such as a pharmaceutical composition containing one or more binders provided herein can be daily. The frequency of administration of a pharmaceutical composition provided herein such as a pharmaceutical composition containing one or more binders provided herein can remain constant or can be variable during the duration of treatment. Various factors can influence the actual effective frequency used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective frequency of administration of a composition provided herein (e.g., a pharmaceutical composition containing one or more binders provided herein).

In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that reduces the number of cancer cells within a mammal without producing significant toxicity to the mammal. In some cases, an effective duration of administration of a composition containing one or more binders (e.g., one or more antibodies, one or more antigen binding fragments, one or more antibody domains, one or more cell engagers, and/or one or more ADCs) provided herein (or a nucleic acid, vector, or host cell (e.g., CAR⁺ cells) provided herein) (e.g., a pharmaceutical composition provided herein) can be a duration that increases the survival time of a mammal having cancer as compared to a control mammal having comparable cancer and not treated with the composition. For example, an effective duration of administration of a pharmaceutical composition provided herein such as a pharmaceutical composition containing one or more binders provided herein can vary from a single time point of administration to several weeks to several months (e.g., 4 to 12 weeks). Multiple factors can influence the actual effective duration used for a particular application. For example, the severity of the cancer, the route of administration, the age and general health condition of the mammal, excipient usage, the possibility of co-usage with other therapeutic or prophylactic treatments such as use of other agents (e.g., checkpoint inhibitors), and the judgment of the treating physician may require an increase or decrease in the actual effective duration of administration of a composition provided herein (e.g., a pharmaceutical composition containing one or more binders provided herein).

In some cases, a binder (e.g., an antibody, antigen binding fragment, and/or antibody domain) provided herein can be used to detect the presence or absence of a PSMA polypeptide (e.g., a human PSMA polypeptide) in vitro, in situ, or in vivo (e.g., in vivo imaging within a mammal such as a human). For example, a binder (e.g., an antibody, antigen binding fragment, and/or antibody domain) provided herein can be designed to include a label (e.g., a covalently attached radioactive, enzymatic, colorimetric, or fluorescent label). The labelled binder can be used to detect the presence or absence of a PSMA polypeptide (e.g., a human PSMA polypeptide) within a biological sample in vitro. Examples of biological samples that can be assessed using a binder (e.g., an antibody, antigen binding fragment, and/or antibody domain) provided herein include, without limitation, serum samples, plasma samples, tissue samples, biopsy samples, cell line samples, and tissue culture samples. In some cases, a biological sample that can be assessed as described herein can include mammalian body tissues and/or cells such as leukocytes, ovary tissue or cells, prostate tissue or cells, heart tissue or cells, placenta tissue or cells, pancreas tissue or cells, liver tissue or cells, spleen tissue or cells, lung tissue or cells, breast tissue or cells, head and neck tissue or cells, endometrium tissue or cells, colon tissue or cells, colorectal tissue or cells, cervix tissue or cells, stomach tissue or cells, or umbilical tissue or cells that may express a PSMA polypeptide (e.g., a human PSMA polypeptide). In some cases, a binder (e.g., an antibody, antigen binding fragment, and/or antibody domain) provided herein can be immobilized, e.g., on a support, and retention of a PSMA polypeptide (e.g., a human PSMA polypeptide) from a biological sample on the support can be detected, and/or vice versa. In some cases, a binder (e.g., an antibody, antigen binding fragment, and/or antibody domain) provided herein can be used in applications such as fluorescence polarization, microscopy, ELISA, centrifugation, chromatography, and/or cell sorting (e.g., fluorescence activated cell sorting).

In some cases, a binder (e.g., an antibody, antigen binding fragment, and/or antibody domain) provided herein containing a label (e.g., a covalently attached radioactive label) can be used to detect the presence or absence of a PSMA polypeptide (e.g., a human PSMA polypeptide) within a mammal (e.g., a human). For example, a binder (e.g., an antibody, antigen binding fragment, and/or antibody domain) provided herein that is labelled (e.g., covalently labelled) with a radiolabel or an MRI detectable label can be administered to a mammal (e.g., a human), and that mammal can be assessed using a means for detecting the detectable label. In some cases, a mammal can be scanned to evaluate the location(s) of a labelled binder provided herein within the mammal. For example, the mammal can be imaged using NMR or other tomographic techniques.

Examples of labels that can be attached (e.g., covalently or non-covalently attached) to a binder (e.g., an antibody, antigen binding fragment, and/or antibody domain) provided herein include, without limitation, radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as a peroxidase or a phosphatase. In some cases, short-range radiation emitters such as isotopes detectable by short-range detector probes can be used.

The invention will be further described in the following examples, which do not limit the scope of the invention described in the claims.

### EXAMPLES

### Example 1—Obtaining Binders Having the Ability to Bind to a Human PSMA Polypeptide

Large phage displayed scFv and antibody domain libraries were panned and screened to identify binders that bind to a human PSMA polypeptide having the amino acid sequence set forth in SEQ ID NO:274 (FIG. 1). To identify such binders, a human PSMA polypeptide set forth in FIG. 1 was fused to the Fc region of human IgG1 at the C-terminus of the PSMA sequence, and the PSMA-Fc polypeptide was used for panning of human scFv and VH domain phage-displayed libraries. Two scFv's and nine VH domains (Clones: #1, #2, #3, #4, #5, #6, #7, #8, #9, #10, and #11, FIGS. 2-12) were identified.

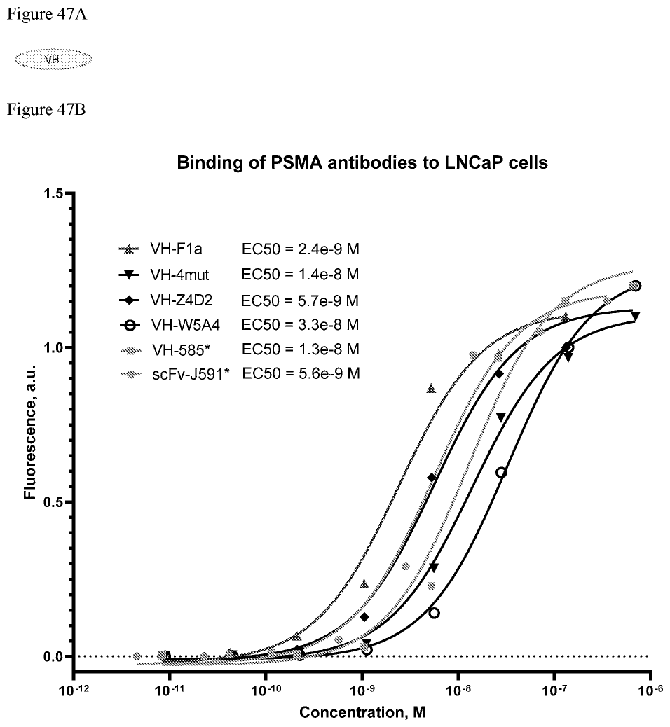

Binding affinity and specificity to a human PSMA polypeptide were tested using LNCaP cells expressing the PSMA protein. Clones #1-#11 exhibited affinity binding having EC₅₀ values of 6.56 nM, 8.97 nM, 4.59 nM, 3.05 nM, 2.95 nM, 32.7 nM, 8.65 nM, 10.8 nM, 15.1 nM, 8.70 nM, and 1.81 nM, respectively. All the clones bound to LNCaP cells displaying human PSMA, but did not bind to DU145 or PC3 cells lacking expression of human PSMA, demonstrating that they can bind to PSMA polypeptides present on cells. Clones #1-#11 competed with the J591 anti-PSMA antibody and the VH binder to PSMA from the bispecific antibody TNB-585 for binding to human PSMA expressed on LNCaP cells. In another experiment, additional binding results were obtained demonstrating that the indicated clones bound to LNCaP cells displaying human PSMA (FIGS. 47A and 47B).

### Example 2—Designing CARs from Binders Having the Ability to Bind to a Human PSMA Polypeptide

CARs were made using the sequences for Clones #1, #4, and #9. The constructed designs were as shown in FIGS. 29, 32, and 37, respectively.

### Example 3—Designing BiTEs from Binders Having the Ability to Bind to a Human PSMA Polypeptide

BiTEs were made using the sequences for Clones #1, #4, and #9. Clone #1 was a single chain variable fragment, and the BiTE using this clone had the format as shown in FIG. 40A. The sequences used in this design were as shown in FIG. 40C. A similar design can be used for Clone #2.

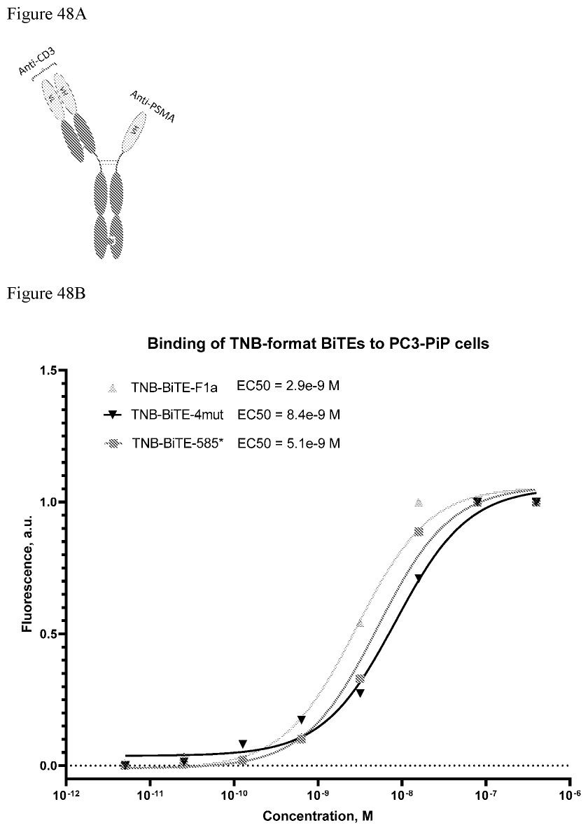

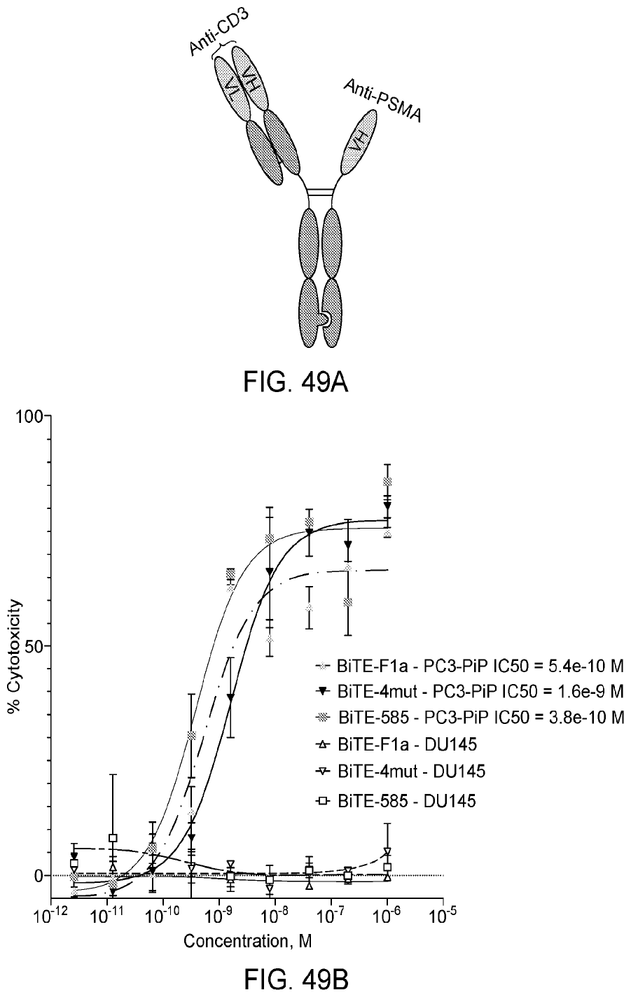

Clones #4 and #9 were VH single domains, and the format used for the BiTEs for these VH single domains were as shown in 40D. The sequences used for this design for Clone #4 and Clone #9 were as shown in FIGS. 40E and 40F, respectively. A similar design can be used for Clones #3, #5, #6, #7, #8, #10, and #11. The BiTEs designed for Clone #4 (FIG. 40E) and Clone #9 (FIG. 40F) bound to PSMA⁺ PC3-PiP cells (FIGS. 48A and 48B), and exhibited cell killing of PSMA⁺ PC3-PiP cells with little to no killing of PSMA⁻ DU145 cells (FIGS. 49A and 49B).

### Example 4—Designing BiKEs from Binders Having the Ability to Bind to a Human PSMA Polypeptide

The BiTE designs of Example 3 are used to construct BiKEs, where the CD3 binder sequence is replaced with a sequence of a binder to CD16a.

### Example 5—Designing ADCs from Binders Having the Ability to Bind to a Human PSMA Polypeptide

ADCs using the sequences of Clone #1 or Clone #2 are constructed using an IgG1 format. Drug payloads are conjugated to the antibodies using an enzyme-based labeling.

In one approach, conjugation to N297 is performed using a mutant galactosltransferase beta-1-4-galactotransferase bearing a mutation (e.g., Y289L) (see, e.g., International Patent Application Publication No. WO 2004/063344, U.S. Patent Application Publication No. 2021/0163623, or International Patent Application Publication No. WO 2017/132298.

In another approach, conjugation in performed using a bacterial transglutaminase to attach aminated linkers to the Q295 position of diglacosylated antibodies or antibodies bearing the N297A mutation (see, e.g., International Patent Application Publication No. WO 2019/057772).

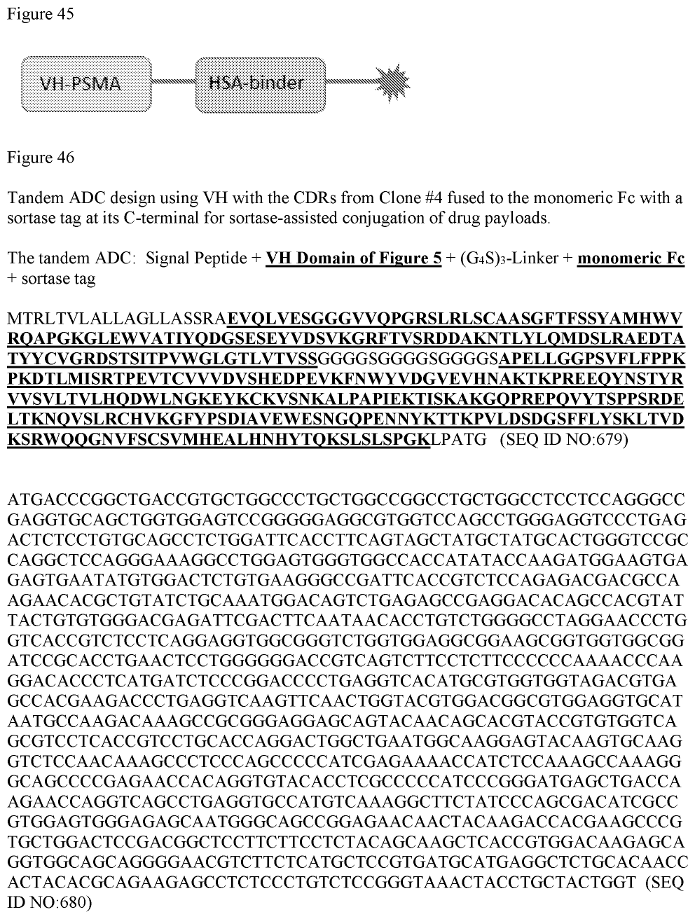

ADCs using VH domains (e.g., Clones #3-#11) are constructed using a tandem format as shown in FIG. 45, where an albumin binding fragment is a protein G albumin binding domain, an albumin binding nanobody, an albumin binding antibody domain (e.g., a VH or CH2-based albumin binder), or a monomeric Fc (mFc). An example of an ADC format using the sequence of Clone #4 and the monomeric Fc (mFc) is shown in FIG. 46. Drug payload is conjugated to the C-terminal of the fused polypeptides using a sortase tag.

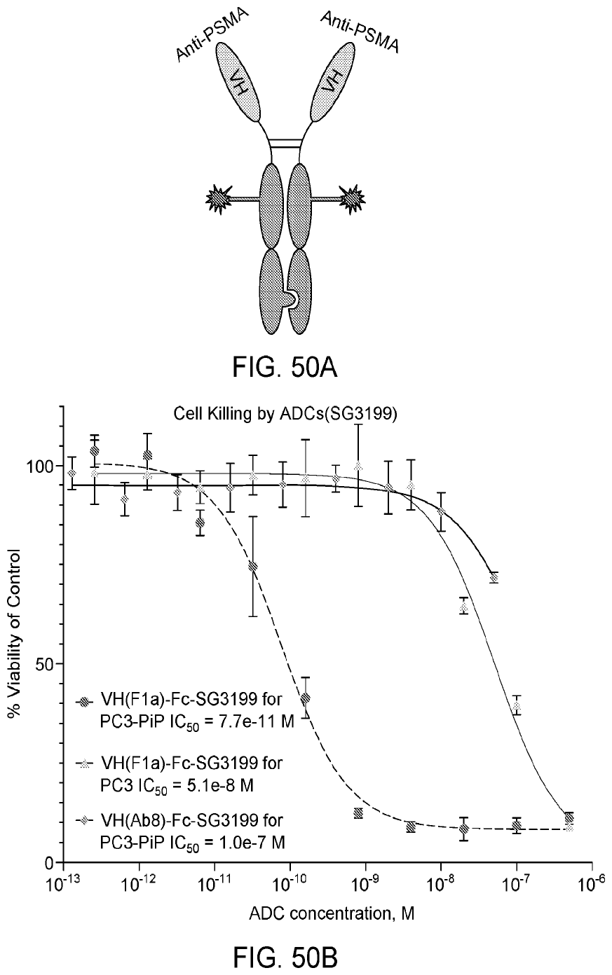

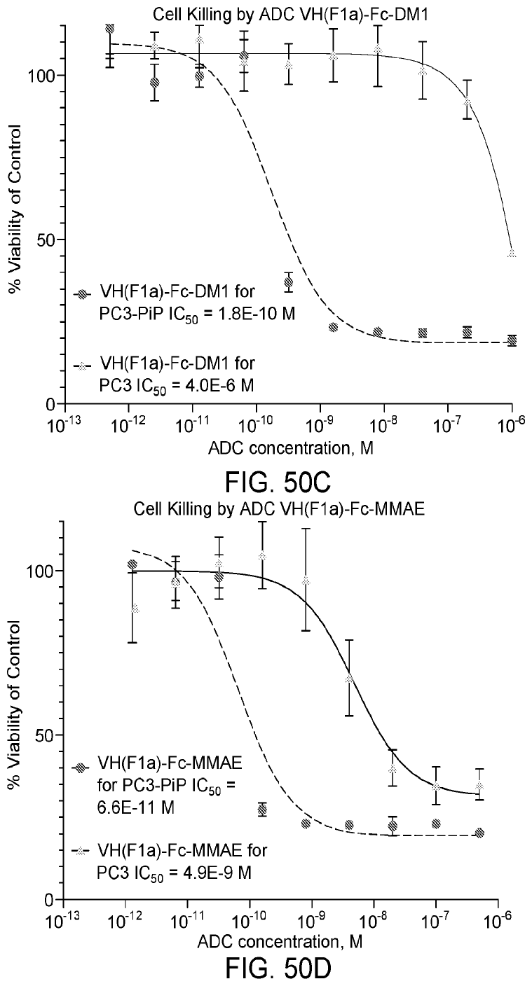

ADCs containing the sequence of Clone #4 and a drug (e.g., SG3199, DM-1, or MMAE) were created and tested for the ability to kill PSMA⁺ PC3-PiP cells or PSMA-PC3 cells (FIGS. 50A-50D). For each of the three tested drugs, the anti-PSMA ADCs killed PSMA⁺ PC3-PiP cells to a greater extent than the killing observed for PSMA⁻ PC3 cells (FIGS. 50B-50D).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions);
(iv) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(v) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(vi) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(viii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(ix) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions);
(x) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); or
(xi) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions).

2-28. (canceled)

29. An antigen binding fragment comprising:
(i) a heavy chain variable domain or region comprising the amino acid sequences set forth in SEQ ID NO:1 (or SEQ ID NO:1 with one, two, or three amino acid additions, deletions, or substitutions), SEQ ID NO:2 (or SEQ ID NO:2 with one, two, or three amino acid additions, deletions, or substitutions), and SEQ ID NO:3 (or SEQ ID NO:3 with one, two, or three amino acid additions, deletions, or substitutions), and a light chain variable domain or region comprising the amino acid sequences set forth in SEQ ID NO:9 (or SEQ ID NO:9 with one, two, or three amino acid additions, deletions, or substitutions), SEQ ID NO:10 (or SEQ ID NO:10 with one, two, or three amino acid additions, deletions, or substitutions), and SEQ ID NO:11 (or SEQ ID NO:11 with one, two, or three amino acid additions, deletions, or substitutions);
(ii) a heavy chain variable domain or region comprising the amino acid sequences set forth in SEQ ID NO:17 (or SEQ ID NO:17 with one, two, or three amino acid additions, deletions, or substitutions), SEQ ID NO:18 (or SEQ ID NO:18 with one, two, or three amino acid additions, deletions, or substitutions), and SEQ ID NO:19 (or SEQ ID NO:19 with one, two, or three amino acid additions, deletions, or substitutions), and a light chain variable domain or region comprising the amino acid sequences set forth in SEQ ID NO:25 (or SEQ ID NO:25 with one, two, or three amino acid additions, deletions, or substitutions), SEQ ID NO:26 (or SEQ ID NO:26 with one, two, or three amino acid additions, deletions, or substitutions), and SEQ ID NO:27 (or SEQ ID NO:27 with one, two, or three amino acid additions, deletions, or substitutions);
(iii) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions);
(iv) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(v) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(vi) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(vii) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(viii) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(ix) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions);
(x) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); or
(xi) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions).

30-60. (canceled)

61. An antibody domain comprising:
(i) a heavy chain variable domain or region comprising the amino acid sequences set forth in SEQ ID NO:33 (or SEQ ID NO:33 with one, two, or three amino acid additions, deletions, or substitutions), SEQ ID NO:34 (or SEQ ID NO:34 with one, two, or three amino acid additions, deletions, or substitutions), and SEQ ID NO:35 (or SEQ ID NO:35 with one, two, or three amino acid additions, deletions, or substitutions);
(ii) a heavy chain variable domain or region comprising the amino acid sequences set forth in SEQ ID NO:41 (or SEQ ID NO:41 with one, two, or three amino acid additions, deletions, or substitutions), SEQ ID NO:42 (or SEQ ID NO:42 with one, two, or three amino acid additions, deletions, or substitutions), and SEQ ID NO:43 (or SEQ ID NO:43 with one, two, or three amino acid additions, deletions, or substitutions);
(iii) a heavy chain variable domain or region comprising the amino acid sequences set forth in SEQ ID NO:49 (or SEQ ID NO:49 with one, two, or three amino acid additions, deletions, or substitutions), SEQ ID NO:50 (or SEQ ID NO:50 with one, two, or three amino acid additions, deletions, or substitutions), and SEQ ID NO:51 (or SEQ ID NO:51 with one, two, or three amino acid additions, deletions, or substitutions);
(iv) a heavy chain variable domain or region comprising the amino acid sequences set forth in SEQ ID NO:57 (or SEQ ID NO:57 with one, two, or three amino acid additions, deletions, or substitutions), SEQ ID NO:58 (or SEQ ID NO:58 with one, two, or three amino acid additions, deletions, or substitutions), and SEQ ID NO:59 (or SEQ ID NO:59 with one, two, or three amino acid additions, deletions, or substitutions);
(v) a heavy chain variable domain or region comprising the amino acid sequences set forth in SEQ ID NO:65 (or SEQ ID NO:65 with one, two, or three amino acid additions, deletions, or substitutions), SEQ ID NO:66 (or SEQ ID NO:66 with one, two, or three amino acid additions, deletions, or substitutions), and SEQ ID NO:67 (or SEQ ID NO:67 with one, two, or three amino acid additions, deletions, or substitutions);
(vi) a heavy chain variable domain or region comprising the amino acid sequences set forth in SEQ ID NO:73 (or SEQ ID NO:73 with one, two, or three amino acid additions, deletions, or substitutions), SEQ ID NO:74 (or SEQ ID NO:74 with one, two, or three amino acid additions, deletions, or substitutions), and SEQ ID NO:75 (or SEQ ID NO:75 with one, two, or three amino acid additions, deletions, or substitutions);
(vii) a heavy chain variable domain or region comprising the amino acid sequences set forth in SEQ ID NO:81 (or SEQ ID NO:81 with one, two, or three amino acid additions, deletions, or substitutions), SEQ ID NO:82 (or SEQ ID NO:82 with one, two, or three amino acid additions, deletions, or substitutions), and SEQ ID NO:83 (or SEQ ID NO:83 with one, two, or three amino acid additions, deletions, or substitutions);
(viii) a heavy chain variable domain or region comprising the amino acid sequences set forth in SEQ ID NO:89 (or SEQ ID NO:89 with one, two, or three amino acid additions, deletions, or substitutions), SEQ ID NO:90 (or SEQ ID NO:90 with one, two, or three amino acid additions, deletions, or substitutions), and SEQ ID NO:91 (or SEQ ID NO:91 with one, two, or three amino acid additions, deletions, or substitutions); or
(ix) a heavy chain variable domain or region comprising the amino acid sequences set forth in SEQ ID NO:97 (or SEQ ID NO:97 with one, two, or three amino acid additions, deletions, or substitutions), SEQ ID NO:98 (or SEQ ID NO:98 with one, two, or three amino acid additions, deletions, or substitutions), and SEQ ID NO:99 (or SEQ ID NO:99 with one, two, or three amino acid additions, deletions, or substitutions).

62. The antibody domain of claim 61, wherein said antibody domain comprises the ability to bind to SEQ ID NO:273 or SEQ ID NO:274.

63. The antibody domain of claim 61, wherein said antibody domain comprises said heavy chain variable domain or region of said (i).

64. (canceled)

65. The antibody domain of claim 61, wherein said antibody domain comprises said heavy chain variable domain or region of said (ii).

66. (canceled)

67. The antibody domain of claim 61, wherein said antibody domain comprises said heavy chain variable domain or region of said (iii).

68. (canceled)

69. The antibody domain of claim 61, wherein said antibody domain comprises said heavy chain variable domain or region of said (iv).

70. (canceled)

71. The antibody domain of claim 61, wherein said antibody domain comprises said heavy chain variable domain or region of said (v).

72. (canceled)

73. The antibody domain of claim 61, wherein said antibody domain comprises said heavy chain variable domain or region of said (vi).

74. (canceled)

75. The antibody domain of claim 61, wherein said antibody domain comprises said heavy chain variable domain or region of said (vii).

76. (canceled)

77. The antibody domain of claim 61, wherein said antibody domain comprises said heavy chain variable domain or region of said (viii).

78. (canceled)

79. The antibody domain of claim 61, wherein said antibody domain comprises said heavy chain variable domain or region of said (ix).

80-82. (canceled)

83. A chimeric antigen receptor comprising an antigen binding domain, a hinge, a transmembrane domain, and one or more signaling domains, wherein said antigen binding domain comprises an antibody, an antigen-binding fragment, or an antibody domain of any one of claims 1, 29, and 61.

84-90. (canceled)

91. A cell engager comprising a first antigen binding domain, a linker, and a second antigen binding domain, wherein said first antigen binding domain comprises an antibody, an antigen-binding fragment, or an antibody domain of any one of claims 1, 29, and 61.

92-105. (canceled)

106. A nucleic acid comprising a nucleic acid sequence encoding at least part of said antibody, said antigen-binding fragment, or said antibody domain of any one of claims 1, 29, and 61.

107-117. (canceled)

118. An antibody-drug conjugate (ADC) comprising an antigen binging domain covalently linked to a drug, wherein said antigen binging domain comprises an antibody, an antigen binding fragment, or an antibody domain of any one of claims 1, 29, and 61.

119-122. (canceled)

123. A composition comprising an antibody of claim 1, an antigen binding fragment of claim 29, an antibody domain of claim 61, a cell engager of claim 91, or an ADC of claim 118.

124-131. (canceled)

132. A method of treating a mammal having cancer, wherein said method comprises administering, to said mammal, a composition of claim 123.

133-142. (canceled)

143. A method for binding a binding molecule to a PSMA polypeptide, wherein said method comprises contacting said PSMA polypeptide with an antibody of claim 1, an antigen binding fragment of claim 29, an antibody domain of claim 61, a chimeric antigen receptor of claim 83, a cell engager of claim 91, or an ADC of claim 118.

144-152. (canceled)


SEQUENCE LISTING (680 sequences)

SEQ ID NO: 1 - protein, 8 aa, synthetic construct
GYSFTNYW

SEQ ID NO: 2 - protein, 8 aa, synthetic construct
IYPGDSDT

SEQ ID NO: 3 - protein, 12 aa, synthetic construct
ARQGGYYYGMDV

SEQ ID NO: 4 - protein, 25 aa, synthetic construct
QVTLKESGAEVKKPGESLKISCKAS

SEQ ID NO: 5 - protein, 17 aa, synthetic construct
IGWVRQMPGKGLEWMGI

SEQ ID NO: 6 - protein, 38 aa, synthetic construct
RYSPSFQGQVTISADKSISTAYLQWSSLKASDTAMYYC

SEQ ID NO: 7 - protein, 11 aa, synthetic construct
WGQGTTVTVSS

SEQ ID NO: 8 - protein, 119 aa, synthetic construct
QVTLKESGAEVKKPGESLKISCKASGYSFTNYWIGWVRQMPGKGLEWMGIIYPGDSDTRYSPSFQGQVTISADKSISTAYLQWSSLKASDTAMYYCARQGGYYYGMDVWGQGTTVTVSS

SEQ ID NO: 9 - protein, 8 aa, synthetic construct
SGSIASNY

SEQ ID NO: 10 - skipped

SEQ ID NO: 11 - protein, 10 aa, synthetic construct
QSYDSRNHVV

SEQ ID NO: 12 - protein, 25 aa, synthetic construct
VPVLTQPHSVSESPGKTVTISCTRS

SEQ ID NO: 13 - protein, 17 aa, synthetic construct
VQWYQQRPGSSPTTVIY

SEQ ID NO: 14 - protein, 38 aa, synthetic construct
QRPSGVPDRFSGSIDMSSNSASLTISGLRTEDEGDYYC

SEQ ID NO: 15 - protein, 11 aa, synthetic construct
FGGGTQLTVLG

SEQ ID NO: 16 - protein, 112 aa, synthetic construct
VPVLTQPHSVSESPGKTVTISCTRSSGSIASNYVQWYQQRPGSSPTTVIYEDNQRPSGVPDRFSGSIDMSSNSASLTISGLRTEDEGDYYCQSYDSRNHVVFGGGTQLTVLG

SEQ ID NO: 17 - protein, 8 aa, synthetic construct
GYSFTNHW

SEQ ID NO: 18 - protein, 8 aa, synthetic construct
IYPGDSDT

SEQ ID NO: 19 - protein, 10 aa, synthetic construct
ARAQVGAFDI

SEQ ID NO: 20 - protein, 25 aa, synthetic construct
QVQLQQSGAEVKKPGESLKISCKGS

SEQ ID NO: 21 - protein, 17 aa, synthetic construct
IAWVRQMPGKGLEWMGI

SEQ ID NO: 22 - protein, 38 aa, synthetic construct; identical to SEQ ID NO: 6

SEQ ID NO: 23 - protein, 11 aa, synthetic construct
WGQGTMVTVSS

SEQ ID NO: 24 - protein, 117 aa, synthetic construct
QVQLQQSGAEVKKPGESLKISCKGSGYSFTNHWIAWVRQMPGKGLEWMGIIYPGDSDTRYSPSFQGQVTISADKSISTAYLQWSSLKASDTAMYYCARAQVGAFDIWGQGTMVTVSS

SEQ ID NO: 25 - protein, 9 aa, synthetic construct
RDDVGGDNF

SEQ ID NO: 26 - skipped

SEQ ID NO: 27 - protein, 11 aa, synthetic construct
SSYTSSSTLPV

SEQ ID NO: 28 - protein, 25 aa, synthetic construct
QSALTQPASVSGSPGQSITISCTRA

SEQ ID NO: 29 - protein, 17 aa, synthetic construct
VSWYQQHPGKAPKLMIY

SEQ ID NO: 30 - protein, 36 aa, synthetic construct
NRPSGVSNRFSGSKSGNTASLTISGLQAEDEADYYC

SEQ ID NO: 31 - protein, 11 aa, synthetic construct
FGGGTQLTVLG

SEQ ID NO: 32 - protein, 112 aa, synthetic construct
QSALTQPASVSGSPGQSITISCTRARDDVGGDNFVSWYQQHPGKAPKLMIYDVSNRPSGVSNRFSGSKSGNTASLTISGLQAEDEADYYCSSYTSSSTLPVFGGGTQLTVLG

SEQ ID NO: 33 - protein, 8 aa, synthetic construct
GFTFSSYA

SEQ ID NO: 34 - protein, 8 aa, synthetic construct
IYQDGSES

SEQ ID NO: 35 - protein, 11 aa, synthetic construct
VGRGSTSITPV

SEQ ID NO: 36 - protein, 25 aa, synthetic construct
EVQLVESGGGVVQPGRSLRLSCAAS

SEQ ID NO: 37 - protein, 17 aa, synthetic construct
MHWVRQAPGKGLEWVAT

SEQ ID NO: 38 - protein, 38 aa, synthetic construct
EYVDSVKGRFTVSRDNAKNTLYLQMDSLRAEDTATYYC

SEQ ID NO: 39 - protein, 11 aa, synthetic construct
WGLGTLVTVSS

SEQ ID NO: 40 - protein, 118 aa, synthetic construct
EVQLVESGGGVVQPGRSLRLSCAASGFTFSSYAMHWVRQAPGKGLEWVATIYQDGSESEYVDSVKGRFTVSRDNAKNTLYLQMDSLRAEDTATYYCVGRGSTSITPVWGLGTLVTVSS

SEQ ID NO: 41 - protein, 8 aa, synthetic construct
GFTFSSYA

SEQ ID NO: 42 - protein, 8 aa, synthetic construct
IYQDGSES

SEQ ID NO: 43 - protein, 11 aa, synthetic construct
VGRDSTSITPV

SEQ ID NO: 44 - protein, 25 aa, synthetic construct
EVQLVESGGGVVQPGRSLRLSCAAS

SEQ ID NO: 45 - protein, 17 aa, synthetic construct
MHWVRQAPGKGLEWVAT

SEQ ID NO: 46 - protein, 38 aa, synthetic construct
EYVDSVKGRFTVSRDDAKNTLYLQMDSLRAEDTATYYC

SEQ ID NO: 47 - protein, 11 aa, synthetic construct
WGLGTLVTVSS

SEQ ID NO: 48 - protein, 118 aa, synthetic construct
EVQLVESGGGVVQPGRSLRLSCAASGFTFSSYAMHWVRQAPGKGLEWVATIYQDGSESEYVDSVKGRFTVSRDDAKNTLYLQMDSLRAEDTATYYCVGRDSTSITPVWGLGTLVTVSS

SEQ ID NO: 49 - protein, 8 aa, synthetic construct
GFTFSSYA

SEQ ID NO: 50 - protein, 8 aa, synthetic construct
IYQDGSES

SEQ ID NO: 51 - protein, 11 aa, synthetic construct
VGRASASITPV

SEQ ID NO: 52 - protein, 25 aa, synthetic construct
EVQLVESGGGMVQPGRSLRLSCAAS

SEQ ID NO: 53 - protein, 17 aa, synthetic construct
MHWIRQAPGKGLEWVAT

SEQ ID NO: 54 - protein, 38 aa, synthetic construct
EYVDSVKGRFAVSRDNAKNTLYLQMDSLRAEDTATYYC

SEQ ID NO: 55 - protein, 11 aa, synthetic construct
WGLGTLVTVSS

SEQ ID NO: 56 - protein, 118 aa, synthetic construct
EVQLVESGGGMVQPGRSLRLSCAASGFTFSSYAMHWIRQAPGKGLEWVATIYQDGSESEYVDSVKGRFAVSRDNAKNTLYLQMDSLRAEDTATYYCVGRASASITPVWGLGTLVTVSS

SEQ ID NO: 57 - protein, 8 aa, synthetic construct
GFTFSSYA

SEQ ID NO: 58 - protein, 7 aa, synthetic construct
IGAGGGT

SEQ ID NO: 59 - protein, 6 aa, synthetic construct
AKDGAD

SEQ ID NO: 60 - protein, 25 aa, synthetic construct
EVQLVESGGGLVQPGGSLRLSCTVS

SEQ ID NO: 61 - protein, 17 aa, synthetic construct
VSWVRQAPGQGLEWVSA

SEQ ID NO: 62 - protein, 38 aa, synthetic construct
YYADSVKGRFTISRDNSKNTLYLQMNSLRAEDTAVYYC

SEQ ID NO: 63 - protein, 12 aa, synthetic construct
WGQGTLVTVSSS

SEQ ID NO: 64 - protein, 113 aa, synthetic construct
EVQLVESGGGLVQPGGSLRLSCTVSGFTFSSYAVSWVRQAPGQGLEWVSAIGAGGGTYYADSVKGRFTISRDNSKNTLYLQMNSLRAEDTAVYYCAKDGADWGQGTLVTVSSS

SEQ ID NO: 65 - protein, 8 aa, synthetic construct
GFTFSSYA

SEQ ID NO: 66 - protein, 7 aa, synthetic construct
IGAGGGT

SEQ ID NO: 67 - protein, 6 aa, synthetic construct
AKDGVD

SEQ ID NO: 68 - protein, 25 aa, synthetic construct
EVQLVESGGGLVQPGGSLRLSCTVS

SEQ ID NO: 69 - protein, 17 aa, synthetic construct
VSWVRQAPGQGLEWVSA

SEQ ID NO: 70 - protein, 38 aa, synthetic construct; identical to SEQ ID NO: 62

SEQ ID NO: 71 - protein, 12 aa, synthetic construct
WGQGTLVTVSSS

SEQ ID NO: 72 - protein, 113 aa, synthetic construct
EVQLVESGGGLVQPGGSLRLSCTVSGFTFSSYAVSWVRQAPGQGLEWVSAIGAGGGTYYADSVKGRFTISRDNSKNTLYLQMNSLRAEDTAVYYCAKDGVDWGQGTLVTVSSS

SEQ ID NO: 73 - protein, 8 aa, synthetic construct
GFTFSSYA

SEQ ID NO: 74 - protein, 7 aa, synthetic construct
IGAGGGT

SEQ ID NO: 75 - protein, 6 aa, synthetic construct
AKDGAD

SEQ ID NO: 76 - protein, 25 aa, synthetic construct
EVQLVESGGGLVQPGGSLRLSCTVS

SEQ ID NO: 77 - protein, 17 aa, synthetic construct
VSWVRQAPGQGLEWVSS

SEQ ID NO: 78 - protein, 38 aa, synthetic construct; identical to SEQ ID NO: 62

SEQ ID NO: 79 - protein, 12 aa, synthetic construct
WGQGTLVTVSSS

SEQ ID NO: 80 - protein, 113 aa, synthetic construct
EVQLVESGGGLVQPGGSLRLSCTVSGFTFSSYAVSWVRQAPGQGLEWVSSIGAGGGTYYADSVKGRFTISRDNSKNTLYLQMNSLRAEDTAVYYCAKDGADWGQGTLVTVSSS

SEQ ID NO: 81 - protein, 8 aa, synthetic construct
GFTFDDYA

SEQ ID NO: 82 - protein, 7 aa, synthetic construct
IGAGGGT

SEQ ID NO: 83 - protein, 6 aa, synthetic construct
AKDGVD

SEQ ID NO: 84 - protein, 25 aa, synthetic construct
EVQLVESGGGLVQPGGSLRLSCTVS

SEQ ID NO: 85 - protein, 17 aa, synthetic construct
VSWVRQAPGQGLEWVSS

SEQ ID NO: 86 - protein, 38 aa, synthetic construct
YYADSVKGRFTISRDDSKDTLYLQMNSLRAEDTAVYYC

SEQ ID NO: 87 - protein, 12 aa, synthetic construct
WGQGTLVTVSSS

SEQ ID NO: 88 - protein, 113 aa, synthetic construct
EVQLVESGGGLVQPGGSLRLSCTVSGFTFDDYAVSWVRQAPGQGLEWVSSIGAGGGTYYADSVKGRFTISRDDSKDTLYLQMNSLRAEDTAVYYCAKDGVDWGQGTLVTVSSS

SEQ ID NO: 89 - protein, 8 aa, synthetic construct
GFTFSSYA

SEQ ID NO: 90 - protein, 8 aa, synthetic construct
IYQDGSES

SEQ ID NO: 91 - protein, 11 aa, synthetic construct
VGRDSTSITPV

SEQ ID NO: 92 - protein, 25 aa, synthetic construct
EVQMVESGGGVVQPGRSLRLSCAAS

SEQ ID NO: 93 - protein, 17 aa, synthetic construct
MHWVRQAPGKGLEWVAT

SEQ ID NO: 94 - protein, 38 aa, synthetic construct
EYVDSVKGRFTVSIDNAKNTLYLQMDSLRAEDTATYYC

SEQ ID NO: 95 - protein, 11 aa, synthetic construct
WGLGTLVTVSS

SEQ ID NO: 96 - protein, 118 aa, synthetic construct
EVQMVESGGGVVQPGRSLRLSCAASGFTFSSYAMHWVRQAPGKGLEWVATIYQDGSESEYVDSVKGRFTVSIDNAKNTLYLQMDSLRAEDTATYYCVGRDSTSITPVWGLGTLVTVSS

SEQ ID NO: 97 - protein, 8 aa, synthetic construct
GFTFSSYA

SEQ ID NO: 98 - protein, 8 aa, synthetic construct
IYQDGSES

SEQ ID NO: 99 - protein, 11 aa, synthetic construct
VGRASTSITPV

SEQ ID NO: 100 - protein, 25 aa, synthetic construct
EVQLVESGGGVVQPGRSLRLSCAAS

SEQ ID NO: 101 - protein, 17 aa, synthetic construct
MHWVRQAPGKGLEWVAT

SEQ ID NO: 102 - protein, 38 aa, synthetic construct; identical to SEQ ID NO: 94

SEQ ID NO: 103 - protein, 11 aa, synthetic construct
WGLGTLVTVSS

SEQ ID NO: 104 - protein, 118 aa, synthetic construct
EVQLVESGGGVVQPGRSLRLSCAASGFTFSSYAMHWVRQAPGKGLEWVATIYQDGSESEYVDSVKGRFTVSIDNAKNTLYLQMDSLRAEDTATYYCVGRASTSITPVWGLGTLVTVSS

SEQ ID NO: 105 - DNA, 357 bp, synthetic construct
  source 1..357: mol_type = other DNA
caggtcaccttgaaggagtctggggcagaggtgaaaaagccgggggagtctctgaaaatctcctgtaaggcttctggatacagctttaccaattactggatcggctgggtgcgccagatgcccggcaaaggcctggagtggatggggatcatctatcctggtgactctgataccagatacagcccgtccttccaaggccaggtcaccatctcagccgacaagtccatcagcactgcctacctgcagtggagcagcctgaaggcctcggacaccgccatgtattactgtgcgagacaggggggctactactacggtatggacgtctggggccaagggaccacggtcaccgtctcctca

SEQ ID NO: 106 - DNA, 45 bp, synthetic construct
  source 1..45: mol_type = other DNA
ggaggtggcgggtctggtggtagcggaagcggtggtggcggatcc

SEQ ID NO: 107 - DNA, 336 bp, synthetic construct
  source 1..336: mol_type = other DNA
gtgcctgtgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacatgtcgtccaactctgcctccctcaccatctctggactgaggactgaggacgagggtgactattactgtcagtcttatgatagccgcaatcatgtggtattcggcggagggacccagctcaccgttttaggt

SEQ ID NO: 108 - DNA, 351 bp, synthetic construct
  source 1..351: mol_type = other DNA
caggtacagctgcagcagtcaggagcagaggtgaaaaagcccggggagtctctgaagatctcctgtaagggttctggatacagctttaccaaccactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagagctcaagtcggggcttttgatatctggggccaagggacaatggtcaccgtctcttca

SEQ ID NO: 109 - DNA, 45 bp, synthetic construct
  source 1..45: mol_type = other DNA
ggaggtggcgggtctggtggtagcggaagcagtggtggcggatcc

SEQ ID NO: 110 - DNA, 336 bp, synthetic construct
  source 1..336: mol_type = other DNA
cagtctgccctgactcagcctgcctccgtgtctgggtctcctggacagtcgatcaccatctcctgcactagagccagggatgacgttggtggtgacaactttgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactcttccggtgttcggcgggggcacccagctgaccgtcctcggt

SEQ ID NO: 111 - DNA, 354 bp, synthetic construct
  source 1..354: mol_type = other DNA
gaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccagagacaacgccaagaacacgctgtatctgcaaatggacagtctgagagccgaggacacagccacgtattactgtgtgggacgcggttcgacttcaataacacctgtctggggcctaggaaccctggtcaccgtctcctca

SEQ ID NO: 112 - DNA, 357 bp, synthetic construct
  source 1..357: mol_type = other DNA
gaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccagagacgacgccaagaacacgctgtatctgcaaatggacagtctgagagccgaggacacagccacgtattactgtgtgggacgagattcgacttcaataacacctgtctggggcctaggaaccctggtcaccgtctcctcaagt

SEQ ID NO: 113 - DNA, 357 bp, synthetic construct
  source 1..357: mol_type = other DNA
gaggtgcagctggtggagtccgggggaggcatggtccagcctgggaggtccctgagactctcctgtgcagcctctggattcaccttcagtagctatgctatgcactggatccgccaggctccagggaaaggcctggagtgggtggccaccatataccaagatggaagtgagagtgaatatgtagactctgtgaagggccgattcgccgtctccagagacaacgccaagaacacgctgtatctgcaaatggacagtctgagagccgaggacacagccacgtattactgtgtgggacgcgcttcggcttcaataacacctgtctggggcctaggaaccctggtcaccgtctcctcaagt

SEQ ID NO: 114 - DNA, 339 bp, synthetic construct
  source 1..339: mol_type = other DNA
gaggtgcagctggtggagtctgggggaggcttggtacagcctggagggtccctgagactctcctgtaccgtctctggattcacctttagcagctatgccgtgagctgggtccgccaggctccaggacaagggcttgagtgggtatcagctattggtgctggtggtggcacatactacgcagactctgtgaagggccgattcaccatttccagagacaattccaagaacacgctgtatctgcaaatgaacagcctgagagccgaggacacagccgtgtattactgtgcgaaagacggggccgactggggccagggaaccctggtcaccgtctcctcaagt

SEQ ID NO: 115 - DNA, 339 bp, synthetic construct
  source 1..339: mol_type = other DNA
gaggtgcagctggtggagtctgggggaggcttggtacagcctggagggtccctgagactctcctgtaccgtctctggattcacctttagcagctatgccgtgagctgggtccgccaggctccaggacaagggcttgagtgggtatcagctattggtgctggtggtggcacatactacgcagactctgtgaagggccgattcaccatttccagagacaattccaagaacacgctgtatctgcaaatgaacagcctgagagccgaggacacagccgtgtattactgtgcgaaagacggggtcgactggggccagggaaccctggtcaccgtctcctcaagt

SEQ ID NO: 116 - DNA, 339 bp, synthetic construct
  source 1..339: mol_type = other DNA
gaggtgcagctggtggagtctgggggaggcttggtacagcccggagggtccctgagactctcctgtaccgtctctggattcacctttagcagctatgccgtgagctgggtccgccaggctccaggacaagggcttgagtgggtatcatctattggtgctggtggtggcacatactacgcagactctgtgaagggccgattcaccatttccagagacaattccaagaacacgctgtatctgcaaatgaacagcctgagagccgaggacacagccgtgtattactgtgcgaaagacggggccgactggggccagggaaccctggtcaccgtctcctcaagt

SEQ ID NO: 117 - DNA, 339 bp, synthetic construct
  source 1..339: mol_type = other DNA
gaggtgcagctggtggagtctgggggaggcttggtacagcccggagggtccctgagactctcctgtaccgtctctggattcacctttgacgactatgccgtgagctgggtccgccaggctccaggacaagggcttgagtgggtatcatctattggtgctggtggtggcacatactacgcagactctgtgaagggccgattcaccatttccagagacgattccaaggacacgctgtatctgcaaatgaacagcctgagagccgaggacacagccgtgtattactgtgcgaaagacggggtcgactggggccagggaaccctggtcaccgtctcctcaagt

SEQ ID NO: 118 - DNA, 357 bp, synthetic construct
  source 1..357: mol_type = other DNA
gaggtgcagatggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccatagacaacgccaagaacacgctgtatctgcaaatggacagtctgagagccgaggacacagccacgtattactgtgtgggacgcgattcgacttcaataacacctgtctggggcctaggaaccctggtcaccgtctcctcaagt

SEQ ID NO: 119 - DNA, 357 bp, synthetic construct
  source 1..357: mol_type = other DNA
gaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccatagacaacgccaagaacacgctgtatctgcaaatggacagtctgagagccgaggacacagccacgtattactgtgtgggacgcgcttcgacttcaataacacctgtctggggcctaggaaccctggtcaccgtctcctcaagt

SEQ ID NO: 120 - protein, 15 aa, synthetic construct
EPKSCDKTHTCPPCP

SEQ ID NO: 121 - DNA, 45 bp, synthetic construct
  source 1..45: mol_type = other DNA
gagcccaaatcttgtgacaaaactcacacatgcccaccgtgccca

SEQ ID NO: 122 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - DNA, 651 bp, synthetic construct
  source 1..651: mol_type = other DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 124-128 - skipped (5 entries)

SEQ ID NO: 129 - protein, 30 aa, synthetic construct
QVQLVESGGGLVKPGGSLRLSCAASGFTFS

SEQ ID NO: 130 - protein, 14 aa, synthetic construct
WIRQAPGKGLEWVS

SEQ ID NO: 131 - protein, 32 aa, synthetic construct
RFTISRDNAKNSLYLQMNSLRAEDTAVYYCAR

SEQ ID NO: 132 - protein, 11 aa, synthetic construct
WGQGTTVTVSS

SEQ ID NO: 133 - skipped

SEQ ID NO: 134 - protein, 23 aa, synthetic construct
DIQMTQSPSSLSASVGDRVTITC

SEQ ID NO: 135 - protein, 15 aa, synthetic construct
WYQQKPGKAPKLLIY

SEQ ID NO: 136 - protein, 32 aa, synthetic construct
GVPSRFSGSGSGTDFTLTISSLQPEDFGVYYC

SEQ ID NO: 137 - protein, 11 aa, synthetic construct
FGQGTRLEIKR

SEQ ID NO: 138 - skipped

SEQ ID NO: 139 - protein, 30 aa, synthetic construct; identical to SEQ ID NO: 129

SEQ ID NO: 140 - protein, 14 aa, synthetic construct
WIRQAPGKGLEWVS

SEQ ID NO: 141 - protein, 32 aa, synthetic construct
RFTISRDNAKNSLYLQMNSLRAEDTAMYYCAR

SEQ ID NO: 142 - protein, 11 aa, synthetic construct
WGQGTTVTVSS

SEQ ID NO: 143 - skipped

SEQ ID NO: 144 - protein, 23 aa, synthetic construct
DIQMTQSPSSLSASVGDRVTITC

SEQ ID NO: 145 - protein, 15 aa, synthetic construct
WYQQKPGKAPKLLIY

SEQ ID NO: 146 - protein, 32 aa, synthetic construct
GVPSRFSGSGSGTDFTLTISSLQPEDVGVYYC

SEQ ID NO: 147 - protein, 11 aa, synthetic construct
FGGGTKLEIKR

SEQ ID NO: 148 - skipped

SEQ ID NO: 149 - protein, 25 aa, synthetic construct
EVQLVQSGGGLVQPGGSLRLSCSAS

SEQ ID NO: 150 - protein, 17 aa, synthetic construct
MHWVRQAPGKGLEYVSA

SEQ ID NO: 151 - protein, 38 aa, synthetic construct; identical to SEQ ID NO: 62

SEQ ID NO: 152 - protein, 11 aa, synthetic construct
WGQGTMVTVSS

SEQ ID NO: 153 - skipped

SEQ ID NO: 154 - protein, 26 aa, synthetic construct
EATLTQSPSSLSASVGDRVTITCRAS

SEQ ID NO: 155 - protein, 17 aa, synthetic construct
LNWYQQKPGRAPKLLIY

SEQ ID NO: 156 - protein, 36 aa, synthetic construct
SLQSGVPSRFSGSGSGTDFTLTISSLQPEDFATYYC

SEQ ID NO: 157 - protein, 11 aa, synthetic construct
FGQGTRLEIKR

SEQ ID NO: 158 - skipped

SEQ ID NO: 159 - protein, 25 aa, synthetic construct
EVQLVQSGGGLVQPGGSLRLSCAAS

SEQ ID NO: 160 - protein, 17 aa, synthetic construct
MTWVRQAPGKGLEWVST

SEQ ID NO: 161 - protein, 38 aa, synthetic construct
YYADSVKGRFTISRDNDKNTLYLQMNSLRADDTAVYYC

SEQ ID NO: 162 - protein, 11 aa, synthetic construct
WGQGTLVTVSS

SEQ ID NO: 163 - skipped

SEQ ID NO: 164 - protein, 25 aa, synthetic construct
QSVVTQPPSASGSPGQSVTISCTGT

SEQ ID NO: 165 - protein, 17 aa, synthetic construct
VSWYQQHPGKAPKLLIY

SEQ ID NO: 166 - protein, 36 aa, synthetic construct
ERPSGVPDRFSGSKSDNKASLTIIGLQTEDEADYYC

SEQ ID NO: 167 - protein, 11 aa, synthetic construct
FGTGTKLTVLR

SEQ ID NO: 168 - protein, 15 aa, synthetic construct
GGGGSGGSGSGGGGS

SEQ ID NO: 169 - protein, 15 aa, synthetic construct
GGGGSGGSGSSGGGS

SEQ ID NO: 170 - protein, 15 aa, synthetic construct
GGGGSGGGGSGGGGS

SEQ ID NO: 171 - protein, 15 aa, synthetic construct
GGGGSGSGASSGGGS

SEQ ID NO: 172 - protein, 15 aa, synthetic construct
GGGGSGSGASGGGGS

SEQ ID NO: 173 - protein, 15 aa, synthetic construct
SGGGSGGGGSGGGGS

SEQ ID NO: 174 - protein, 18 aa, synthetic construct
GSTSGSGKPGSGEGSTKG

SEQ ID NO: 175 - protein, 19 aa, synthetic construct
PNGASQSSSASHTGSAPGS

SEQ ID NO: 176 - protein, 19 aa, synthetic construct
PNGASNSGSAPDTSSAPGS

SEQ ID NO: 177 - protein, 19 aa, synthetic construct
PNGASHSGSAPNTSSAPGS

SEQ ID NO: 178 - protein, 19 aa, synthetic construct
PNGASESGSASKTSSASGS

SEQ ID NO: 179 - protein, 19 aa, synthetic construct
PNGASNSGSAPKTGSASGS

SEQ ID NO: 180 - protein, 19 aa, synthetic construct
PNGASKSGSASQTSSAPGS

SEQ ID NO: 181 - protein, 19 aa, synthetic construct
PNGASHSSSASQTGSAPGS

SEQ ID NO: 182 - protein, 12 aa, synthetic construct
PNGASKRSAPGS

SEQ ID NO: 183 - protein, 19 aa, synthetic construct
PNGASHSGSAPHTSSASGS

SEQ ID NO: 184 - protein, 15 aa, synthetic construct
RGRGRGRGRSRGGGS

SEQ ID NO: 185 - protein, 15 aa, synthetic construct
SHGGSHGGGSGGGGS

SEQ ID NO: 186 - protein, 5 aa, synthetic construct
GQAGR

SEQ ID NO: 187 - protein, 8 aa, synthetic construct
GGGSGGGG

SEQ ID NO: 188 - protein, 5 aa, synthetic construct
SGGGG

SEQ ID NO: 189 - protein, 5 aa, synthetic construct
GGGGS

SEQ ID NO: 190 - protein, 30 aa, synthetic construct
GGGGSGGGGSGGGGSGGGGSGGGGSGGGGS

SEQ ID NO: 191 - protein, 16 aa, synthetic construct
GGGGGSGGGGSGGGGS

SEQ ID NO: 192 - protein, 26 aa, synthetic construct
GGGGGSGGGGSGGGGSGGGGSGGGGS

SEQ ID NO: 193 - protein, 25 aa, synthetic construct
GGGGSGGGGSGGGGSGGGGSGGGGS

SEQ ID NO: 194 - skipped

SEQ ID NO: 195 - protein, 22 aa, synthetic construct
MDMRVPAQLLGLLLLWLRGARC

SEQ ID NO: 196 - DNA, 66 bp, synthetic construct
  source 1..66: mol_type = other DNA
atggatatgagggtccccgcacaattgctcggtctccttctgctttggctccggggcgcgcggtgc

SEQ ID NO: 197 - protein, 22 aa, synthetic construct
MDMRVLAQLLGLLLLCFPGARC

SEQ ID NO: 198 - protein, 20 aa, synthetic construct
MEAPAQLLFLLLLWLPDTTG

SEQ ID NO: 199 - protein, 20 aa, synthetic construct
MVLQTQVFISLLLWISGAYG

SEQ ID NO: 200 - protein, 19 aa, synthetic construct
MLPSQLIGFLLLWVPASRG

SEQ ID NO: 201 - protein, 22 aa, synthetic construct
MDMRVPAQLLGLLLLWLSGARC

SEQ ID NO: 202 - protein, 15 aa, synthetic construct
EPKSCDKTHTCPPCP

SEQ ID NO: 203 - protein, 15 aa, synthetic construct
DKTHTCPPCPAPPVA

SEQ ID NO: 204 - protein, 12 aa, synthetic construct
ESKYGPPCPPCP

SEQ ID NO: 205 - protein, 12 aa, synthetic construct
ESKYGPPCPSCP

SEQ ID NO: 206 - protein, 49 aa, synthetic construct
KPTTTPAPRPPTPAPTIASQPLSLRPEACRPAAGGAVHTRGLDFACDIY

SEQ ID NO: 207 - protein, 39 aa, synthetic construct
IEVMYPPPYLDNERSNGTIIHVKGKHLCPSPLFPGPSKP

SEQ ID NO: 208 - protein, 47 aa, synthetic construct
TTTPAPRPPTPAPTIASQPLSLRPEACRPAAGGAVHTRGLDFACDIY

SEQ ID NO: 209 - protein, 45 aa, synthetic construct
TTTPAPRPPTPAPTIASQPLSLRPEACRPAAGGAVHTRGLDFACD

SEQ ID NO: 210 - protein, 21 aa, synthetic construct
LCYLLDGILFIYGVILTALFL

SEQ ID NO: 211 - protein, 22 aa, synthetic construct
MALIVLGGVAGLLLFIGLGIFF

SEQ ID NO: 212 - protein, 21 aa, synthetic construct
IYIWAPLAGTCGVLLLSLVIT

SEQ ID NO: 213 - protein, 24 aa, synthetic construct
IYIWAPLAGTCGVLLLSLVITLYC

SEQ ID NO: 214 - protein, 22 aa, synthetic construct
IWAPLAGTCGVLLLSLVITLYC

SEQ ID NO: 215 - protein, 19 aa, synthetic construct
IWAPLAGTCGVLLLSLVIT

SEQ ID NO: 216 - protein, 27 aa, synthetic construct
FWVLVVVGGVLACYSLLVTVAFIIFWV

SEQ ID NO: 217 - protein, 21 aa, synthetic construct
FWLPIGCAAFVVVCILGCILI

SEQ ID NO: 218 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 219 - protein, 42 aa, synthetic construct
KRGRKKLLYIFKQPFMRPVQTTQEEDGCSCRFPEEEEGGCEL

SEQ ID NO: 220 - protein, 37 aa, synthetic construct
KRGRKKLLYIFKQPFMRPVQTTQEEDGCSCRFPEEEE

SEQ ID NO: 221 - protein, 41 aa, synthetic construct
RSKRSRLLHSDYMNMTPRRPGPTRKHYQPYAPPRDFAAYRS

SEQ ID NO: 222 - protein, 42 aa, synthetic construct
ALYLLRRDQRLPPDAHKPPGGGSFRTPIQEEQADAHSTLAKI

SEQ ID NO: 223 - protein, 38 aa, synthetic construct
CWLTKKKYSSSVHDPNGEYMFMRAVNTAKKSRLTDVTL

SEQ ID NO: 224 - protein, 24 aa, synthetic construct
LCARPRRSPAQEDGKVYINMPGRG

SEQ ID NO: 225 - protein, 52 aa, synthetic construct
YFLGRLVPRGRGAAEAATRKQRITETESPYQELQGQRSDVYSDLNTQRPYYK

SEQ ID NO: 226 - protein, 48 aa, synthetic construct
QRRKYRSNKGESPVEPAEPCHYSCPREEEGSTIPIQEDYRKPEPACSP

SEQ ID NO: 227 - protein, 503 aa, synthetic construct
MLLLVTSLLLCELPHPAFLLIPDTSAQAAQVTLKESGAEVKKPGESLKISCKASGYSFTNYWIGWVRQMPGKGLEWMGIIYPGDSDTRYSPSFQGQVTISADKSISTAYLQWSSLKASDTAMYYCARQGGYYYGMDVWGQGTTVTVSSGGGGSGGGGSGGGGSVPVLTQPHSVSESPGKTVTISCTRSSGSIASNYVQWYQQRPGSSPTTVIYEDNQRPSGVPDRFSGSIDMSSNSASLTISGLRTEDEGDYYCQSYDSRNHVVFGGGTQLTVLGGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 228 - DNA, 1509 bp, synthetic construct
  source 1..1509: mol_type = other DNA
atgcttctgctcgtgacaagcctgctgctgtgcgagctgccccaccctgcctttctgctgatccctgacacctcggcccaggcggcccaggtcaccttgaaggagtctggggcagaggtgaaaaagccgggggagtctctgaaaatctcctgtaaggcttctggatacagctttaccaattactggatcggctgggtgcgccagatgcccggcaaaggcctggagtggatggggatcatctatcctggtgactctgataccagatacagcccgtccttccaaggccaggtcaccatctcagccgacaagtccatcagcactgcctacctgcagtggagcagcctgaaggcctcggacaccgccatgtattactgtgcgagacaggggggctactactacggtatggacgtctggggccaagggaccacggtcaccgtctcctcaggaggtggcgggtctggtggtggcggaagcggtggtggcggatccgtgcctgtgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacatgtcgtccaactctgcctccctcaccatctctggactgaggactgaggacgagggtgactattactgtcagtcttatgatagccgcaatcatgtggtattcggcggagggacccagctcaccgttttaggtggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 229 - protein, 501 aa, synthetic construct
MLLLVTSLLLCELPHPAFLLIPDTSAQAAQVQLQQSGAEVKKPGESLKISCKGSGYSFTNHWIAWVRQMPGKGLEWMGIIYPGDSDTRYSPSFQGQVTISADKSISTAYLQWSSLKASDTAMYYCARAQVGAFDIWGQGTMVTVSSGGGGSGGGGSGGGGSQSALTQPASVSGSPGQSITISCTRARDDVGGDNFVSWYQQHPGKAPKLMIYDVSNRPSGVSNRFSGSKSGNTASLTISGLQAEDEADYYCSSYTSSSTLPVFGGGTQLTVLGGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 230 - DNA, 1503 bp, synthetic construct
  source 1..1503: mol_type = other DNA
atgcttctgctcgtgacaagcctgctgctgtgcgagctgccccaccctgcctttctgctgatccctgacacctcggcccaggcggcccaggtacagctgcagcagtcaggagcagaggtgaaaaagcccggggagtctctgaagatctcctgtaagggttctggatacagctttaccaaccactggatcgcctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagagctcaagtcggggcttttgatatctggggccaagggacaatggtcaccgtctcttcaggaggtggcgggtctggtggtggcggaagcggtggtggcggatcccagtctgccctgactcagcctgcctccgtgtctgggtctcctggacagtcgatcaccatctcctgcactagagccagggatgacgttggtggtgacaactttgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactcttccggtgttcggcgggggcacccagctgaccgtcctcggtggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 231 - protein, 375 aa, synthetic construct
MLLLVTSLLLCELPHPAFLLIPDTSAQAAEVQLVESGGGVVQPGRSLRLSCAASGFTFSSYAMHWVRQAPGKGLEWVATIYQDGSESEYVDSVKGRFTVSRDNAKNTLYLQMDSLRAEDTATYYCVGRGSTSITPVWGLGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 232 - DNA, 1125 bp, synthetic construct
  source 1..1125: mol_type = other DNA
atgcttctgctcgtgacaagcctgctgctgtgcgagctgccccaccctgcctttctgctgatccctgacacctcggcccaggcggccgaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccagagacaacgccaagaacacgctgtatctgcaaatggacagtctgagagccgaggacacagccacgtattactgtgtgggacgcggttcgacttcaataacacctgtctggggcctaggaaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 233 - protein, 375 aa, synthetic construct
MLLLVTSLLLCELPHPAFLLIPDTSAQAAEVQLVESGGGVVQPGRSLRLSCAASGFTFSSYAMHWVRQAPGKGLEWVATIYQDGSESEYVDSVKGRFTVSRDDAKNTLYLQMDSLRAEDTATYYCVGRDSTSITPVWGLGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 234 - DNA, 1125 bp, synthetic construct
  source 1..1125: mol_type = other DNA
atgcttctgctcgtgacaagcctgctgctgtgcgagctgccccaccctgcctttctgctgatccctgacacctcggcccaggcggccgaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccagagacgacgccaagaacacgctgtatctgcaaatggacagtctgagagccgaggacacagccacgtattactgtgtgggacgagattcgacttcaataacacctgtctggggcctaggaaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 235 - protein, 375 aa, synthetic construct
MLLLVTSLLLCELPHPAFLLIPDTSAQAAEVQLVESGGGMVQPGRSLRLSCAASGFTFSSYAMHWIRQAPGKGLEWVATIYQDGSESEYVDSVKGRFAVSRDNAKNTLYLQMDSLRAEDTATYYCVGRASASITPVWGLGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 236 - DNA, 1125 bp, synthetic construct
  source 1..1125: mol_type = other DNA
atgcttctgctcgtgacaagcctgctgctgtgcgagctgccccaccctgcctttctgctgatccctgacacctcggcccaggcggccgaggtgcagctggtggagtccgggggaggcatggtccagcctgggaggtccctgagactctcctgtgcagcctctggattcaccttcagtagctatgctatgcactggatccgccaggctccagggaaaggcctggagtgggtggccaccatataccaagatggaagtgagagtgaatatgtagactctgtgaagggccgattcgccgtctccagagacaacgccaagaacacgctgtatctgcaaatggacagtctgagagccgaggacacagccacgtattactgtgtgggacgcgcttcggcttcaataacacctgtctggggcctaggaaccctggtcaccgtctcctcaggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 237 - protein, 372 aa, synthetic construct
MDMRVPAQLLGLLLLWLRGARCEVQLVESGGGLVQPGGSLRLSCTVSGFTFSSYAVSWVRQAPGQGLEWVSAIGAGGGTYYADSVKGRFTISRDNSKNTLYLQMNSLRAEDTAVYYCAKDGADWGQGTLVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 238 - DNA, 1119 bp, synthetic construct
  source 1..1119: mol_type = other DNA
atggatatgagggtccccgcacaattgctcggtctccttctgctttggctccggggcgcgcggtgcgaggtgcagctggtggagtctgggggaggcttggtacagcctggagggtccctgagactctcctgtaccgtctctggattcacctttagcagctatgccgtgagctgggtccgccaggctccaggacaagggcttgagtgggtatcagctattggtgctggtggtggcacatactacgcagactctgtgaagggccgattcaccatttccagagacaattccaagaacacgctgtatctgcaaatgaacagcctgagagccgaggacacagccgtgtattactgtgcgaaagacggggccgactggggccagggaaccctggtcaccgtctcctcaagtgcggccgcggagtctaaatacggtcctccatgtcccagctgtcccttttgggttctcgtcgtagtcggaggagtattggcctgctattctcttctcgtaactgtcgcttttataattttctgggtacgaagtaagcgaagtaggttgcttcatagtgattacatgaatatgacaccacgacggcccggccccactcggaagcactaccaaccgtatgctccccctcgagactttgccgcgtatcgatctaaacggggccgaaaaaagttgctgtacatctttaaacaaccatttatgagaccagttcaaacgacccaggaagaagatggatgctcatgccgcttcccagaagaggaagaaggggggggtggttcacgggttaaattctcaaggtctgctgatgcccccgcctatcaacagggacagaatcagttgtacaacgagctcaatcttggaaggcgcgaagagtatgatgtgttggataaaagaagaggtcgcgaccctgaaatggggggcaagcctcagagacgcaaaaaccctcaggagggactctataatgagttgcagaaggataagatggccgaagcatactcagagataggtatgaaaggggagcgacgaagagggaagggacatgatgggctgtaccaagggctcagtactgcgacgaaggacacatatgacgcactccatatgcaggctttgccacctcgc

SEQ ID NO: 239 - protein, 372 aa, synthetic construct
MDMRVPAQLLGLLLLWLRGARCEVQLVESGGGLVQPGGSLRLSCTVSGFTFSSYAVSWVRQAPGQGLEWVSAIGAGGGTYYADSVKGRFTISRDNSKNTLYLQMNSLRAEDTAVYYCAKDGVDWGQGTLVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 240 - DNA, 1119 bp, synthetic construct
  source 1..1119: mol_type = other DNA
atggatatgagggtccccgcacaattgctcggtctccttctgctttggctccggggcgcgcggtgcgaggtgcagctggtggagtctgggggaggcttggtacagcctggagggtccctgagactctcctgtaccgtctctggattcacctttagcagctatgccgtgagctgggtccgccaggctccaggacaagggcttgagtgggtatcagctattggtgctggtggtggcacatactacgcagactctgtgaagggccgattcaccatttccagagacaattccaagaacacgctgtatctgcaaatgaacagcctgagagccgaggacacagccgtgtattactgtgcgaaagacggggtcgactggggccagggaaccctggtcaccgtctcctcaagtgcggccgcggagtctaaatacggtcctccatgtcccagctgtcccttttgggttctcgtcgtagtcggaggagtattggcctgctattctcttctcgtaactgtcgcttttataattttctgggtacgaagtaagcgaagtaggttgcttcatagtgattacatgaatatgacaccacgacggcccggccccactcggaagcactaccaaccgtatgctccccctcgagactttgccgcgtatcgatctaaacggggccgaaaaaagttgctgtacatctttaaacaaccatttatgagaccagttcaaacgacccaggaagaagatggatgctcatgccgcttcccagaagaggaagaaggggggggtggttcacgggttaaattctcaaggtctgctgatgcccccgcctatcaacagggacagaatcagttgtacaacgagctcaatcttggaaggcgcgaagagtatgatgtgttggataaaagaagaggtcgcgaccctgaaatggggggcaagcctcagagacgcaaaaaccctcaggagggactctataatgagttgcagaaggataagatggccgaagcatactcagagataggtatgaaaggggagcgacgaagagggaagggacatgatgggctgtaccaagggctcagtactgcgacgaaggacacatatgacgcactccatatgcaggctttgccacctcgc

SEQ ID NO: 241 - protein, 372 aa, synthetic construct
MDMRVPAQLLGLLLLWLRGARCEVQLVESGGGLVQPGGSLRLSCTVSGFTFSSYAVSWVRQAPGQGLEWVSSIGAGGGTYYADSVKGRFTISRDNSKNTLYLQMNSLRAEDTAVYYCAKDGADWGQGTLVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 242 - DNA, 1119 bp, synthetic construct
  source 1..1119: mol_type = other DNA
atggatatgagggtccccgcacaattgctcggtctccttctgctttggctccggggcgcgcggtgcgaggtgcagctggtggagtctgggggaggcttggtacagcccggagggtccctgagactctcctgtaccgtctctggattcacctttagcagctatgccgtgagctgggtccgccaggctccaggacaagggcttgagtgggtatcatctattggtgctggtggtggcacatactacgcagactctgtgaagggccgattcaccatttccagagacaattccaagaacacgctgtatctgcaaatgaacagcctgagagccgaggacacagccgtgtattactgtgcgaaagacggggccgactggggccagggaaccctggtcaccgtctcctcaagtgcggccgcggagtctaaatacggtcctccatgtcccagctgtcccttttgggttctcgtcgtagtcggaggagtattggcctgctattctcttctcgtaactgtcgcttttataattttctgggtacgaagtaagcgaagtaggttgcttcatagtgattacatgaatatgacaccacgacggcccggccccactcggaagcactaccaaccgtatgctccccctcgagactttgccgcgtatcgatctaaacggggccgaaaaaagttgctgtacatctttaaacaaccatttatgagaccagttcaaacgacccaggaagaagatggatgctcatgccgcttcccagaagaggaagaaggggggggtggttcacgggttaaattctcaaggtctgctgatgcccccgcctatcaacagggacagaatcagttgtacaacgagctcaatcttggaaggcgcgaagagtatgatgtgttggataaaagaagaggtcgcgaccctgaaatggggggcaagcctcagagacgcaaaaaccctcaggagggactctataatgagttgcagaaggataagatggccgaagcatactcagagataggtatgaaaggggagcgacgaagagggaagggacatgatgggctgtaccaagggctcagtactgcgacgaaggacacatatgacgcactccatatgcaggctttgccacctcgc

SEQ ID NO: 243 - protein, 369 aa, synthetic construct
MLLLVTSLLLCELPHPAFLLIPDTSAQAAEVQLVESGGGLVQPGGSLRLSCTVSGFTFDDYAVSWVRQAPGQGLEWVSSIGAGGGTYYADSVKGRFTISRDDSKDTLYLQMNSLRAEDTAVYYCAKDGVDWGQGTLVTVSSGQAGRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 244 - DNA, 1110 bp, synthetic construct
  source 1..1110: mol_type = other DNA
atgcttctgctcgtgacaagcctgctgctgtgcgagctgccccaccctgcctttctgctgatccctgacacctcggcccaggcggccgaggtgcagctggtggagtctgggggaggcttggtacagcccggagggtccctgagactctcctgtaccgtctctggattcacctttgacgactatgccgtgagctgggtccgccaggctccaggacaagggcttgagtgggtatcatctattggtgctggtggtggcacatactacgcagactctgtgaagggccgattcaccatttccagagacgattccaaggacacgctgtatctgcaaatgaacagcctgagagccgaggacacagccgtgtattactgtgcgaaagacggggtcgactggggccagggaaccctggtcaccgtctcctcaagtggccaggccggccgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 245 - protein, 378 aa, synthetic construct
MDMRVPAQLLGLLLLWLRGARCEVQMVESGGGVVQPGRSLRLSCAASGFTFSSYAMHWVRQAPGKGLEWVATIYQDGSESEYVDSVKGRFTVSIDNAKNTLYLQMDSLRAEDTATYYCVGRDSTSITPVWGLGTLVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 246 - DNA, 1137 bp, synthetic construct
  source 1..1137: mol_type = other DNA
atggatatgagggtccccgcacaattgctcggtctccttctgctttggctccggggcgcgcggtgcgaggtgcagatggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccatagacaacgccaagaacacgctgtatctgcaaatggacagtctgagagccgaggacacagccacgtattactgtgtgggacgcgattcgacttcaataacacctgtctggggcctaggaaccctggtcaccgtctcctcaagtgcggccgcggagtctaaatacggtcctccatgtcccagctgtcccttttgggttctcgtcgtagtcggaggagtattggcctgctattctcttctcgtaactgtcgcttttataattttctgggtacgaagtaagcgaagtaggttgcttcatagtgattacatgaatatgacaccacgacggcccggccccactcggaagcactaccaaccgtatgctccccctcgagactttgccgcgtatcgatctaaacggggccgaaaaaagttgctgtacatctttaaacaaccatttatgagaccagttcaaacgacccaggaagaagatggatgctcatgccgcttcccagaagaggaagaaggggggggtggttcacgggttaaattctcaaggtctgctgatgcccccgcctatcaacagggacagaatcagttgtacaacgagctcaatcttggaaggcgcgaagagtatgatgtgttggataaaagaagaggtcgcgaccctgaaatggggggcaagcctcagagacgcaaaaaccctcaggagggactctataatgagttgcagaaggataagatggccgaagcatactcagagataggtatgaaaggggagcgacgaagagggaagggacatgatgggctgtaccaagggctcagtactgcgacgaaggacacatatgacgcactccatatgcaggctttgccacctcgc

SEQ ID NO: 247 - protein, 378 aa, synthetic construct
MDMRVPAQLLGLLLLWLRGARCEVQLVESGGGVVQPGRSLRLSCAASGFTFSSYAMHWVRQAPGKGLEWVATIYQDGSESEYVDSVKGRFTVSIDNAKNTLYLQMDSLRAEDTATYYCVGRASTSITPVWGLGTLVTVSSAAAESKYGPPCPSCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 248 - DNA, 1137 bp, synthetic construct
  source 1..1137: mol_type = other DNA
atggatatgagggtccccgcacaattgctcggtctccttctgctttggctccggggcgcgcggtgcgaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccatagacaacgccaagaacacgctgtatctgcaaatggacagtctgagagccgaggacacagccacgtattactgtgtgggacgcgcttcgacttcaataacacctgtctggggcctaggaaccctggtcaccgtctcctcaagtgcggccgcggagtctaaatacggtcctccatgtcccagctgtcccttttgggttctcgtcgtagtcggaggagtattggcctgctattctcttctcgtaactgtcgcttttataattttctgggtacgaagtaagcgaagtaggttgcttcatagtgattacatgaatatgacaccacgacggcccggccccactcggaagcactaccaaccgtatgctccccctcgagactttgccgcgtatcgatctaaacggggccgaaaaaagttgctgtacatctttaaacaaccatttatgagaccagttcaaacgacccaggaagaagatggatgctcatgccgcttcccagaagaggaagaaggggggggtggttcacgggttaaattctcaaggtctgctgatgcccccgcctatcaacagggacagaatcagttgtacaacgagctcaatcttggaaggcgcgaagagtatgatgtgttggataaaagaagaggtcgcgaccctgaaatggggggcaagcctcagagacgcaaaaaccctcaggagggactctataatgagttgcagaaggataagatggccgaagcatactcagagataggtatgaaaggggagcgacgaagagggaagggacatgatgggctgtaccaagggctcagtactgcgacgaaggacacatatgacgcactccatatgcaggctttgccacctcgc

SEQ ID NO: 249 - protein, 271 aa, synthetic construct
GGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 250 - DNA, 813 bp, synthetic construct
  source 1..813: mol_type = other DNA
ggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactagg

SEQ ID NO: 251 - protein, 119 aa, synthetic construct [sequence omitted]

SEQ ID NO: 252 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 253 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 254 - protein, 108 aa, synthetic construct
MDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 255 - protein, 117 aa, synthetic construct
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 256 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 257 - protein, 125 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 258 - protein, 107 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 259 - protein, 119 aa, synthetic construct
EVQLVESGGGLVKPGGSLRLSCAASGFTFSSYAMSWVRQAPGKGLEWVSEISSGGSYTYYADSVKGRFTISRDNAKNSLYLQMNSLRAEDTAVYYCARHGDYPRFFDVWGQGTTVTVSS

SEQ ID NO: 260 - protein, 107 aa, synthetic construct
EIVLTQSPATLSLSPGERATLSCSASSSVSSYIYWYQQKPGQAPRLLIYLTSNLASGIPARFSGSGSGTDFTLTISSLEPEDFAVYYCQQWSGNPYTFGQGTKLEIK

SEQ ID NO: 261 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 262 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 263 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTLVTVSS

SEQ ID NO: 264 - protein, 114 aa, synthetic construct
QVQLVQSGAEVKKPGASVKVSCKASGHTFTSYFMHWVRQAPGQGLEWMGIINPSDDYANYAQKFQGRVTMTRDTSTSTVYMELSSLRSEDTAVYYCATAIFDYWGQGTPVTVSS

SEQ ID NO: 265 - protein, 107 aa, synthetic construct [sequence omitted]

SEQ ID NO: 266 - protein, 114 aa, synthetic construct
EIQLQQSGAELVKPGASVKLSCTASGFNIKDTYFHWVKQRPEQGLEWIGRIDPANGNTKYDPKFHDKATIIADISSNTAYLQFSSLTSEDTAVYYCAANRYGYWGQGTTLTVSS

SEQ ID NO: 267 - protein, 112 aa, synthetic construct
DIVMTQAAPSIPVTPGESVSISCRSSKSLLYINGNTHLFWFLQRPGQSPQLLIYRMSNLASGVPDRFSGSGSGTAFTLRISRVEAEDVGVYYCMQHLEYPFTFGSGTKLEIK

SEQ ID NO: 268 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 269 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 270 - protein, 113 aa, synthetic construct
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 271 - protein, 501 aa, synthetic construct
QVTLKESGAEVKKPGESLKISCKASGYSFTNYWIGWVRQMPGKGLEWMGIIYPGDSDTRYSPSFQGQVTISADKSISTAYLQWSSLKASDTAMYYCARQGGYYYGMDVWGQGTTVTVSSGGGGSGGSGSGGGGSVPVLTQPHSVSESPGKTVTISCTRSSGSIASNYVQWYQQRPGSSPTTVIYEDNQRPSGVPDRFSGSIDMSSNSASLTISGLRTEDEGDYYCQSYDSRNHVVFGGGTQLTVLGGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 272 - protein, 499 aa, synthetic construct
QVQLQQSGAEVKKPGESLKISCKGSGYSFTNHWIAWVRQMPGKGLEWMGIIYPGDSDTRYSPSFQGQVTISADKSISTAYLQWSSLKASDTAMYYCARAQVGAFDIWGQGTMVTVSSGGGGSGGSGSSGGGSQSALTQPASVSGSPGQSITISCTRARDDVGGDNFVSWYQQHPGKAPKLMIYDVSNRPSGVSNRFSGSKSGNTASLTISGLQAEDEADYYCSSYTSSSTLPVFGGGTQLTVLGGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSVEGGSGGSGGSGGSGGVDDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 273 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 274 - protein, 707 aa, Homo sapiens
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 275 - DNA, 45 bp, synthetic construct
  source 1..45: mol_type = other DNA
ggaggtggcgggtctggtggaggcggaagcggtggtggcggatcc

SEQ ID NO: 276 - protein, 8 aa, synthetic construct
EPKSCDKT

SEQ ID NO: 277 - protein, 8 aa, synthetic construct
GYTFTGYY

SEQ ID NO: 278 - protein, 8 aa, synthetic construct
GYSFTSYW

SEQ ID NO: 279 - protein, 8 aa, synthetic construct
GYTFTIYG

SEQ ID NO: 280 - protein, 8 aa, synthetic construct
GYSFTTYG

SEQ ID NO: 281 - protein, 8 aa, synthetic construct
GYTFTSYA

SEQ ID NO: 282 - protein, 8 aa, synthetic construct
GYTFTSYD

SEQ ID NO: 283 - protein, 8 aa, synthetic construct
GYTFTSYG

SEQ ID NO: 284 - protein, 8 aa, synthetic construct
GYTLTELS

SEQ ID NO: 285 - protein, 8 aa, synthetic construct
GYTFTYRY

SEQ ID NO: 286 - protein, 8 aa, synthetic construct
GYTFTCCS

SEQ ID NO: 287 - protein, 7 aa, synthetic construct
IYWNDDK

SEQ ID NO: 288 - protein, 7 aa, synthetic construct
IYSGGST

SEQ ID NO: 289 - protein, 8 aa, synthetic construct
IYSGGSST

SEQ ID NO: 290 - protein, 8 aa, synthetic construct
IYPGNSDT

SEQ ID NO: 291 - protein, 8 aa, synthetic construct
IDPSDSYT

SEQ ID NO: 292 - protein, 8 aa, synthetic construct
IYSGGSST

SEQ ID NO: 293 - protein, 8 aa, synthetic construct
INSDGSST

SEQ ID NO: 294 - protein, 8 aa, synthetic construct
ISSNGGST

SEQ ID NO: 295 - protein, 8 aa, synthetic construct
IKDDGSQI

SEQ ID NO: 296 - protein, 8 aa, synthetic construct
IQCDGSQI

SEQ ID NO: 297 - protein, 12 aa, synthetic construct
ARQGGFYYGMDV

SEQ ID NO: 298 - protein, 12 aa, synthetic construct
ARQGGYFYGMDV

SEQ ID NO: 299 - protein, 12 aa, synthetic construct
ARQRGYYYGMDV

SEQ ID NO: 300 - protein, 12 aa, synthetic construct
ARQGRYYFGMDV

SEQ ID NO: 301 - protein, 12 aa, synthetic construct
ARQSGYYYGMDV

SEQ ID NO: 302 - protein, 12 aa, synthetic construct
ARQGSYYYGMDV

SEQ ID NO: 303 - protein, 12 aa, synthetic construct
ARQGGYYGGMDV

SEQ ID NO: 304 - protein, 12 aa, synthetic construct
ARQGGYAYGMDV

SEQ ID NO: 305 - protein, 12 aa, synthetic construct
ARQRGAYYGMDV

SEQ ID NO: 306 - protein, 12 aa, synthetic construct
ARQGGAYYRMDV

SEQ ID NO: 307 - protein, 7 aa, synthetic construct
SGYSNYK

SEQ ID NO: 308 - protein, 9 aa, synthetic construct
SGSVSTSYY

SEQ ID NO: 309 - protein, 9 aa, synthetic construct
SGINVGTYR

SEQ ID NO: 310 - protein, 6 aa, synthetic construct
SLRSYY

SEQ ID NO: 311 - protein, 9 aa, synthetic construct
SDINVGSYN

SEQ ID NO: 312 - protein, 7 aa, synthetic construct
SGHSSYA

SEQ ID NO: 313 - protein, 7 aa, synthetic construct
SGHSSYI

SEQ ID NO: 314 - protein, 7 aa, synthetic construct
SEHSTYT

SEQ ID NO: 315 - protein, 9 aa, synthetic construct
SDLSVGGKN

SEQ ID NO: 316 - protein, 9 aa, synthetic construct
SGINLGSYR

SEQ ID NO: 317-326 - skipped (10 entries)

SEQ ID NO: 327 - protein, 10 aa, synthetic construct
QSGDSRNHVV

SEQ ID NO: 328 - protein, 10 aa, synthetic construct
QQYDSRNHVV

SEQ ID NO: 329 - protein, 10 aa, synthetic construct
QKYDSRNHVV

SEQ ID NO: 330 - protein, 10 aa, synthetic construct
QSYYSRNHVV

SEQ ID NO: 331 - protein, 10 aa, synthetic construct
QSYDSRNQVV

SEQ ID NO: 332 - protein, 10 aa, synthetic construct
QSYGSRNHVV

SEQ ID NO: 333 - protein, 10 aa, synthetic construct
QSYASRNHVV

SEQ ID NO: 334 - protein, 10 aa, synthetic construct
QYSDSRNHVV

SEQ ID NO: 335 - protein, 11 aa, synthetic construct
QSYDDYRNHVV

SEQ ID NO: 336 - protein, 10 aa, synthetic construct
QSYDSRYHVV

SEQ ID NO: 337 - protein, 8 aa, synthetic construct
GYSFTTYG

SEQ ID NO: 338 - protein, 8 aa, synthetic construct
GYTFTIYG

SEQ ID NO: 339 - protein, 8 aa, synthetic construct
GYTFTSYA

SEQ ID NO: 340 - protein, 8 aa, synthetic construct
GYSFTSYW

SEQ ID NO: 341 - protein, 9 aa, synthetic construct
GYSISSGYY

SEQ ID NO: 342 - protein, 9 aa, synthetic construct
GYSISSSNW

SEQ ID NO: 343 - protein, 9 aa, synthetic construct
GGSISSSNW

SEQ ID NO: 344 - protein, 10 aa, synthetic construct
GGSISSGGYS

SEQ ID NO: 345 - protein, 10 aa, synthetic construct
GGSISSSSYY

SEQ ID NO: 346 - protein, 8 aa, synthetic construct
GGSISSYY

SEQ ID NO: 347 - protein, 7 aa, synthetic construct
IYWNDDK

SEQ ID NO: 348 - protein, 7 aa, synthetic construct
IYSGGST

SEQ ID NO: 349 - protein, 8 aa, synthetic construct
IYSGGSST

SEQ ID NO: 350 - protein, 8 aa, synthetic construct
IYPGNSDT

SEQ ID NO: 351 - protein, 8 aa, synthetic construct
IDPSDSYT

SEQ ID NO: 352 - protein, 8 aa, synthetic construct
IYSGGSST

SEQ ID NO: 353 - protein, 8 aa, synthetic construct
INSDGSST

SEQ ID NO: 354 - protein, 8 aa, synthetic construct
ISSNGGST

SEQ ID NO: 355 - protein, 8 aa, synthetic construct
IKDDGSQI

SEQ ID NO: 356 - protein, 8 aa, synthetic construct
IQCDGSQI

SEQ ID NO: 357 - protein, 10 aa, synthetic construct
ARALVGAFDI

SEQ ID NO: 358 - protein, 10 aa, synthetic construct
ARARVGAFDI

SEQ ID NO: 359 - protein, 10 aa, synthetic construct
ARGQVGAFDI

SEQ ID NO: 360 - protein, 10 aa, synthetic construct
ARAQVGSFDI

SEQ ID NO: 361 - protein, 10 aa, synthetic construct
ARAQVGAFLI

SEQ ID NO: 362 - protein, 10 aa, synthetic construct
ARAQTGAFDI

SEQ ID NO: 363 - protein, 10 aa, synthetic construct
ARSQVGAFDI

SEQ ID NO: 364 - protein, 10 aa, synthetic construct
ARAQVWAFDI

SEQ ID NO: 365 - protein, 10 aa, synthetic construct
ARAQVGNFDI

SEQ ID NO: 366 - protein, 10 aa, synthetic construct
ARAQVGLFDI

SEQ ID NO: 367 - protein, 9 aa, synthetic construct
SSDVGGYNY

SEQ ID NO: 368 - protein, 9 aa, synthetic construct
SSDVGSYNR

SEQ ID NO: 369 - protein, 9 aa, synthetic construct
SSDVGSYNL

SEQ ID NO: 370 - protein, 9 aa, synthetic construct
SSDVGDYDH

SEQ ID NO: 371 - protein, 9 aa, synthetic construct
SSDIGGYDL

SEQ ID NO: 372 - protein, 9 aa, synthetic construct
SSDVGSYDY

SEQ ID NO: 373 - protein, 9 aa, synthetic construct
SSNTGTGYN

SEQ ID NO: 374 - protein, 9 aa, synthetic construct
SDINVGSYN

SEQ ID NO: 375 - protein, 9 aa, synthetic construct
SGINVGTYR

SEQ ID NO: 376 - protein, 9 aa, synthetic construct
SGINLGSYR

SEQ ID NO: 377-386 - skipped (10 entries)

SEQ ID NO: 387 - protein, 11 aa, synthetic construct
SRYTSSSTLPV

SEQ ID NO: 388 - protein, 11 aa, synthetic construct
SDYTSSSTLPV

SEQ ID NO: 389 - protein, 11 aa, synthetic construct
SSYTSSNTLPV

SEQ ID NO: 390 - protein, 11 aa, synthetic construct
SSYTGSSTLPV

SEQ ID NO: 391 - protein, 11 aa, synthetic construct
SSYTSDDTLPV

SEQ ID NO: 392 - protein, 11 aa, synthetic construct
SSYTSGSTLPV

SEQ ID NO: 393 - protein, 11 aa, synthetic construct
SSYTSSSTWPV

SEQ ID NO: 394 - protein, 11 aa, synthetic construct
SSYLSSSTLPV

SEQ ID NO: 395 - protein, 11 aa, synthetic construct
SSYTSSSTLPV

SEQ ID NO: 396 - protein, 11 aa, synthetic construct
SSYTSSSTLPV

SEQ ID NO: 397 - protein, 8 aa, synthetic construct
GFTFSSYW

SEQ ID NO: 398 - protein, 8 aa, synthetic construct
GFTFDDYA

SEQ ID NO: 399 - protein, 8 aa, synthetic construct
GFTFSDYY

SEQ ID NO: 400 - protein, 8 aa, synthetic construct
GFTFSSYD

SEQ ID NO: 401 - protein, 8 aa, synthetic construct
GFTFSNAW

SEQ ID NO: 402 - protein, 8 aa, synthetic construct
GFTFSNSD

SEQ ID NO: 403 - protein, 8 aa, synthetic construct
GFTFSNSD

SEQ ID NO: 404 - protein, 8 aa, synthetic construct
GFTFDDYG

SEQ ID NO: 405 - protein, 8 aa, synthetic construct
GFTFSSYS

SEQ ID NO: 406 - protein, 8 aa, synthetic construct
GFTFSSYG

SEQ ID NO: 407 - protein, 8 aa, synthetic construct
IKQDGSEK

SEQ ID NO: 408 - protein, 8 aa, synthetic construct
ISWNSGSI

SEQ ID NO: 409 - protein, 8 aa, synthetic construct
ISSSGSTI

SEQ ID NO: 410 - protein, 7 aa, synthetic construct
IGTAGDT

SEQ ID NO: 411 - protein, 10 aa, synthetic construct
IKSKTDGGTT

SEQ ID NO: 412 - protein, 8 aa, synthetic construct
VSWNGSRT

SEQ ID NO: 413 - protein, 8 aa, synthetic construct
INWNGGST

SEQ ID NO: 414 - protein, 8 aa, synthetic construct
ISSSSSYI

SEQ ID NO: 415 - protein, 8 aa, synthetic construct
ISGSGGST

SEQ ID NO: 416 - protein, 8 aa, synthetic construct
IQCDGSQI

SEQ ID NO: 417 - protein, 11 aa, synthetic construct
VGRSSTSITPV

SEQ ID NO: 418 - protein, 11 aa, synthetic construct
VGRNSTSITPV

SEQ ID NO: 419 - protein, 11 aa, synthetic construct
VGRYSTSITPV

SEQ ID NO: 420 - protein, 11 aa, synthetic construct
VGRQSTSITPV

SEQ ID NO: 421 - protein, 11 aa, synthetic construct
VGRGSLSITPV

SEQ ID NO: 422 - protein, 11 aa, synthetic construct
VGRGSCSITPV

SEQ ID NO: 423 - protein, 11 aa, synthetic construct
VGRGSISITPV

SEQ ID NO: 424 - protein, 11 aa, synthetic construct
VGRGSTSILPV

SEQ ID NO: 425 - protein, 11 aa, synthetic construct
VGRGSTSLTPV

SEQ ID NO: 426 - protein, 11 aa, synthetic construct
VGRGSNSITPV

SEQ ID NO: 427 - protein, 8 aa, synthetic construct
GFTFSSYW

SEQ ID NO: 428 - protein, 8 aa, synthetic construct
GFTFDDYA

SEQ ID NO: 429 - protein, 8 aa, synthetic construct
GFTFSDYY

SEQ ID NO: 430 - protein, 8 aa, synthetic construct
GFTFSSYD

SEQ ID NO: 431 - protein, 8 aa, synthetic construct
GFTFSNAW

SEQ ID NO: 432 - protein, 8 aa, synthetic construct
GFTFSNSD

SEQ ID NO: 433 - protein, 8 aa, synthetic construct
GFTFSNSD

SEQ ID NO: 434 - protein, 8 aa, synthetic construct
GFTFDDYG

SEQ ID NO: 435 - protein, 8 aa, synthetic construct
GFTFSSYS

SEQ ID NO: 436 - protein, 8 aa, synthetic construct
GFTFSSYG

SEQ ID NO: 437 - protein, 8 aa, synthetic construct
IKQDGSEK

SEQ ID NO: 438 - protein, 8 aa, synthetic construct
ISWNSGSI

SEQ ID NO: 439 - protein, 8 aa, synthetic construct
ISSSGSTI

SEQ ID NO: 440 - protein, 7 aa, synthetic construct
IGTAGDT

SEQ ID NO: 441 - protein, 10 aa, synthetic construct
IKSKTDGGTT

SEQ ID NO: 442 - protein, 8 aa, synthetic construct
VSWNGSRT

SEQ ID NO: 443 - protein, 8 aa, synthetic construct
INWNGGST

SEQ ID NO: 444 - protein, 8 aa, synthetic construct
ISSSSSYI

SEQ ID NO: 445 - protein, 8 aa, synthetic construct
ISGSGGST

SEQ ID NO: 446 - protein, 8 aa, synthetic construct
IQCDGSQI

SEQ ID NO: 447 - protein, 11 aa, synthetic construct
VGRSSTSITPV

SEQ ID NO: 448 - protein, 11 aa, synthetic construct
VGRNSTSITPV

SEQ ID NO: 449 - protein, 11 aa, synthetic construct
VGRYSTSITPV

SEQ ID NO: 450 - protein, 11 aa, synthetic construct
VGRQSTSITPV

SEQ ID NO: 451 - protein, 11 aa, synthetic construct
VGRGSLSITPV

SEQ ID NO: 452 - protein, 11 aa, synthetic construct
VGRGSCSITPV

SEQ ID NO: 453 - protein, 11 aa, synthetic construct
VGRGSISITPV

SEQ ID NO: 454 - protein, 11 aa, synthetic construct
VGRGSTSILPV

SEQ ID NO: 455 - protein, 11 aa, synthetic construct
VGRGSTSLTPV

SEQ ID NO: 456 - protein, 11 aa, synthetic construct
VGRGSNSITPV

SEQ ID NO: 457 - protein, 8 aa, synthetic construct
GFTFSSYW

SEQ ID NO: 458 - protein, 8 aa, synthetic construct
GFTFDDYA

SEQ ID NO: 459 - protein, 8 aa, synthetic construct
GFTFSDYY

SEQ ID NO: 460 - protein, 8 aa, synthetic construct
GFTFSSYD

SEQ ID NO: 461 - protein, 8 aa, synthetic construct
GFTFSNAW

SEQ ID NO: 462 - protein, 8 aa, synthetic construct
GFTFSNSD

SEQ ID NO: 463 - protein, 8 aa, synthetic construct
GFTFSNSD

SEQ ID NO: 464 - protein, 8 aa, synthetic construct
GFTFDDYG

SEQ ID NO: 465 - protein, 8 aa, synthetic construct
GFTFSSYS

SEQ ID NO: 466 - protein, 8 aa, synthetic construct
GFTFSSYG

SEQ ID NO: 467 - protein, 8 aa, synthetic construct
IKQDGSEK

SEQ ID NO: 468 - protein, 8 aa, synthetic construct
ISWNSGSI

SEQ ID NO: 469 - protein, 8 aa, synthetic construct
ISSSGSTI

SEQ ID NO: 470 - protein, 7 aa, synthetic construct
IGTAGDT

SEQ ID NO: 471 - protein, 10 aa, synthetic construct
IKSKTDGGTT

SEQ ID NO: 472 - protein, 8 aa, synthetic construct
VSWNGSRT

SEQ ID NO: 473 - protein, 8 aa, synthetic construct
INWNGGST

SEQ ID NO: 474 - protein, 8 aa, synthetic construct
ISSSSSYI

SEQ ID NO: 475 - protein, 8 aa, synthetic construct
ISGSGGST

SEQ ID NO: 476 - protein, 8 aa, synthetic construct
IQCDGSQI

SEQ ID NO: 477 - protein, 11 aa, synthetic construct
VGRSSTSITPV

SEQ ID NO: 478 - protein, 11 aa, synthetic construct
VGRNSTSITPV

SEQ ID NO: 479 - protein, 11 aa, synthetic construct
VGRYSTSITPV

SEQ ID NO: 480 - protein, 11 aa, synthetic construct
VGRQSTSITPV

SEQ ID NO: 481 - protein, 11 aa, synthetic construct
VGRGSLSITPV

SEQ ID NO: 482 - protein, 11 aa, synthetic construct
VGRGSCSITPV

SEQ ID NO: 483 - protein, 11 aa, synthetic construct
VGRGSISITPV

SEQ ID NO: 484 - protein, 11 aa, synthetic construct
VGRGSTSILPV

SEQ ID NO: 485 - protein, 11 aa, synthetic construct
VGRGSTSLTPV

SEQ ID NO: 486 - protein, 11 aa, synthetic construct
VGRGSNSITPV

SEQ ID NO: 487 - protein, 8 aa, synthetic construct
GFTFSSYW

SEQ ID NO: 488 - protein, 8 aa, synthetic construct
GFTFDDYA

SEQ ID NO: 489 - protein, 8 aa, synthetic construct
GFTFSDYY

SEQ ID NO: 490 - protein, 8 aa, synthetic construct
GFTFSSYD

SEQ ID NO: 491 - protein, 8 aa, synthetic construct
GFTFSNAW

SEQ ID NO: 492 - protein, 8 aa, synthetic construct
GFTFSNSD

SEQ ID NO: 493 - protein, 8 aa, synthetic construct
GFTFSNSD

SEQ ID NO: 494 - protein, 8 aa, synthetic construct
GFTFDDYG

SEQ ID NO: 495 - protein, 8 aa, synthetic construct
GFTFSSYS

SEQ ID NO: 496 - protein, 8 aa, synthetic construct
GFTFSSYG

SEQ ID NO: 497 - protein, 10 aa, synthetic construct
IKSKTDGGTT

SEQ ID NO: 498 - protein, 8 aa, synthetic construct
INWNGGST

SEQ ID NO: 499 - protein, 8 aa, synthetic construct
ISGSGGST

SEQ ID NO: 500 - protein, 7 aa, synthetic construct
IGTAGDT

SEQ ID NO: 501 - protein, 8 aa, synthetic construct
VNPNGGST

SEQ ID NO: 502 - protein, 8 aa, synthetic construct
INSDGSST

SEQ ID NO: 503 - protein, 8 aa, synthetic construct
IYSGGSST

SEQ ID NO: 504 - protein, 7 aa, synthetic construct
IYHSGST

SEQ ID NO: 505 - protein, 8 aa, synthetic construct
INPSGGST

SEQ ID NO: 506 - protein, 8 aa, synthetic construct
INPNSGGT

SEQ ID NO: 507 - protein, 6 aa, synthetic construct
ARDGAD

SEQ ID NO: 508 - protein, 6 aa, synthetic construct
AKNGAD

SEQ ID NO: 509 - protein, 6 aa, synthetic construct
AKDGKD

SEQ ID NO: 510 - protein, 6 aa, synthetic construct
AKDGND

SEQ ID NO: 511 - protein, 6 aa, synthetic construct
ARDGND

SEQ ID NO: 512 - protein, 6 aa, synthetic construct
ARDGKD

SEQ ID NO: 513 - protein, 6 aa, synthetic construct
AKNGAD

SEQ ID NO: 514 - protein, 6 aa, synthetic construct
AKDGSD

SEQ ID NO: 515 - protein, 6 aa, synthetic construct
AKDGTD

SEQ ID NO: 516 - protein, 6 aa, synthetic construct
ARDSAD

SEQ ID NO: 517 - protein, 8 aa, synthetic construct
GFTFSSYW

SEQ ID NO: 518 - protein, 8 aa, synthetic construct
GFTFDDYA

SEQ ID NO: 519 - protein, 8 aa, synthetic construct
GFTFSDYY

SEQ ID NO: 520 - protein, 8 aa, synthetic construct
GFTFSSYD

SEQ ID NO: 521 - protein, 8 aa, synthetic construct
GFTFSNAW

SEQ ID NO: 522 - protein, 8 aa, synthetic construct
GFTFSNSD

SEQ ID NO: 523 - protein, 8 aa, synthetic construct
GFTFSNSD

SEQ ID NO: 524 - protein, 8 aa, synthetic construct
GFTFDDYG

SEQ ID NO: 525 - protein, 8 aa, synthetic construct
GFTFSSYS

SEQ ID NO: 526 - protein, 8 aa, synthetic construct
GFTFSSYG

SEQ ID NO: 527 - protein, 10 aa, synthetic construct
IKSKTDGGTT

SEQ ID NO: 528 - protein, 8 aa, synthetic construct
INWNGGST

SEQ ID NO: 529 - protein, 8 aa, synthetic construct
ISGSGGST

SEQ ID NO: 530 - protein, 7 aa, synthetic construct
IGTAGDT

SEQ ID NO: 531 - protein, 8 aa, synthetic construct
VNPNGGST

SEQ ID NO: 532 - protein, 8 aa, synthetic construct
INSDGSST

SEQ ID NO: 533 - protein, 8 aa, synthetic construct
IYSGGSST

SEQ ID NO: 534 - protein, 7 aa, synthetic construct
IYHSGST

SEQ ID NO: 535 - protein, 8 aa, synthetic construct
INPSGGST

SEQ ID NO: 536 - protein, 8 aa, synthetic construct
INPNSGGT

SEQ ID NO: 537 - protein, 6 aa, synthetic construct
ARDGAD

SEQ ID NO: 538 - protein, 6 aa, synthetic construct
AKNGAD

SEQ ID NO: 539 - protein, 6 aa, synthetic construct
AKDGKD

SEQ ID NO: 540 - protein, 6 aa, synthetic construct
AKDGND

SEQ ID NO: 541 - protein, 6 aa, synthetic construct
ARDGND

SEQ ID NO: 542 - protein, 6 aa, synthetic construct
ARDGKD

SEQ ID NO: 543 - protein, 6 aa, synthetic construct
AKNGAD

SEQ ID NO: 544 - protein, 6 aa, synthetic construct
AKDGSD

SEQ ID NO: 545 - protein, 6 aa, synthetic construct
AKDGTD

SEQ ID NO: 546 - protein, 6 aa, synthetic construct
ARDSAD

SEQ ID NO: 547 - protein, 8 aa, synthetic construct
GFTFSSYW

SEQ ID NO: 548 - protein, 8 aa, synthetic construct
GFTFDDYA

SEQ ID NO: 549 - protein, 8 aa, synthetic construct
GFTFSDYY

SEQ ID NO: 550 - protein, 8 aa, synthetic construct
GFTFSSYD

SEQ ID NO: 551 - protein, 8 aa, synthetic construct
GFTFSNAW

SEQ ID NO: 552 - protein, 8 aa, synthetic construct
GFTFSNSD

SEQ ID NO: 553 - protein, 8 aa, synthetic construct
GFTFSNSD

SEQ ID NO: 554 - protein, 8 aa, synthetic construct
GFTFDDYG

SEQ ID NO: 555 - protein, 8 aa, synthetic construct
GFTFSSYS

SEQ ID NO: 556 - protein, 8 aa, synthetic construct
GFTFSSYG

SEQ ID NO: 557 - protein, 10 aa, synthetic construct
IKSKTDGGTT

SEQ ID NO: 558 - protein, 8 aa, synthetic construct
INWNGGST

SEQ ID NO: 559 - protein, 8 aa, synthetic construct
ISGSGGST

SEQ ID NO: 560 - protein, 7 aa, synthetic construct
IGTAGDT

SEQ ID NO: 561 - protein, 8 aa, synthetic construct
VNPNGGST

SEQ ID NO: 562 - protein, 8 aa, synthetic construct
INSDGSST

SEQ ID NO: 563 - protein, 8 aa, synthetic construct
IYSGGSST

SEQ ID NO: 564 - protein, 7 aa, synthetic construct
IYHSGST

SEQ ID NO: 565 - protein, 8 aa, synthetic construct
INPSGGST

SEQ ID NO: 566 - protein, 8 aa, synthetic construct
INPNSGGT

SEQ ID NO: 567 - protein, 6 aa, synthetic construct
ARDGAD

SEQ ID NO: 568 - protein, 6 aa, synthetic construct
AKNGAD

SEQ ID NO: 569 - protein, 6 aa, synthetic construct
AKDGKD

SEQ ID NO: 570 - protein, 6 aa, synthetic construct
AKDGND

SEQ ID NO: 571 - protein, 6 aa, synthetic construct
ARDGND

SEQ ID NO: 572 - protein, 6 aa, synthetic construct
ARDGKD

SEQ ID NO: 573 - protein, 6 aa, synthetic construct
AKNGAD

SEQ ID NO: 574 - protein, 6 aa, synthetic construct
AKDGSD

SEQ ID NO: 575 - protein, 6 aa, synthetic construct
AKDGTD

SEQ ID NO: 576 - protein, 6 aa, synthetic construct
ARDSAD

SEQ ID NO: 577 - protein, 8 aa, synthetic construct
GFTFSSYW

SEQ ID NO: 578 - protein, 8 aa, synthetic construct
GFTFSSYA

SEQ ID NO: 579 - protein, 8 aa, synthetic construct
GFTFSDYY

SEQ ID NO: 580 - protein, 8 aa, synthetic construct
GFTFSSYD

SEQ ID NO: 581 - protein, 8 aa, synthetic construct
GFTFSNAW

SEQ ID NO: 582 - protein, 8 aa, synthetic construct
GFTFSNSD

SEQ ID NO: 583 - protein, 8 aa, synthetic construct
GFTFSNSD

SEQ ID NO: 584 - protein, 8 aa, synthetic construct
GFTFDDYG

SEQ ID NO: 585 - protein, 8 aa, synthetic construct
GFTFSSYS

SEQ ID NO: 586 - protein, 8 aa, synthetic construct
GFTFSSYG

SEQ ID NO: 587 - protein, 10 aa, synthetic construct
IKSKTDGGTT

SEQ ID NO: 588 - protein, 8 aa, synthetic construct
INWNGGST

SEQ ID NO: 589 - protein, 8 aa, synthetic construct
ISGSGGST

SEQ ID NO: 590 - protein, 7 aa, synthetic construct
IGTAGDT

SEQ ID NO: 591 - protein, 8 aa, synthetic construct
VNPNGGST

SEQ ID NO: 592 - protein, 8 aa, synthetic construct
INSDGSST

SEQ ID NO: 593 - protein, 8 aa, synthetic construct
IYSGGSST

SEQ ID NO: 594 - protein, 7 aa, synthetic construct
IYHSGST

SEQ ID NO: 595 - protein, 8 aa, synthetic construct
INPSGGST

SEQ ID NO: 596 - protein, 8 aa, synthetic construct
INPNSGGT

SEQ ID NO: 597 - protein, 6 aa, synthetic construct
ARDGAD

SEQ ID NO: 598 - protein, 6 aa, synthetic construct
AKNGAD

SEQ ID NO: 599 - protein, 6 aa, synthetic construct
AKDGKD

SEQ ID NO: 600 - protein, 6 aa, synthetic construct
AKDGND

SEQ ID NO: 601 - protein, 6 aa, synthetic construct
ARDGND

SEQ ID NO: 602 - protein, 6 aa, synthetic construct
ARDGKD

SEQ ID NO: 603 - protein, 6 aa, synthetic construct
AKNGAD

SEQ ID NO: 604 - protein, 6 aa, synthetic construct
AKDGSD

SEQ ID NO: 605 - protein, 6 aa, synthetic construct
AKDGTD

SEQ ID NO: 606 - protein, 6 aa, synthetic construct
ARDSAD

SEQ ID NO: 607 - protein, 8 aa, synthetic construct
GFTFSSYW

SEQ ID NO: 608 - protein, 8 aa, synthetic construct
GFTFDDYA

SEQ ID NO: 609 - protein, 8 aa, synthetic construct
GFTFSDYY

SEQ ID NO: 610 - protein, 8 aa, synthetic construct
GFTFSSYD

SEQ ID NO: 611 - protein, 8 aa, synthetic construct
GFTFSNAW

SEQ ID NO: 612 - protein, 8 aa, synthetic construct
GFTFSNSD

SEQ ID NO: 613 - protein, 8 aa, synthetic construct
GFTFSNSD

SEQ ID NO: 614 - protein, 8 aa, synthetic construct
GFTFDDYG

SEQ ID NO: 615 - protein, 8 aa, synthetic construct
GFTFSSYS

SEQ ID NO: 616 - protein, 8 aa, synthetic construct
GFTFSSYG

SEQ ID NO: 617 - protein, 8 aa, synthetic construct
IKQDGSEK

SEQ ID NO: 618 - protein, 8 aa, synthetic construct
ISWNSGSI

SEQ ID NO: 619 - protein, 8 aa, synthetic construct
ISSSGSTI

SEQ ID NO: 620 - protein, 7 aa, synthetic construct
IGTAGDT

SEQ ID NO: 621 - protein, 10 aa, synthetic construct
IKSKTDGGTT

SEQ ID NO: 622 - protein, 8 aa, synthetic construct
VSWNGSRT

SEQ ID NO: 623 - protein, 8 aa, synthetic construct
INWNGGST

SEQ ID NO: 624 - protein, 8 aa, synthetic construct
ISSSSSYI

SEQ ID NO: 625 - protein, 8 aa, synthetic construct
ISGSGGST

SEQ ID NO: 626 - protein, 8 aa, synthetic construct
IQCDGSQI

SEQ ID NO: 627 - protein, 11 aa, synthetic construct
VGRSSTSITPV

SEQ ID NO: 628 - protein, 11 aa, synthetic construct
VGRNSTSITPV

SEQ ID NO: 629 - protein, 11 aa, synthetic construct
VGRYSTSITPV

SEQ ID NO: 630 - protein, 11 aa, synthetic construct
VGRQSTSITPV

SEQ ID NO: 631 - protein, 11 aa, synthetic construct
VGRGSLSITPV

SEQ ID NO: 632 - protein, 11 aa, synthetic construct
VGRGSCSITPV

SEQ ID NO: 633 - protein, 11 aa, synthetic construct
VGRGSISITPV

SEQ ID NO: 634 - protein, 11 aa, synthetic construct
VGRGSTSILPV

SEQ ID NO: 635 - protein, 11 aa, synthetic construct
VGRGSTSLTPV

SEQ ID NO: 636 - protein, 11 aa, synthetic construct
VGRGSNSITPV

SEQ ID NO: 637 - protein, 8 aa, synthetic construct
GFTFSSYW

SEQ ID NO: 638 - protein, 8 aa, synthetic construct
GFTFDDYA

SEQ ID NO: 639 - protein, 8 aa, synthetic construct
GFTFSDYY

SEQ ID NO: 640 - protein, 8 aa, synthetic construct
GFTFSSYD

SEQ ID NO: 641 - protein, 8 aa, synthetic construct
GFTFSNAW

SEQ ID NO: 642 - protein, 8 aa, synthetic construct
GFTFSNSD

SEQ ID NO: 643 - protein, 8 aa, synthetic construct
GFTFSNSD

SEQ ID NO: 644 - protein, 8 aa, synthetic construct
GFTFDDYG

SEQ ID NO: 645 - protein, 8 aa, synthetic construct
GFTFSSYS

SEQ ID NO: 646 - protein, 8 aa, synthetic construct
GFTFSSYG

SEQ ID NO: 647 - protein, 8 aa, synthetic construct
IKQDGSEK

SEQ ID NO: 648 - protein, 8 aa, synthetic construct
ISWNSGSI

SEQ ID NO: 649 - protein, 8 aa, synthetic construct
ISSSGSTI

SEQ ID NO: 650 - protein, 7 aa, synthetic construct
IGTAGDT

SEQ ID NO: 651 - protein, 10 aa, synthetic construct
IKSKTDGGTT

SEQ ID NO: 652 - protein, 8 aa, synthetic construct
VSWNGSRT

SEQ ID NO: 653 - protein, 8 aa, synthetic construct
INWNGGST

SEQ ID NO: 654 - protein, 8 aa, synthetic construct
ISSSSSYI

SEQ ID NO: 655 - protein, 8 aa, synthetic construct
ISGSGGST

SEQ ID NO: 656 - protein, 8 aa, synthetic construct
IQCDGSQI

SEQ ID NO: 657 - protein, 11 aa, synthetic construct
VGRSSTSITPV

SEQ ID NO: 658 - protein, 11 aa, synthetic construct
VGRNSTSITPV

SEQ ID NO: 659 - protein, 11 aa, synthetic construct
VGRYSTSITPV

SEQ ID NO: 660 - protein, 11 aa, synthetic construct
VGRQSTSITPV

SEQ ID NO: 661 - protein, 11 aa, synthetic construct
VGRGSLSITPV

SEQ ID NO: 662 - protein, 11 aa, synthetic construct
VGRGSCSITPV

SEQ ID NO: 663 - protein, 11 aa, synthetic construct
VGRGSISITPV

SEQ ID NO: 664 - protein, 11 aa, synthetic construct
VGRGSTSILPV

SEQ ID NO: 665 - protein, 11 aa, synthetic construct
VGRGSTSLTPV

SEQ ID NO: 666 - protein, 11 aa, synthetic construct
VGRGSNSITPV

SEQ ID NO: 667 - protein, 471 aa, synthetic construct
MEFGLSWLFLVAILKGVQCKLAQVTLKESGAEVKKPGESLKISCKASGYSFTNYWIGWVRQMPGKGLEWMGIIYPGDSDTRYSPSFQGQVTISADKSISTAYLQWSSLKASDTAMYYCARQGGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 668 - DNA, 1413 bp, synthetic construct
  source 1..1413: mol_type = other DNA
atggagtttgggctgagctggctttttcttgtcgcgattttaaaaggtgtccagtgcaagcttgcgcaggtcaccttgaaggagtctggggcagaggtgaaaaagccgggggagtctctgaaaatctcctgtaaggcttctggatacagctttaccaattactggatcggctgggtgcgccagatgcccggcaaaggcctggagtggatggggatcatctatcctggtgactctgataccagatacagcccgtccttccaaggccaggtcaccatctcagccgacaagtccatcagcactgcctacctgcagtggagcagcctgaaggcctcggacaccgccatgtattactgtgcgagacaggggggctactactacggtatggacgtctggggccaagggaccacggtcaccgtctcctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 669 - protein, 512 aa, synthetic construct
MDMRVPAQLLGLLLLWFPGSRCAVPVLTQPHSVSESPGKTVTISCTRSSGSIASNYVQWYQQRPGSSPTTVIYEDNQRPSGVPDRFSGSIDMSSNSASLTISGLRTEDEGDYYCQSYDSRNHVVFGGGTQLTVLGGQPKANPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 670 - DNA, 1536 bp, synthetic construct
  source 1..1536: mol_type = other DNA
atggacatgcgcgtgcccgcccagctgctgggcctgctgctgctgtggttccccggctcgcgatgcgcggtgcctgtgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacatgtcgtccaactctgcctccctcaccatctctggactgaggactgaggacgagggtgactattactgtcagtcttatgatagccgcaatcatgtggtattcggcggagggacccagctcaccgttttaggtggtcagcccaaggccaaccccacggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcaggaggtggcgggtctggtggaggcggaagcggtggtggcggatcccaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggaggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactagg

SEQ ID NO: 671 - protein, 467 aa, synthetic construct
MEFGLSWLFLVAILKGVQCKLAEVQLVESGGGVVQPGRSLRLSCAASGFTFSSYAMHWVRQAPGKGLEWVATIYQDGSESEYVDSVKGRFTVSRDDAKNTLYLQMDSLRAEDTATYYCVGRDSTSITPVWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 672 - DNA, 1401 bp, synthetic construct
  source 1..1401: mol_type = other DNA
atggagtttgggctgagctggctttttcttgtcgcgattttaaaaggtgtccagtgcaagcttgcggaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccagagacgacgccaagaacacgctgtatctgcaaatggacagtctgagagccgaggacacagccacgtattactgtgtgggacgagattcgacttcaataacacctgtctggggcctaggaaccctggtcaccgtctcctcagctagcaccaagggcccatcggtcttccccctggcgccctgctccaggagcacctccgagagcacagccgccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaggccgctgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtcctgcgctgtcaaaggcttctaccccagcgacatcgccgtggagtgggagagcaatgggcagccggagaacaactacaagaccacgcctcccgtgctggactccgacggctccttcttcctcgtaagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 673 - protein, 729 aa, synthetic construct
MDMRVPAQLLGLLLLWFPGSRCAEIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGSGATNFSLLKQAGDVEENPGPEFGLSWLFLVAILKGVQCKLAEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 674 - DNA, 2187 bp, synthetic construct
  source 1..2187: mol_type = other DNA
atggacatgcgcgtgcccgcccagctgctgggcctgctgctgctgtggttccccggctcgcgatgcgcggaaattgtgatgacccagagcccggcgaccctgagcgtgagcccgggcgaacgcgcgaccctgagctgccgcgcgagccagagcgtgagcagcaacctggcgtggtatcagcagaaaccgggccaggcgccgcgcctgctgatttatggcgcgagcacccgcgcgaccggcattccggcgcgctttagcggcagcggcagcggcaccgaatttaccctgaccattagcagcctgcagagcgaagattttgcggtgtattattgccagcagtataacaactggccgtggacctttggccagggcaccaaagtggaaattaaaactgtggctgcaccatctgtcttcatcttcccgccatctgattcacagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgtggaagcggagctactaacttcagcctgctgaagcaggctggcgacgtggaggagaaccctggacctgagtttgggctgagctggctttttcttgtcgcgattttaaaaggtgtccagtgcaaactcgcggaagtgcagctggtggaaagcggcggcggcctggtgcagccgggccgcagcctgcgcctgagctgcgcggcgagcggctttacctttgatgattatgcgatgcattgggtgcgccaggcgccgggcaaaggcctggaatgggtgagcggcattagctggaacagcggcagcattggctatgcggatagcgtgaaaggccgctttaccattagccgcgataacgcgaaaaacagcctgtatctgcagatgaacagcctgcgcgcggaagataccgcgctgtattattgcgcgaaagatagccgcggctatggcgattatcgcctgggcggcgcgtattggggccagggcaccctggtgaccgtgagcagcgctagcaccaagggcccatcggtcttccccctggcgccctgctccaggagcacctccgagagcacagccgccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaggccgctgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 675 - protein, 461 aa, synthetic construct
MEFGLSWLFLVAILKGVQCKLAEVQLVESGGGLVQPGGSLRLSCTVSGFTFDDYAVSWVRQAPGQGLEWVSSIGAGGGTYYADSVKGRFTISRDDSKDTLYLQMNSLRAEDTAVYYCAKDGVDWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 676 - DNA, 1383 bp, synthetic construct
  source 1..1383: mol_type = other DNA
atggagtttgggctgagctggctttttcttgtcgcgattttaaaaggtgtccagtgcaagcttgcggaggtgcagctggtggagtctgggggaggcttggtacagcccggagggtccctgagactctcctgtaccgtctctggattcacctttgacgactatgccgtgagctgggtccgccaggctccaggacaagggcttgagtgggtatcatctattggtgctggtggtggcacatactacgcagactctgtgaagggccgattcaccatttccagagacgattccaaggacacgctgtatctgcaaatgaacagcctgagagccgaggacacagccgtgtattactgtgcgaaagacggggtcgactggggccagggaaccctggtcaccgtctcctcagctagcaccaagggcccatcggtcttccccctggcgccctgctccaggagcacctccgagagcacagccgccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgaggccgctgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtcctgcgctgtcaaaggcttctaccccagcgacatcgccgtggagtgggagagcaatgggcagccggagaacaactacaagaccacgcctcccgtgctggactccgacggctccttcttcctcgtaagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 677 - protein, 729 aa, synthetic construct; identical to SEQ ID NO: 673

SEQ ID NO: 678 - DNA, 2187 bp, synthetic construct; identical to SEQ ID NO: 674
  source 1..2187: mol_type = other DNA

SEQ ID NO: 679 - protein, 374 aa, synthetic construct
MTRLTVLALLAGLLASSRAEVQLVESGGGVVQPGRSLRLSCAASGFTFSSYAMHWVRQAPGKGLEWVATIYQDGSESEYVDSVKGRFTVSRDDAKNTLYLQMDSLRAEDTATYYCVGRDSTSITPVWGLGTLVTVSSGGGGSGGGGSGGGGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGKLPATG

SEQ ID NO: 680 - DNA, 1122 bp, synthetic construct
  source 1..1122: mol_type = other DNA
atgacccggctgaccgtgctggccctgctggccggcctgctggcctcctccagggccgaggtgcagctggtggagtccgggggaggcgtggtccagcctgggaggtccctgagactctcctgtgcagcctctggattcaccttcagtagctatgctatgcactgggtccgccaggctccagggaaaggcctggagtgggtggccaccatataccaagatggaagtgagagtgaatatgtggactctgtgaagggccgattcaccgtctccagagacgacgccaagaacacgctgtatctgcaaatggacagtctgagagccgaggacacagccacgtattactgtgtgggacgagattcgacttcaataacacctgtctggggcctaggaaccctggtcaccgtctcctcaggaggtggcgggtctggtggaggcggaagcggtggtggcggatccgcacctgaactcctggggggaccgtcagtcttcctcttccccccaaaacccaaggacaccctcatgatctcccggacccctgaggtcacatgcgtggtggtagacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacacctcgcccccatcccgggatgagctgaccaagaaccaggtcagcctgaggtgccatgtcaaaggcttctatcccagcgacatcgccgtggagtgggagagcaatgggcagccggagaacaactacaagaccacgaagcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaactacctgctactggt